(19) United States
(12) Patent Application Publication
(10) Pub. No.: US 2002/0110808 A1
(43) Pub. Date: Aug. 15, 2002

(54) Toxicant-induced differential gene expression

(76) Inventor: John F. Reidhaar-Olson, Montclair, NJ (US)

Correspondence Address: VICKI G. NORTON, ESQ., BROBECK, PHLEGER AND HARRISON LLP, 12390 EL COMINO REAL, SAN DIEGO, CA, 92130, US

(21) Appl. No.: 09/489,220

(22) Filed: Jan. 21, 2000

(51) Int. Cl. C12Q 1/68; C07H 21/02; C07H 21/04; C12P 19/34

(52) U.S. Cl. 435/6; 435/91.2; 536/23.1

(57) ABSTRACT

The present invention identifies nucleic acids that are differentially expressed in cells exposed to various toxicants, including a common group whose expression is modulated by toxicants that act by differing mechanisms. The nucleic acids so identified and their corresponding protein products have utility as markers for specific and general cytotoxic responses. Utilizing the identified nucleic acids, the invention further provides screening methods to identify and characterize toxicants, screens for identifying antidotes to particular toxiciants and diagnostic methods for detecting toxic responses. The identified nucleic acids and their corresponding gene products also serve as targets for various therapeutics designed to alleviate toxic responses.

### FIELD OF THE INVENTION

This invention relates to the field of toxicology and thus is also related to the fields of cellular biology and pharmacology.

### BACKGROUND OF THE INVENTION

Humans and other living organisms are exposed to a variety of toxicants that alter the biochemical and biophysical homeostasis of the exposed subject. The type of toxicants can vary widely, including, for example, various chemicals, ionizing radiation, metal ions and environmental pollutants. Given the broad array of potential toxicants and their capacity to cause significant harm, it is desirable to develop effective methods for identifying toxicants, investigating the mechanism of their effect and to develop methods and compositions for ameliorating their negative effects.

Two major governmental bodies in the United States have been charged with assessing the toxicity of various commercial products. The Environmental Protection Agency (“EPA”) has been granted the authority to require toxicological testing for new chemicals, but rarely invokes this authority because of cost concerns and because of a desire to minimize delays in commercial products reaching the marketplace. It has been estimated that less than 10% of new chemicals (approximately 2,000 a year) are subjected to a detailed toxicological analysis. More typically, the toxicity of new substances are evaluated relative to similar chemicals for which some toxicological data is known.

In the pharmaceutical arena, the Food and Drug Administration (“FDA”) supervises the toxicity of new pharmaceutical agents. The testing required in seeking New Drug Application is quite stringent and expensive. For example, the tests can extend up to a year or longer in duration and involve a variety of carcinogenicity, mutagenicity and reproduction/fertility tests in multiple species of animals. The requirement for animal testing raises its own set of concerns in view of charges that such testing causes unnecessary animal suffering and that extrapolation of results to humans are of questionable validity. Given these concerns, the use of non-animal assay systems such as cellular based assays in which biochemical markers (i.e., genes) are utilized to assess toxicity is an attractive option to animal studies.

### SUMMARY OF THE INVENTION

The present invention identifies nucleic acids that are differentially expressed in cells exposed to various toxicants, including a common group whose expression is modulated by toxicants that act by differing mechanisms. The nucleic acids so identified and their corresponding protein products have utility as markers for specific and general cytotoxic responses and can be used in a variety of screening methods including, for example, screens to identify toxicants, as well as antidotes to particular toxicants. Such nucleic acids and proteins can also serve as targets for various therapeutics designed to alleviate toxic responses.

Appendix A lists the differentially expressed nucleic acids identified in the present invention. Of these, the expression of a group of nucleic acids is modulated upon exposure to each of several toxicants, indicating that the expression levels of this group of nucleic acids is generally altered in response to a toxic insult. This group is listed in Table 1 and includes:

Putative cyclin G1 interacting protein, EST (W74293), Fatty-acid-coenzyme A ligase (long-chain 3), KIAA0220, KIAA0069, Acinus, Translation initiation factor elF1 (A12/SUI1), Ornithine aminotransferase (gyrate atrophy), Insulin-like growth factor binding protein 1, Metallothionein-1H, F₁F₀-ATPase synthase ƒ subunit, Ring finger protein 5, EST (H73484), XP-C repair complementing protein, Squalene epoxidase, Microsomal glutathione-S-transferase 1, Defender against cell death 1, EST (AA034268), COPII protein, KIAA0917, Corticosteroid binding globulin, Calumenin, Ubiquinol-cytochrome c reductase core protein II, SEC13 (S. cerevisiae)-like 1, EST (R51835), Human chromosome 3p21.1 gene sequence, Glutathione-S-transferase-like, Ribonuclease (RNase A family, 4), Transcription factor Dp-1, MAC30, Cyclin-dependent kinase 4, Multispanning membrane protein, Splicing factor (arginine/serine-rich 1), Cytochrome c-1, Lactate dehydrogenase-A, Pyrroline-5-carboxylate synthetase, Glutamate dehydrogenase, Pyruvate dehydrogenase (lipoamide) beta, Ribosomal protein S6 kinase (90 kD, polypeptide 3), Acetyl-coenzyme A acetyltransferase 2, Proteasome activator subunit 3 (PA28 gamma; Kᵢ), EST (N22016), EST (AI131502), Activating transcription factor 4, Transforming growth factor-beta type III receptor, EST (AA283846), EST (AI310515) and EST (AA805555) (the numbers listed in parentheses being the corresponding GenBank accession number).

One of the differentially expressed nucleic acids has the sequence set forth in SEQ ID NO:1. The invention further includes sequences complementary to the sequence set forth in SEQ ID NO:1, sequences including conservative substitutions, sequences that hybridize to the sequence set forth in SEQ ID NO:1 under stringent conditions and fragments of the foregoing. Thus, the invention includes an isolated nucleic acid comprising a nucleotide sequence selected from the group consisting of: (a) a deoxyribonucleotide sequence complementary to the full-length nucleotide sequence of SEQ ID NO:1; (b) a ribonucleotide sequence complementary to the full-length nucleotide sequence of SEQ ID NO:1; and (c) a nucleotide sequence complementary to the deoxyribonucleotide sequence of (a) or the ribonucleotide sequence of (b). Also provided are isolated nucleic acids that include at least 20 contiguous bases from nucleotides 153 to 224 as set forth in SEQ ID NO:1 or a complementary sequence of the same length.

The nucleic acids identified in the invention can be used to prepare specific probes and primers. Such probes and primers can be used in a variety of screening and diagnostic methods to identify toxicants and toxic conditions. A typical screening method involves determining the expression level of at least two nucleic acids of the invention in a test sample and comparing the expression level in the test sample to the expression level of the same nucleic acids in a control sample. A difference in expression levels for the nucleic acids between the two samples is an indicator of a toxic response in the test sample.

For example, certain screening methods are designed to screen test compounds (e.g., potential therapeutics) for toxicity. Libraries of compounds can be screened by contacting each compound with a cell or population of cells, determining the expression level for one or more of the differentially expressed nucleic acids identified by the invention and comparing the level of expression of these nucleic acids with the expression level of the same nucleic acids in a control cell or population of control cells. A difference in expression levels between the two populations indicates that the compound is a toxicant. Other methods are designed to identify antidotes to known toxicants. Such methods typically involve contacting a test cell or population of test cells with a known toxicant under conditions capable of generating a toxic response; the test cell(s) are further contacted with a test compound that is a potential antidote. If the expression levels for differentially expressed genes in the test cells is similar to the expression levels for a non-toxic state (e.g. in control cells not exposed to a toxicant), such a result indicates that the test compound is an antidote to the toxicant under test.

The invention also provides diagnostic methods for identifying individuals suffering from toxicity. The method is similar to the general screening methods. A sample is obtained from an individual potentially suffering from a toxic condition. Probes and primers that specifically hybridize to the differentially expressed nucleic acids are then utilized in hybridization or amplification procedures to detect whether one or more of the differentially expressed nucleic acids identified by the invention are in fact differentially expressed. A finding that one or more of such nucleic acids is differentially expressed indicates that the individual is reacting to exposure to a toxicant.

In certain screening methods, the expression levels of all or most of the nucleic acids in Table 1 are examined; whereas, in other methods, only a relatively small number of the listed nucleic acids are examined (e.g., 3-10). For instance, the subset of genes can include “stress genes” (e.g., XP-C repair complementing protein, Glutathione-S-transferase, Metallothionein-1H, Heat shock protein 90, cAMP-dependent transcription factor ATF-4 and EST (AI148382). In other instances, the subset of genes can include those that belong to the so-called group of house keeping genes involved in normal cellular activity (e.g., Cytochrome c-1, F₁F₀-ATPase synthase, Ubiquinol-cytochrome c reductase core protein II, Lactate dehydrogenase-A, Pyruvate dehydrogenase E1-beta subunit and NADH dehydrogenase subunit 2). A subset of genes used in other methods includes genes involved in cellular apoptosis (e.g., Acinus and Defender against cell death 1). Certain other screening methods focus on those nucleic acids whose expression is up-regulated or down-regulated relative to controls.

In another aspect, the invention provides systems and methods for conducting reporter assays to identify a toxic response. The reporter assay systems generally include multiple reporter constructs (typically at least 2 or 3), each reporter construct including a different promoter or response element that is from one of the differentially expressed genes of the invention. The promoters or response elements are responsive to a toxicant and are operably linked to a reporter gene such that exposure to toxicant activates the transcription of the reporter gene, thereby generating a detectable signal that is an indicator of a toxic response. The reporter constructs are typically harbored in one or more cells. Normally, the signal detected in test cells is compared with control cells that include the same reporter constructs and are treated identically except for exposure to the test compound.

The invention also provides various kits for conducting toxicity analyses. Certain kits include multiple primer pairs that are effective to prime the amplification of a segment of different differentially expressed nucleic acids of the invention and an enzyme effective at amplifying the segments when supplied with the appropriate nucleotides. Other kits include multiple polynucleotide probes that hybridize under stringent conditions to different differentially expressed nucleic acids of the invention; such kits can also include cells effective for expressing the nucleic acids to which the probes hybridize.

### BRIEF DESCRIPTION OF THE DRAWINGS

FIGS. 1A-1C illustrate dose-response curves showing the effects of three toxicants on BrdU incorporation in HepG2 cells for acetaminophen (IC₅₀≈5 mM), caffeine (IC₅₀≈6 mM), and thioacetamide (IC₅₀=57 mM), respectively. The lines are curve fits of the form y=1/(1+x/IC₅₀).

FIGS. 2A-2C are dose-response curves for expression of clone A108D (activating transcription factor 4; GenBank accession number D90209) and 90-1 (EST AA283846) upon treatment of HepG2 cells for 24 hr with acetaminophen (FIG. 2A), caffeine (FIG. 2B), and thioacetamide (FIG. 2C). Expression was measured by in situ hybridization of ³³P-labelled riboprobes to fixed, permeabilized cells grown and treated in Cytostar-T plates. Relative expression levels are ratios of counts bound in treated wells to counts bound in control wells.

FIGS. 3A-3C show time course/dose-response for expression of selected genes in response to acetaminophen (FIGS. 3A and 3B) and caffeine (FIG. 3C). Expression was measured as described for FIGS. 2A-2C.

FIGS. 4A and 4B are plots of apoptosis measurements in HepG2 cells in response to toxicants. Cells were treated with 20 mM acetaminophen (APAP), 16 mM caffeine (CAF), or 100 mM thioacetamide (THIO). Apoptosis was measured after 6 hr (left-most bar of each pair) and 24 hr (right-most bar of each pair) of treatment, using the annexin V (FIG. 4A) and caspase-3 assays (FIG. 4B).

FIGS. 5A and 5B are comparisons of gene expression changes in HepG2 cells at 2 hr (FIG. 5A) and 18 hr (FIG. 5B) following treatment with 20 mM acetaminophen. Normalized expression values in control and treated samples are plotted. The dashed lines indicate ten-fold up- or down-regulation. The dotted lines indicate the estimated background level.

FIGS. 6A-6C shows the degree of differential gene expression as a function of time in HepG2 cells exposed to 20 mM acetaminophen (FIG. 6A), 16 mM caffeine (FIG. 6B), and 100 mM thioacetamide (FIG. 6C). The rms values are a measure of the degree of expression change without regard to direction, and are defined by ((Σ( Tᵢ-Cᵢ)²)/N)^{½}, where Tᵢ and Cᵢ are the normalized expression values for gene i in treated and control samples, respectively, and N is the total number of genes on the array. Intensities below the background threshold in both control and treated samples were omitted from the calculation.

FIGS. 7A and 7B are comparisons between gene expression data obtained by array hybridization and quantitative RT-PCR. FIG. 7A is a time course of expression of the lactate dehydrogenase-A gene in response to 20 mM acetaminophen, monitored by array (●) or RT-PCR (∘). FIG. 7B is a comparison of array and RT-PCR expression data for genes tested in both assays (see Table 10). In both plots, the logarithms (base 2) of the expression ratios (treated/control) are plotted. Metallothionein gene data (see Table 11) are not included in this plot.

### DETAILED DESCRIPTION

I. Definitions

The term “toxic,” “toxicity,” “cytotoxic,” “cytotoxicity” and other related terms are meant to broadly refer to alterations of the biochemical and biophysical homeostasis of a cell that result in the inhibition of cell growth and/or proliferation and/or cell death and/or alteration of cell function (e.g., down regulation of certain cellular activities) and that cause measurable changes in the expression of one or more genes. Toxicants can act by a number of different mechanisms including, for example, mitochondrial disruption, macromolecular binding, genotoxicity (e.g., DNA modifications), alteration of redox state, and changes in protein concentrations or function. Redox alterations can include, for example, changes in the concentrations of various redox active agents such as superoxides, radicals, peroxides and glutathione levels. Such changes can result in damage to different cellular components (e.g., lipid peroxidation and oxidative damage to DNA). Toxic effects involving DNA include, for example, alterations in nucleic acids and precursors thereto such as DNA strand breaks, DNA strand cross-linking, increases and decreases in superhelicity and oxidative or radiation damage to DNA or nucleotides. Protein alterations associated with cytotoxicity include, but are not limited to, alterations in proteins or amino acids such as denaturation of proteins, misfolding of proteins, formation of covalent adducts between protein and toxicant resulting in alteration of protein activity (e.g., protein unfolding or inhibition of catalytic activity), cross-linking of proteins, formation or breakage of disulfide bonds and other changes associated with oxidation of proteins.

A “toxicant” or “toxic compound” and other related terms is a substance capable of causing a toxic effect, i.e., of altering the biochemical and biophysical homeostasis of a cell, thereby resulting in the inhibition of cell growth and/or proliferation and causing a measureable change in the expression of one or more genes. The term encompasses a diverse group of agents generally including, for example, various chemicals, metals, pollutants and so on. More specifically the terms include, but are not limited to, heavy metals, aromatic hydrocarbons, acids, bases, alkylating agents, peroxides, cross-linking agents, redox active compounds, inflammatory agents, drugs, ethanol, steroids, growth factors. The term also includes non-chemical influences such as UV radiation, heat and X-rays.

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof. A “subsequence” or “segment” refers to a sequence of nucleotides or amino acids that comprise a part of a longer sequence of nucleotides or amino acids (e.g., a polypeptide), respectively.

A “polynucleotide” refers to a single or double-stranded polymer of deoxyribonucleotide or ribonucleotide bases.

The term “target nucleic acid” refers to a nucleic acid (often derived from a biological sample), to which the polynucleotide probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid can refer to the specific subsequence of a larger nucleic acid to which the probe is directed or to the overall sequence (e.g., gene or mRNA) whose expression level it is desired to detect.

A “probe” or “polynucleotide probe” is an nucleic acid capable of binding to a target nucleic acid of complementary sequence through one or more types of chemical bonds, usually through complementary base pairing, usually through hydrogen bond formation, thus forming a duplex structure. The probe binds or hybridizes to a “probe binding site.” A probe can include natural (i.e., A, G, C, or T) or modified bases (7-deazaguanosine, inosine, etc.). A probe can be an oligonucleotide which is a single-stranded DNA. Polynucleotide probes can be synthesized or produced from naturally occurring polynucleotides. In addition, the bases in a probe can be joined by a linkage other than a phosphodiester bond, so long as it does not interfere with hybridization. Thus, probes can include, for example, peptide nucleic acids in which the constituent bases are joined by peptide bonds rather than phosphodiester linkages (see, e.g., Nielsen et al., Science 254, 1497-1500 (1991)). Some probes can have leading and/or trailing sequences of noncomplementarity flanking a region of complementarity.

A “perfectly matched probe” has a sequence perfectly complementary to a particular target sequence. The probe is typically perfectly complementary to a portion (subsequence) of a target sequence. The term “mismatch probe” refer to probes whose sequence is deliberately selected not to be perfectly complementary to a particular target sequence.

A “primer” is a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides, although shorter or longer primers can be used as well. Short primer molecules generally require cooler temperatures to form sufficiently stable hybrid complexes with the template. A primer need not reflect the exact sequence of the template but must be sufficiently complementary to hybridize with a template. The term “primer site” refers to the area of the target DNA to which a primer hybridizes. The term “primer pair” means a set of primers including a 5′ “upstream primer” that hybridizes with the 5′ end of the DNA sequence to be amplified and a 3′ “downstream primer” that hybridizes with the complement of the 3′ end of the sequence to be amplified.

The term “complementary” means that one nucleic acid is identical to, or hybridizes selectively to, another nucleic acid molecule. Selectivity of hybridization exists when hybridization occurs that is more selective than total lack of specificity. Typically, selective hybridization will occur when there is at least about 55% identity over a stretch of at least 14-25 nucleotides, preferably at least 65%, more preferably at least 75%, and most preferably at least 90%. Preferably, one nucleic acid hybridizes specifically to the other nucleic acid. See M. Kanehisa, Nucleic Acids Res. 12:203 (1984).

The terms “polypeptide,” “peptide” and “protein” are used interchangeably to refer to a polymer of amino acid residues. The term also applies to amino acid polymers in which one or more amino acids are chemical analogues of a corresponding naturally occurring amino acids.

The term “operably linked” refers to functional linkage between a nucleic acid expression control sequence (such as a promoter, signal sequence, or array of transcription factor binding sites) and a second polynucleotide, wherein the expression control sequence affects transcription and/or translation of the second polynucleotide.

A “heterologous sequence” or a “heterologous nucleic acid,” as used herein, is one that originates from a source foreign to the particular host cell, or, if from the same source, is modified from its original form. Thus, a heterologous gene in a prokaryotic host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, that has control elements that are capable of effecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding a desired polypeptide) and a promoter. Additional factors necessary or helpful in effecting expression can also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The term “isolated,” “purified” or “substantially pure” means an object species (e.g., a nucleic acid sequence described herein or a polypeptide encoded thereby) is the predominant macromolecular species present (i.e., on a molar basis it is more abundant than any other individual species in the composition), and preferably the object species comprises at least about 50 percent (on a molar basis) of all macromolecular species present. Generally, an isolated, purified or substantially pure composition will comprise more than 80 to 90 percent of all macromolecular species present in a composition. Most preferably, the object species is purified to essential homogeneity (i.e., contaminant species cannot be detected in the composition by conventional detection methods) wherein the composition consists essentially of a single macromolecular species.

The terms “identical” or percent “identity,” in the context of two or more nucleic acids or polypeptides, refer to two or more sequences or subsequences that are the same or have a specified percentage of nucleotides or amino acid residues that are the same, when compared and aligned for maximum correspondence, as measured using a sequence comparison algorithm such as those described below for example, or by visual inspection.

The phrase “substantially identical,” in the context of two nucleic acids or polypeptides, refers to two or more sequences or subsequences that have at least 75%, preferably at least 85%, more preferably at least 90%, 95% or higher nucleotide or amino acid residue identity, when compared and aligned for maximum correspondence, as measured using a sequence comparison algorithm such as those described below for example, or by visual inspection. Preferably, the substantial identity exists over a region of the sequences that is at least about 30 residues in length, preferably over a longer region than 50 residues, more preferably at least about 70 residues, and most preferably the sequences are substantially identical over the full length of the sequences being compared, such as the coding region of a nucleotide for example. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, e.g., Current Protocols in Molecular Biology (Ausubel et al., 1995 supplement).

One useful algorithm for conducting sequence comparisons is PILEUP. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity is the BLAST and the BLAST 2.0 algorithms, which are described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM 62 scoring matrix. The TBLATN program (using protein sequence for nucleotide sequence) uses as defaults a word length (W) of 3, an expectation (E) of 10, and a BLOSUM 62 scoring matrix. (See, e.g., Henikoff & Henikoff, Proc. Natl. Acad. Sci. USA 89:10915 (1989)).

Another indication that two nucleic acid sequences are substantially identical is that the two molecules hybridize to each other under stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide.

A further indication that two nucleic acid sequences or polypeptides are substantially identical is that the polypeptide encoded by the first nucleic acid is immunologically cross reactive with the polypeptide encoded by the second nucleic acid, as described below. The phrases “specifically binds to a protein” or “specifically immunoreactive with,” when referring to an antibody refers to a binding reaction which is determinative of the presence of the protein in the presence of a heterogeneous population of proteins and other biologics. Thus, under designated immunoassay conditions, a specified antibody binds preferentially to a particular protein and does not bind in a significant amount to other proteins present in the sample. Specific binding to a protein under such conditions requires an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

A polypeptide is typically substantially identical to a second polypeptide, for example, where the two peptides differ only by conservative substitutions. A “conservative substitution,” when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well-known in the art. See, e.g., Creighton (1984) Proteins, W. H. Freeman and Company. In addition, individual substitutions, deletions or additions which alter, add or delete a single amino acid or a small percentage of amino acids in an encoded sequence are also “conservatively modified variations.”

The term “naturally occurring” as applied to an object refers to the fact that an object can be found in nature. For example, a polypeptide or polynucleotide sequence that is present in an organism that can be isolated from a source in nature and which has not been intentionally modified by humans in the laboratory is naturally occurring.

The term “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies, more preferably single chain Fv (scFv) antibodies in which a variable heavy and a variable light chain are joined together (directly or through a peptide linker) to form a continuous polypeptide.

A single chain Fv (“scFv” or “scFv”) polypeptide is a covalently linked VH::VL heterodimer which may be expressed from a nucleic acid including VH- and VL-encoding sequences either joined directly or joined by a peptide-encoding linker. Huston, et al. Proc. Nat. Acad. Sci. USA, 85:5879-5883 (1988). A number of structures for converting the naturally aggregated—but chemically separated light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs”. Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface”. This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, Md. (1987).

The term “antigenic determinant” refers to the particular chemical group of a molecule that confers antigenic specificity.

The term “epitope” generally refers to that portion of an antigen that interacts with an antibody. More specifically, the term epitope includes any protein determinant capable of specific binding to an immunoglobulin or T-cell receptor. Specific binding exists when the dissociation constant for antibody binding to an antigen is ≦1 μM, preferably ≦100 nM and most preferably ≦1 nM. Epitopic determinants usually consist of chemically active surface groupings of molecules such as amino acids and typically have specific three dimensional structural characteristics, as well as specific charge characteristics.

The term “specific binding” (and equivalent phrases) refers to the ability of a binding moiety (e.g., a receptor, antibody, ligand or antiligand) to bind preferentially to a particular target molecule (e.g., ligand or antigen) in the presence of a heterogeneous population of proteins and other biologics (i.e., without significant binding to other components present in a test sample). Typically, specific binding between two entities, such as a ligand and a receptor, means a binding affinity of at least about 10⁶ M⁻¹, and preferably at least about 10⁷, 10⁸, 10⁹, or 10¹⁰ M⁻¹.

II. Overview

The present invention provides screening methods, nucleic acids, compositions and kits useful for identifying toxicants and antidotes, as well as diagnosing and treating toxic conditions. The invention is based, in part, on the identification of genes or gene fragments that are differentially expressed in toxic states relative to their expression in non-toxic states (the “differentially expressed” nucleic acids or genes of the invention). Such genes and gene fragments include a set of genes that are differentially expressed in response to a group of toxicants that act via diverse cytotoxic mechanisms. Consequently, these genes can serve as useful general markers of toxic states for a variety of different toxicants.

The invention provides a variety of methods for conducting expression profiling to detect toxic responses. In general, such methods involve determining the expression level of one or more of the differentially expressed nucleic acids identified in the invention in a test sample and comparing the level of expression in the test sample with the level of expression of the same nucleic acid(s) in a control sample. A difference in expression levels between the test and control samples is an indicator of a toxic response. This general approach can be utilized to screen compounds to identify those having toxic characteristics. For example, test cells capable of expressing one or more of the differentially expressed nucleic acids of the invention are contacted with a compound and allowed to generate a toxic response. The level of expression of one or more of the differentially expressed genes of the invention are than assayed using one of a variety of methods for conducting differential gene analysis. If the level of expression is altered relative to a non-toxic state (e.g., a control cell not in contact with a toxicant), then the difference in expression levels indicates that the potential toxicant is in fact a toxin. Such screening methods are useful, for example, in rapidly screening pharmaceutical candidates for toxicity.

The invention also includes related screening techniques to identify antidotes. For example, a test cell capable of expressing a differentially expressed nucleic acid of the invention is exposed to a known toxicant to generate a toxic response. The cell is simultaneously or subsequently contacted with a potential antidote for a sufficient time period to counteract the toxic effect. A reversal in the expression levels of one or more of the differentially expressed nucleic acids of the invention to normal levels or failure of the known toxicant to induce differential expression indicates that the compound being screened is an antidote.

The differentially expressed nucleic acids of the invention can also serve as “fingerprint genes,” namely genes whose expression level or pattern is characteristic of a particular toxic state, exposure to particular toxicant(s) and/or toxic mechanism. Hence, such fingerprint genes can, for example, be utilized to develop primers, probes and custom designed probe arrays for the detection of particular toxic states or the identification of toxicants acting by specific mechanisms, for example. A plurality of fingerprint genes can be utilized to develop expression profiles.

The invention further provides custom arrays and new reporter assays for detecting modulation in the expression of the differentially expressed nucleic acids of the invention. The custom arrays contain probes capable of specifically hybridizing to one or more of the differentially expressed nucleic acids of the invention and can be used for high throughput screening methods such as those just described and as diagnostic tools. The reporter assays utilize cells containing constructs that include a promoter for a differentially expressed gene of the invention in operable linkage to a reporter gene. Activation of the reporter construct in response to a toxic challenge activates transcription of the reporter gene, thereby generating a detectable signal that indicates a toxic response.

Additionally, the invention provides methods for identifying “target genes” and “target gene products.” Certain target genes are responsible for causing toxic effects in cells. These genes and gene products serve as the targets for new pharmaceutical compositions that counteract the toxic effect of these genes and gene products. Thus, screens for compounds capable of interacting with such target genes and gene products can also be utilized to identify antidotes. Other target genes are up-regulated to generate a protective effect in response to a toxic insult. Hence, the invention also includes compositions that increase the synthesis, expression or activity of such genes or gene products, thereby ameliorating toxic effects.

III. Methods for Inducing Differential Gene Expression

Various approaches can be utilized to induce and thus identify differential gene expression resulting from exposure to a toxicant. The genes identified by the following methods are differentially expressed relative to their expression in cells that are not exposed to a toxicant. “Differential expression” as used herein includes quantitative and qualitative differences in the temporal and/or expression patterns of nucleic acids. A gene that is regulated qualitatively can, for example, be activated or inactivated in test cells exposed to toxicant, whereas the activity is opposite for a control cell not exposed to the toxicant. Thus, a qualitatively regulated gene is detectable either in a test or control cell, but not both. In like manner, a qualitatively regulated gene is detectable in either a test or control subject, but not both. Quantitative differences in expression means that expression of a gene is increased or decreased in response to treatment of a cell with a toxicant.

Thus, the expression of the gene is either up-regulated, resulting in increased amounts of transcript, or down-regulated, resulting in decreased amounts of transcript relative to a control not treated with the toxicant. Within this context, the term detectable means that the expression levels have changed sufficiently so that the difference can be determined (preferably quantitatively) according to methods capable of detecting differential expression of genes (e.g., differential display PCR, probe array methods, quantitative PCR, Northern blot analysis and dot blot assays; see infra). In quantitative analyses, the difference in expression between test and control should be a statistically significant difference. A difference is typically considered to be statistically significant if the probability of the observed difference occurring by chance (the p-value) is less than some predetermined level. As used herein a “statistically significant difference” refers to a p-value that is <0.05, preferably <0.01 and most preferably <0.001. Typically, the change or modulation in expression (i.e., up-regulation or down-regulation) is at least about 20%, in still other instances at least 40% or 50%, in yet other instances at least 70% or 80%, and in other instances at least 90% or 100%, although the change can be considerably higher.

A. Toxicants Acting by Specific Mechanisms

Genes that are differentially expressed in response to toxicants that act via a specific mechanism of action can be identified by contacting cultured cells with a single toxicant known to act via a particular cytotoxic mechanism. Toxic compounds are known to act via a variety of different mechanisms including, for example, mitochondrial disruption, alterations in redox state (e.g., lipid peroxidation, and alteration of redox reactive agents such as superoxides, radicals, peroxides and glutathione levels), DNA modifications (e.g., alterations in nucleic acids and precusors thereto such as DNA strand breaks, DNA strand cross-linking, oxidative damage to DNA or nucleotides), protein alterations (e.g., protein denaturation or misfolding, cross-linking of proteins, formation or breakage of disulfide bonds and other changes associated with oxidation of proteins). Hence, one can interrogate which genes are modulated in response to one of these mechanisms by selectively contacting cells with a toxicant that acts by the mechanism of interest. mRNA is subsequently obtained from the contacted cells and the level of expression of the genes determined. Genes that are differentially expressed relative to a non-toxic state (e.g., expression levels in a control sample) indicate which genes are affected by the cytoxic mechanism of the particular toxicant being examined.

In general the methods utilize cells that are responsive to the particular toxicants of interest (i.e., cells whose biochemical and/or biophysical homeostasis is sufficiently altered in response to treatment with the toxicant such that the differential expression of genes can be detected) and which are capable of expressing one or more of the differentially expressed nucleic acids. Typically, a population of cells grown in standard growth media is treated with a solution containing a sufficient concentration of toxicant to cause a significant reduction in cell growth while not decreasing the overall mRNA concentration in the cells. As used herein, a significant reduction in cell growth means that cell proliferation in a cell culture is reduced as a result of contact by the toxicant of interest by at least 10%, in other instances at least 35%, in yet other instances at least 65%, and in still other instances at least 80%. The solution containing the toxicant can include compounds that enhance solubility and the uptake of the toxicant by the cells. Expression of the genes can then be assessed at a single time point or at a variety of different time points to obtain a temporal record of differential expression.

B. Toxicants Acting by Diverse Mechanisms

Separately contacting cultured cells with toxicants known to exert their toxic effects by different mechanisms is a facile approach for identifying a core group of nucleic acids that are differentially expressed in response to a variety of types of toxicants. In general such methods involve contacting different populations of cultured cells with different toxicants, the different toxicants selected to act via differing toxic mechanisms (see previous section). The nucleic acids whose expression is modulated in each population of cells is then determined. The set of differentially expressed genes for each toxicant reflects the different genes affected by a toxicant acting according to a mechanism for that particular toxicant. However, by comparing the differentially expressed nucleic acids for all the cell populations, it is possible to identify a common group of genes that are differentially expressed in response to each of the toxicants. Hence, this group consists of those genes that are differentially expressed in response to a variety of toxic challenges, even toxicants acting via different mechanisms. As set forth in greater detail in Examples 1 and 2 below, in the present invention cultures of HepG2 cells (cells from a human liver cell line) at or near confluency were separately treated with acetaminophen, caffeine and thioacetamide. These toxicants were selected because they are known to exert their toxic effects via diverse mechanisms including mitochondrial disruption, macromolecular binding, genotoxicity, interference with calcium homeostasis and lipid peroxidation (see e.g., Möller and Dargel, Acta pharmacol. et toxicol. 55: 126-132 (1984); Burcham and Harman, Toxicology Letters 50:37-48 (1990); Burcham and Harman, J. Biol. Chem. 266:5049-5054 (1991); D'Ambrosio, Regulatory toxicology and pharmacology 19:243-281 (1994); Casarett and Doull's Toxicology: The Basic Science of Poisons, (Klaasen, C. D., Ed.), McGraw-Hill, New York, (1996)). mRNA was then isolated from the cells at different times and the levels of expression of different genes determined using differential display PCR, probe array methods and various confirmatory methods such as dot blot assays or quantitative RT-PCR (see infra).

Alternatively, a single population of cells can be contacted with multiple toxicants having differing cytotoxic mechanisms to identify a broad range of genes that are differentially expressed in response to a broad range of toxicants. While such an approach simplifies the approach just described and provides broad insight into the identity of genes whose expression is potentially modulated in response to a toxic challenge, it does not allow one to identify the common set of genes that respond to toxicants having different mechanisms of action.

III. Methods for Identifying Toxicant-Induced Gene Expression Changes

Gene expression changes can be monitored by a variety of known methods including, for example, differential display PCR, probe array methods, quantitative reverse transcriptase (RT)-PCR, Northern analysis, and RNase protection, in situ hybridization and reporter assays. Most methods begin with the isolation of RNA (typically mRNA) from a sample and then determination of the level of expression of genes of interest.

A. mRNA Isolation

To measure the transcription level (and thereby the expression level) of a gene or genes, a nucleic acid sample comprising mRNA transcript(s) of the gene(s) or gene fragments, or nucleic acids derived from the mRNA transcript(s) is obtained. A nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA.

In some methods, a nucleic acid sample is the total mRNA isolated from a biological sample; in other instances, the nucleic acid sample is the total RNA from a biological sample. The term “biological sample”, as used herein, refers to a sample obtained from an organism or from components of an organism, such as cells, biological tissues and fluids. In some methods, the sample is from a human patient. Such samples include sputum, blood, blood cells (e.g., white cells), tissue or fine needle biopsy samples, urine, peritoneal fluid, and fleural fluid, or cells therefrom. Biological samples can also include sections of tissues such as frozen sections taken for histological purposes. Often two samples are provided for purposes of comparison. The samples can be, for example, from different cell or tissue types, from different individuals or from the same original sample subjected to two different treatments (e.g., drug-treated and control).

Any RNA isolation technique that does not select against the isolation of mRNA can be utilized for the purification of such RNA samples. For example, methods of isolation and purification of nucleic acids are described in detail in WO 97/10365, WO 97/27317, Chapter 3 of Laboratory Techniques in Biochemistry and Molecular Biology: Hybridization With Nucleic Acid Probes, Part I. Theory and Nucleic Acid Preparation, (P. Tijssen, ed.) Elsevier, N.Y. (1993); Chapter 3 of Laboratory Techniques in Biochemistry and Molecular Biology: Hybridization With Nucleic Acid Probes, Part 1. Theory and Nucleic Acid Preparation, (P. Tijssen, ed.) Elsevier, N.Y. (1993); and Sambrook et al., Molecular Cloning: A Laboratory Manual, Cold Spring Harbor Press, N.Y., (1989); Current Protocols in Molecular Biology, (Ausubel, F. M. et al., eds.) John Wiley & Sons, Inc., New York (1987-1993). Large numbers of tissue samples can be readily processed using techniques known in the art, including, for example, the single-step RNA isolation process of Chomczynski, P. described in U.S. Pat. No. 4,843,155.

B. Differential Display PCR

Differential display PCR (DD PCR) is one method that is useful for identifying genes that have been differentially expressed under different sets of conditions. DD PCR utilizes a modification of the well-established PCR technique (see, e.g., U.S. Pat. Nos. 4,683,202 and 4,683,195) in which a primer pair consisting of a primer that hybridizes to the poly A tail of the mRNA and an arbitrary primer is used to amplify various segments of the mRNAs contained within a sample. The resulting amplification products are separated on a sequencing gel. Comparison of bands on separate gels obtained for test and control samples allows for the identification of differentially expressed genes. Bands that are differentially expressed can be excised and analyzed further to determine the identity of the differentially expressed gene.

More specifically, the method begins by reverse transcribing isolated RNA into a single-stranded cDNA according to known methods. The resulting cDNA is then amplified using a reverse primer (the “anchor primer”) that contains an oligo dT stretch of nucleotides at its 5′ end (generally about eleven nucleotides long) that hybridizes with the poly (A) tail of the mRNA or to the complement of the cDNA reverse transcribed from an mRNA poly(a) tail. The primer also typically includes one or two additional nucleotides at its 3′ end to increase the specificity of the reverse primer and anchor the primer to a particular segment that includes the poly (A) segment. Because only a subset of the mRNA derived sequences hybridize to such primers, the additional nucleotides allow the primers to amplify only a subset of the mRNA derived sequences present in the sample. The forward primer is typically a primer of arbitrary sequence and generally ranges from about 9 to 13 nucleotides in length, more typically about 10 nucleotides in length.

By using arbitrary primer sequences, the resulting amplified nucleic acids are of variable length and can be separated on a standard denaturing sequencing gel. The pattern of amplified products from two or more cells can be displayed on sequencing gels and compared. Differences in the banding patterns between the gels indicate genes that potentially are differentially expressed. Once such sequences have been so identified, further analyses should be undertaken using alternate techniques such as those described below to corroborate the DD PCR results. As described more fully in Example 1, differential display results in the present invention were confirmed using dot blot assays.

DD-PCR has an advantage relative to certain other methods of differential gene expression detection in that no prior knowledge of gene sequences is required. Further, because the PCR conditions are conducted under relatively low stringency conditions such that only 5-6 bases at the 3′ end of each primer need match a potential template, with a sufficient number of primers it is possible to detect most expressed genes.

Further guidance regarding the use of DD PCR can be found in a number of sources including, for example, U.S. Pat. Nos. 5,262,311; 5,599,672; and Liang, P. and Pardee, A. B., Science 257:967-971 (1992); Liang, P., et al., Methods of Enzymol. 254:304-321 (1995); Liang, P. et al, Nucl. Acids Res. 22:5763-5764 (1994); Liang, P. and Pardee, A. B., Curr. Opin. in Immunology 7:274-280 (1995); and Reeves, S. A., et al., BioTechniques 18:18-20 (1995), each of which is incorporated by reference in its entirety.

C. Probe Arrays

Array-based expression monitoring is another useful approach for detecting differential gene expression and was utilized in the present invention to identify many of the differentially expressed genes of the invention (see Example 2). This approach can be used to achieve high throughput analysis. The arrays utilized in differential gene expression analysis can be of a variety of differing types, depending in part upon whether the gene and/or gene fragments to be detected are known in advance of an experiment. For example, some arrays contain short polynucleotide probes, while other arrays contain full-length cDNAs. Regardless of the nature of the probe, the probes are typically attached to some type of support.

In probe array methods, once nucleic acids have been obtained from a test sample, they typically are reversed transcribed into labeled cDNA, although labeled mRNA can be used directly. The test sample containing the labeled nucleic acids is then contacted with the probes of the array. After allowing a period for targets to hybridize to the probes, the array is typically subjected to one or more high stringency washes to remove unbound target and to minimize nonspecific binding to the nucleic acid probes of the arrays. Binding of target nucleic acid, and thus detection of expressed genes in the sample, is detected using any of a variety of commercially available scanners and accompanying software programs.

General methods for using expression arrays are described in WO 97/10365, PCT/US/96/143839 and WO 97/27317, each of which are incorporated by reference in their entirety. Additional discussion regarding the use of microarrays in expression analysis can be found, for example, in Duggan, et al., Nature Genetics Supplement 21:10-14 (1999); Bowtell, Nature Genetics Supplement 21:25-32 (1999); Brown and Botstein, Nature Genetics Supplement 21:33-37 (1999); Cole et al., Nature Genetics Supplement 21:38-41 (1999); Debouck and Goodfellow, Nature Genetics Supplement 21:48-50 (1999); Bassett, Jr., et al., Nature Genetics Supplement 21:51-55 (1999); and Chakravarti, Nature Genetics Supplement 21:56-60 (1999), each of which is incorporated herein by reference in its entirety.

1. Types of Arrays

The probes utilized in the arrays of the present invention can include, for example, synthesized probes of relatively short length (e.g., a 20-mer or a 25-mer), cDNA (full length or fragments of gene), amplified DNA, fragments of DNA (generated by restriction enzymes, for example) and reverse transcribed DNA. For a review on different types of microarrays, see for example, Southern et al., Nature Genetics Supplement 21:5-9 (1999), which is incorporated herein by reference.

Synthesized Arrays:

The type of arrays utilized in expression analysis and which can be prepared for use in the foregoing methods fall into two general categories: custom arrays and generic arrays. Custom arrays are useful for detecting the presence and/or concentration of particular mRNA sequences that are known in advance. In such arrays, nucleic acid probes can be selected to hybridize to particular preselected subsequences of mRNA gene sequences or amplification products prepared from them. In some instances, such arrays can include a plurality of probes for each mRNA or amplification product to be detected. The differentially expressed nucleic acids of the invention can be utilized in preparing custom arrays specific for a particular toxic state or for a common set of genes whose expression is modulated by a variety of different toxicants (see infra).

The second type of array is sometimes referred to as a generic array because the array can be used to analyze mRNAs or amplification products generated therefrom irrespective of whether the sequence is known in advance of the analysis. Generic arrays can be further subdivided into additional categories such as random, haphazardly selected, or arbitrary probe sets. In other instances, a generic array can include all the possible nucleic acid probes of a particular pre-selected length.

A random nucleic acid array is one in which the pool of nucleotide sequences of a particular length does not significantly vary from a pool of nucleotide sequences selected in a blind or unbiased manner form a collection of all possible sequences of that length. Arbitrary or haphazard nucleotide arrays of nucleic acid probes are arrays in which the probe selection is made without identifying and/or preselecting target nucleic acids. Although arbitrary or haphazard nucleotide arrays can approximate or even be random, the methods by which the array are generated do not assure that the probes in the array in fact satisfy the statistical definition of randomness. The arrays can reflect some nucleotide selection based on probe composition, and/or non-redundancy of probes, and/or coding sequence bias; however, such probe sets are still not chosen to be specific for any particular genes.

Alternatively, generic arrays can include all possible nucleotides of a given length; that is, polynucleotides having sequences corresponding to every permutation of a sequence. When a probe contains up to 4 bases (A, G, C, T) or (A, G, C, U) or derivatives of these bases, an array having all possible nucleotides of length X contains substantially 4^{X} different nucleic acids (e.g., 16 different nucleic acids for a 2 mer, 64 different nucleic acids for a 3 mer, 65536 different nucleic acids for an 8 mer). Some small number of sequences can be absent from a pool of all possible nucleotides of a particular length due to synthesis problems, and inadvertent cleavage.

In some applications, it is advantageous to utilize polynucleotide arrays containing collections of pairs of nucleic acid probes for each of the RNAs being monitored. In such instances, each probe pair includes a probe (e.g., a 20-mer or a 25 -mer) that is perfectly complementary to a subsequence of a particular mRNA or amplification product generated therefrom, and a companion probe that is identical except for a single base difference in a central position. The mismatch probe of each pair can serve as a internal control for hybridization specificity. See for example, Lockhart, et al., Nature Biotechnology 14:1675-1680 (1996); and Lipschutz, et al., Nature Genetics Supplement 21: 20-24, 1999, which are incorporated by reference herein in their entirety.

cDNA Arrays:

Instead of using arrays containing synthesized probes, the probes can instead be full length cDNA molecules or fragments thereof which are attached to a solid support. Expression analyzes conducted using such probes are described, for example, by Schena et al. (Science 270:467-470 (1995); and DeRisi et al. (Nature Genetics 14:457-460 (1996)), which are incorporated herein by reference in their entirety.

2. Methods of Detection

After hybridization of control and target samples to an array containing one or more probe sets as described above and optional washing to remove unbound and nonspecifically bound probe, the hybridization intensity for the respective samples is determined for each probe in the array. For fluorescent labels, hybridization intensity can be determined by, for example, a scanning confocal microscope in photon counting mode. Appropriate scanning devices are described by e.g., U.S. Pat. No. 5,578,832 to Trulson et al., and U.S. Pat. No. 5,631,734 to Stern et al. (both of which are incorporated by reference in their entirety) and are available from Affymetrix, Inc., under the GeneChip™ label. Some types of label provide a signal that can be amplified by enzymatic methods (see Broude, et al., Proc. Natl. Acad. Sci. U.S.A. 91, 3072-3076 (1994)). A variety of other labels are also suitable including, for example, radioisotopes, chromophores, magnetic particles and electron dense particles.

Optionally, the hybridization signal of matched probes can be compared with that of corresponding mismatched or other control probes. Binding of mismatched probe serves as a measure of background and can be subtracted from binding of matched probes. A significant difference in binding between a perfectly matched probe and a mismatched probe signifies that the nucleic acid to which the matched probes are complementary is present. Binding to the perfectly matched probes is typically at least 1.2, 1.5, 2, 5 or 10 or 20 times higher than binding to the mismatched probes.

In a variation of the above method, nucleic acids are not labeled but are detected by template-directed extension of a probe hybridized to a nucleic acid strand with the nucleic acid strand serving as a template. The probe is extended with a labeled nucleotide, and the position of the label indicates, which probes in the array have been extended. By performing multiple rounds of extension using different bases bearing different labels, it is possible to determine the identity of additional bases in the tag than are determined through complementarity with the probe to which the tag is hybridized. The use of target-dependent extension of probes is described by U.S. Pat. No. 5,547,839, which is incorporated by reference in its entirety.

3. Analysis of Hybridization Patterns

The position of label is detected for each probe in the array using a reader, such as described by U.S. Pat. No. 5,143,854, WO 90/15070, and Trulson et al., U.S. Pat. No. 5,578,832, each of which is incorporated by reference in its entirety. For customized arrays, the hybridization pattern can then be analyzed to determine the presence and/or relative amounts or absolute amounts of known mRNA species in samples being analyzed as described in e.g., WO 97/10365. Comparison of the expression patterns of two samples is useful for identifying mRNAs and their corresponding genes that are differentially expressed between the two samples.

The quantitative monitoring of expression levels for large numbers of genes can prove valuable in elucidating gene function, exploring the mechanism(s) associated with a toxicant, and for the discovery of potential therapeutic and diagnostic targets and methods.

D. Quantitative RT-PCR

A variety of so-called “real time amplification” methods or “real time quantitative PCR” methods can also be utilized to determine the quantity of mRNA present in a sample by measuring the amount of amplification product formed during an amplification process. Fluorogenic nuclease assays are one specific example of a real time quantitation method which can be used successfully with the methods of the present invention (see Example 2). The basis for this method of monitoring the formation of amplification product is to measure continuously PCR product accumulation using a dual-labeled fluorogenic oligonucleotide probe—an approach frequently referred to in the literature simply as the “TaqMan” method.

The probe used in such assays is typically a short (ca. 20-25 bases) polynucleotide that is labeled with two different fluorescent dyes. The 5′ terminus of the probe is typically attached to a reporter dye and the 3′ terminus is attached to a quenching dye, although the dyes could be attached at other locations on the probe as well. The probe is designed to have at least substantial sequence complementarity with the probe binding site. Upstream and downstream PCR primers that bind to flanking regions of the locus are also added to the reaction mixture.

When the probe is intact, energy transfer between the two fluorophors occurs and the quencher quenches emission from the reporter. During the extension phase of PCR, the probe is cleaved by the 5′ nuclease activity of a nucleic acid polymerase such as Taq polymerase, thereby releasing the reporter from the polynucleotide-quencher and resulting in an increase of reporter emission intensity which can be measured by an appropriate detector.

One detector which is specifically adapted for measuring fluorescence emissions such as those created during a fluorogenic assay is the ABI 7700 manufactured by Applied Biosystems, Inc. in Foster City, Calif. Computer software provided with the instrument is capable of recording the fluorescence intensity of reporter and quencher over the course of the amplification. These recorded values can then be used to calculate the increase in normalized reporter emission intensity on a continuous basis and ultimately quantify the amount of the mRNA being amplified.

Additional details regarding the theory and operation of fluorogenic methods for making real time determinations of the concentration of amplification products are described, for example, in U.S. Pat No. 5,210,015 to Gelfand, U.S. Pat. No. 5,538,848 to Livak, et al., and U.S. Pat. No. 5,863,736 to Haaland, as well as Heid, C. A., et al., Genome Research, 6:986-994 (1996); Gibson, U. E. M, et al., Genome Research 6:995-1001 (1996); Holland, P. M., et al., Proc. Natl. Acad. Sci. USA 88:7276-7280, (1991); and Livak, K. J., et al., PCR Methods and Applications 357-362 (1995), each of which is incorporated by reference in its entirety.

E. Dot Blot Assays

Another option for detecting differential gene expression includes spotting a solution containing a nucleic acid known to be differentially expressed on a support. Spotting can be performed robotically to increase reproducibility using an instrument such as the BIODOT instrument manufactured by Cartesian Technologies, Inc., for example. The nucleic acids are typically attached to the support using UV cross-linking methods that are known in the art. Labeled cDNA clones prepared from a mRNA sample of interest are treated to remove self-annealing or annealing between different clones and then contacted with the nucleic acids bound to the support and allowed sufficient time to hybridize with the nucleic acids on the support. Supports are washed to remove unhybridized clones. The formation of hybridized complexes can be detected using various known techniques including, for example, exposing a phosphor screen and subsequent scanning using a phosphorimager (e.g., such as available from Molecular Dynamics). This method can be repeated with mRNA obtained from test cells treated with toxicant and control cells not treated with toxicant to identify genes that are differentially expressed. As described further in Example 1, such methods were utilized in the present invention to confirm the results obtained by DD PCR. For further guidance on such methods, see, e.g., Sambrook, et al., Molecular Cloning: A Laboratory Manual, 2nd ed., Cold Spring Harbor Laboratory Press (1989).

F. In situ Hybridization

This approach involves the in situ hybridization of labeled probes to one or more of the differentially expressed genes of interest. Because the method is performed in situ, it has the advantage that it is not necessary to prepare RNA from the cells. The method involves initially fixing test cells to a support (e.g., the walls of a microtiter well) and then permeabilizing the cells with an appropriate permeabilizing solution. A solution containing the labeled probes is then contacted with the cells and the probes allowed to hybridize with the complementary differentially expressed genes. Excess probe is digested, washed away and the amount of hybridized probe measured. This approach is described in greater detail in Example 1 below; see also Harris, D. W., Anal. Biochem. 243:249-256 (1996); Singer, et al., Biotechniques 4:230-250 (1986); Haase et al., Methods in Virology, vol. VII, pp. 189-226 (1984); and Nucleic Acid Hybridization: A Practical Approach (Hames, et al., Eds.), (1987), each of which is incorporated by reference in its entirety.

G. Reporter Assays

Differential gene expression can also be detected utilizing reporter assays. These assays utilize cells harboring a reporter construct that includes a promoter for a differentially expressed nucleic acid that is operably linked to a reporter gene. Activation of the promoter in response to exposure of the cell to an appropriate toxicant results in the expression of the reporter gene that yields a detectable product. Such assays based upon the differentially expressed nucleic acids of the present invention are described further below. Certain types of reporter assays are discussed in U.S. Pat. No. 5,811,231 to Farr, et al., which is incorporated by reference in its entirety.

H. Subtractive Hybridization

This approach typically includes isolating mRNA from two different sources (e.g., a test cell treated with toxicant and a control cell not treated with toxicant). The isolated mRNA from one of the sources is typically reverse-transcribed to form a labeled cDNA. The resulting single-stranded is hybridized to a large excess of mRNA from the second closely related cell. After hybridization, the cDNA:mRNA hybrids are removed using standard techniques. The remaining “subtracted” labeled cDNA can then be used to screen a cDNA or genomic library of the same cell population to identify those genes that are potentially differentially expressed. See, for example, Sargent, T. D., Meth. Enzymol. 152:423-432 (1987); and Lee et al., Proc. Natl. Acad. Sci. USA, 88:2825-2830 (1991).

I. Differential Screening

This technique involves the duplicate screening of a cDNA library in which one copy of the library is screened with a total cell cDNA probe corresponding to the mRNA population of one cell type. The duplicate copy of the cDNA library is screened with a total cDNA probe corresponding to the mRNA population of the second cell type. For instance, one cDNA probe corresponds to the total cell cDNA probe of a cell obtained from a control subject not exposed to a toxicant. Whereas, the second cDNA probe corresponds to the total cell cDNA probe of the same cell type obtained from a subject exposed to the toxicant of interest. Clones that hybridize to one probe but not the other potentially represent clones derived from differentially expressed genes. Such methods are described, for example, by Tedder, T. F., et al., Proc. Natl. Acad. Sci. USA 85:208-212 (1988).

IV. Differentially Expressed Nucleic Acids and Expression Profiles

A. General

The present invention has utilized DD PCR, probe array methods and various confirmatory methods to identify 474 genes or gene fragments (i.e., Expressed Sequence Tags (ESTs)) whose expression is modulated in response to the toxicants acetaminophen, caffeine or thioacetamide, i.e., the “differentially expressed nucleic acids” (or genes or gene fragments) of the invention (see Appendix A). The genes identified include known genes, but these genes are nonetheless important as markers of toxicity. The invention also includes a novel EST (SEQ ID NO:1), that can be used as a toxicity marker. Some of the identified genes or gene fragments are differentially expressed in response to only one or two of the toxicants. However, a group of 48 genes or ESTs are differently expressed in response to all three toxicants. The fact that this group of genes are differently expressed with three toxicants that act via distinct mechanisms indicates that these genes or gene fragments are important general markers of a toxic response generated by cells. The genes or gene fragments so modulated are listed in Table 1. Unless otherwise stated, the accession numbers used to identify the differentially expressed nucleic acids are GenBank accession numbers.

The differentially expressed nucleic acids of the invention include “fingerprint genes” and “target genes.” Fingerprint genes include nucleic acids whose expression level correlates with a particular toxic state, mechanism or toxicant(s). For example, different fingerprint genes can be differentially expressed for different toxicants or groups of toxicants. Particular fingerprint genes that correlate with specific mechanisms can also be identified. Alternatively, as with the present invention, the fingerprint genes can comprise a group of genes that are differentially expressed by toxicants acting by diverse mechanisms (see Table 1). As described more fully below, fingerprint genes can be utilized in the development of a variety of different screening and diagnostic methods to identify toxicants or toxic states.

TABLE 1
Common group of nucleic acids differentially expressed from
exposure to acetaminophen, caffeine and thioacetamide

| GenBank | ¹ |
|---|---|
| Accession Number | Name |
| H93328 | Putative cyclin G1 interacting protein |
| W74293 | EST, highly similar to laminin B1 |
| W31074 | Fatty-acid -coenzyme A ligase, long-chain 3 |
| R84893 | KIAA0220 |
| H20652 | KIAA0069 |
| H75861 | Acinus |
| R51607 | Translation initiation factor eIF1(A12/SUI1) |
| AA446819 | Ornithine aminotransferase (gyrate atrophy) |
| AA233079 | Insulin-like growth factor binding protein 1 |
| H77766 | Metallothionein 1H |
| N22016 | EST for clone A124-6 |
| AI131502 | EST, similar to ubiquitin hydrolase |
| D90209 | Activating transcription factor 4 |
| H38623 | F₁F₀-ATPase synthase ƒ subunit |
| AA402960 | Ring finger protein 5 |
| H73484 | EST |
| AA489678 | XP-C repair complementing protein |
| RO1118 | Squalene epoxidase |
| AA495936 | Microsomal glutathione-S-transferase 1 |
| AA455281 | Defender against cell death 1 |
| AA034268 | EST |
| AA406332 | COPII protein, SEC23p homolog |
| AA028034 | KIAA0917 (vesicle transport-related protein) |
| H90815 | Corticosteroid binding globulin |
| R78585 | Calumenin |
| R12802 | Ubiquinol-cytochrome c reductase core protein II |
| AA496784 | SEC13 (S. cerevisiae)-like 1 |
| R51835 | EST |
| H94897 | Human chromosome 3p21.1 gene sequence |
| AA441895 | Glutathione-S-transferase-like |
| T60223 | Ribonuclease, RNase A family, 4 |
| W33012 | Transcription factor Dp-1 |
| N79230 | MAC30 |
| AA486312 | Cyclin-dependent kinase 4 |
| AA127685 | Multispanning membrane protein |
| T65902 | Splicing factor, arginine/serine-rich 1 |
| AA447774 | Cytochrome c-1 |
| H05914 | Lactate dehydrogenase-A |
| AA143509 | Pyrroline-5-carboxylate synthetase |
| R54424 | Glutamate dehydrogenase |
| AA521401 | Pyruvate dehydrogenase (lipoamide) beta |
| H55921 | Ribosomal protein S6 kinase, 90 kD, polypeptide 3 |
| R25823 | Acetyl-coenzyme A acetyltransferase 2 |
| AA486324 | Proteasome activator subunit 3 (PA28 gamma; K₁) |
| L07594 | Transforming growth factor-beta type III receptor |
| AA283846 | EST |
| AI310515 | EST |

¹Nucleic acids listed above dividing line were up-regulated, those below the line were down-regulated.

Expression levels for combinations of differentially expressed genes, in particular fingerprint genes, can be used to develop “expression profiles” that are characteristic of a particular toxic state associated with a particular toxicant (or group of toxicants) or a particular toxic mechanism (or group of mechanisms). Expression profiles as used herein refers to the pattern of gene expression corresponding to at least two differentially expressed genes. Typically, an expression profile includes at least 3, 4 or 5 differentially expressed genes, but in other instances can include at least 7, 8, 9, 10, 12, 14, 16, 18, 20, 25, 30, 35, 40, 45, 50 or more differentially expressed genes; in some instances, expression profiles include all of the differentially expressed genes known for a particular state or associated with one or more toxicants.

In some instances, expression profiles are generated for the genes differentially expressed in response to a particular toxicant or one or more toxicants acting via a particular cytotoxic mechanism (i.e., fingerprint genes). Alternatively, expression profiles can include differentially expressed genes selected from a group such as those listed in Table 1 that are differentially expressed in response to toxicants that have differing mechanisms of action.

The pattern of expression associated with gene expression profiles can be defined in several ways. For example, a gene expression profile can be the relative transcript level of any number of particular differentially expressed genes. In other instances, a gene expression profile can be defined by comparing the level of expression of a variety of genes in one state to the level of expression of the same genes in another state (e.g., test cell exposed to a toxicant and a control cell not exposed). For example, genes can be up-regulated, down-regulated, or remain at substantially the same level in both states.

A target gene is a nucleic acid that affects cytotoxicity. Hence, a target gene and its corresponding product can be a causative agent of toxicity or a gene expressed to ameliorate toxicity. In the latter instance, up-regulation of the target gene product has a protective function. Given their role in toxicity, target genes are useful targets for the development of compound discovery programs and pharmaceutical development such as described infra. In some instances, a fingerprint gene can be a target gene and vice versa.

The differentially expressed nucleic acids of the invention generally include naturally occurring, synthetic and intentionally manipulated sequences (e.g., nucleic acids subjected to site-directed mutagenesis). The differentially expressed nucleic acids of the invention also include sequences that are complementary to the listed sequences, as well as degenerate sequences resulting from the degeneracy of the genetic code. Thus, the differentially expressed nucleic acids include: (a) nucleic acids having sequences corresponding to the sequences as provided in the listed GenBank accession number; (b) nucleic acids that encode amino acids encoded by the nucleic acids of (a); (c) a nucleic acid that hybridizes under stringent conditions to a complement of the nucleic acid of (a); and (d) nucleic acids that hybridize under stringent conditions to, and therefor are complements of, the nucleic acids described in (a) through (c). The differentially expressed nucleic acids of the invention also include: (a) a deoxyribonucleotide sequence complementary to the full-length nucleotide sequences corresponding to the listed GenBank accession numbers; (b) a ribonucleotide sequence complementary to the full-length sequence corresponding to the listed GenBank accession numbers; and (c) a nucleotide sequence complementary to the deoxyribonucleotide sequence of (a) and the ribonucleotide sequence of (b). The differentially expressed nucleic acids of the invention further include fragments thereof. For example, nucleic acids including 10, 12, 14, 16, 18, 20, 22, 24, 26, 28, 30, 40, 50, 60, 70, 80, 90, 100, 125, 150, 175, 200, 225, 250, 275 or 300 contiguous nucleotides (or any number of nucleotides therebetween) from a differentially expressed nucleic acid are included. Such fragments are useful, for example, as primers and probes for the differentially expressed nucleic acids of the invention.

In some instances, the differentially expressed nucleic acids include conservatively modified variations. Thus, for example, in some instances, the nucleic acids of the invention are modified. One of skill will recognize many ways of generating alterations in a given nucleic acid construct. Such well-known methods include site-directed mutagenesis, PCR amplification using degenerate polynucleotides, exposure of cells containing the nucleic acid to mutagenic agents or radiation and chemical synthesis of a desired polynucleotide (e.g., in conjunction with ligation and/or cloning to generate large nucleic acids). See, e.g., Giliman and Smith (1979) Gene 8:81-97, Roberts et al. (1987) Nature 328: 731-734). When the differentially expressed nucleic acids of the invention are incorporated into vectors, the nucleic acids can be combined with other sequences including, but not limited to, promoters, polyadenylation signals, restriction enzyme sites and multiple cloning sites. Thus, the overall length of the nucleic acid can vary considerably.

Certain differentially expressed nucleic acids of the invention include polynucleotides that are substantially identical to a polynucleotide sequence as set forth in SEQ ID NO:1. Such nucleic acids can function as new markers for cytotoxicity. For example, the invention includes polynucleotide sequences that are at least 90%, 92%, 94% or 96% identical to the polynucleotide sequence as set forth in SEQ ID NO: 1 over a region of at least 250 nucleotides in length. In other instances, the region of similarity exceeds 250 nucleotides in length and extends for at least 300, 350, 400, 450 or 500 nucleotides in length, or over the entire length of the sequence.

Other differentially expressed nucleic acids of the invention include polynucleotides that are substantially identical to a polynucleotide sequence corresponding to bases 153 to 224 of SEQ ID NO:1. These nucleic acids include polynucleotides that are typically at least 75% identical to the polynucleotide sequence of bases 153 to 224 of SEQ ID NO:1 over a region of at least 30 nucleotides in length. In other instances, the such polynucleotides are at least 80% or 85% identical, in still other instances at least 90% or 95% identical to a polynucleotide sequence corresponding to nucleotides 153 to 224 of SEQ ID NO:1. The region of similarity can extend beyond 30 nucleotides to include, for example, 40, 45, 50, 55, 60 or 65 nucleotides, or the entire sequence.

As described above, sequence identity comparisons can be conducted using a nucleotide sequence comparison algorithm such as those know to those of skill in the art. For example, one can use the BLASTN algorithm. Suitable parameters for use in BLASTN are wordlength (W) of 11, M=5 and N=−4 and the identity values and region sizes just described.

B. Preparation of Differentially Expressed Genes

Although some of the differentially expressed nucleic acids of the invention are fragments of genes, these ESTs can be utilized to identify the corresponding full-length gene utilizing a variety of known techniques. For example, the entire coding sequence can be obtained from an EST using the RACE method (see, e.g., Chenchik, et al., Clonetechniques (X) 1:5-8 (1995); Bames, Proc. Nat. Acad. Sci. USA 91:2216-2220 (1994); and Cheng, et al., Proc. Natl. Acad. Sci. USA 91:5695-5699 (1994)). PCR technology can also be utilized to isolate a full-length cDNA sequence. For example, RNA can be isolated according to the methods described above from an appropriate source. A reverse transcription reaction can be performed on the RNA using a polynucleotide primer specific for the most 5′ end of the amplified fragment for the priming of first strand synthesis. The resulting RNA/DNA hybrid can then be “tailed” with guanines using a standard terminal transferase reaction, the hybrid can then be digested with RNAase H, and second strand synthesis can then be primed with a poly-C primer. Thus, cDNA sequences upstream of the amplified fragment can easily be isolated (see, e.g., Sambrook, et al., Molecular Cloning: A Laboratory Manual, 2nd ed., Cold Spring Harbor Laboratory Press (1989)).

In still another approach, the identified markers can be used to identify and isolate cDNA sequences. The EST sequences provided by the invention can be used as hybridization probes to screen cDNA libraries using standard techniques. Comparison of the cloned cDNA sequence with known sequences can be performed using a variety of computer programs and databases, such as those listed above in the sections describing sequence identity. ESTs can be used as hybridization probes to screen genomic libraries. Once partial genomic clones are identified, full-length genes can be isolated using chromosomal walking (also sometimes referred to as “overlap hybridization”). See, e.g, Chinault and Carbon, Gene 5:111-126, (1979).

The differentially expressed nucleic acids can be obtained by any suitable method known in the art, including, for example: (1) hybridization of genomic or cDNA libraries with probes to detect homologous nucleotide sequences; (2) antibody screening of expression libraries to detect cloned DNA fragments with shared structural features; (3) various amplification procedures such as polymerase chain reaction (PCR) using primers capable of annealing to the nucleic acid of interest; and (4) direct chemical synthesis.

The desired nucleic acids can also be cloned using well-known amplification techniques. Examples of protocols sufficient to direct persons of skill through in vitro amplification methods, including the polymerase chain reaction (PCR) the ligase chain reaction (LCR), Qβ-replicase amplification and other RNA polymerase mediated techniques, are found in Berger, Sambrook, and Ausubel, as well as Mullis et al. (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039.

As an alternative to cloning a nucleic acid, a suitable nucleic acid can be chemically synthesized. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method described in U.S. Pat. No. 4,458,066. Chemical synthesis produces a single stranded polynucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences can be cloned and the appropriate subsequences cleaved using appropriate restriction enzymes. The fragments can then be ligated to produce the desired DNA sequence.

C. Utility of Differentially Expressed Nucleic Acids and Expression Profiles

As alluded to above and described in greater detail below, the differentially expressed nucleic acids and expression profiles of the invention can be used as cytotoxicity markers to detect cells in a toxic state and can be used in a variety of screening and diagnostic methods. For example, the differentially expressed nucleic acids of the invention find utility as hybridization probes or amplification primers. In certain instances, these probes and primers are fragments of the differentially expressed nucleic acids of the lengths described earlier in this section. In general, such fragments are of sufficient length to specifically hybridize to an RNA or DNA in a sample obtained from a subject. Typically, the nucleic acids are 10-20 nucleotides in length, although they can be longer as described above. The probes can be used in a variety of different types of hybridization experiments, including, but not limited to, Northern blots and Southern blots and in the preparation of custom arrays (see infra). The differentially expressed nucleic acids can also be used in the design of primers for amplifying the differentially expressed nucleic acids of the invention and in the design of primers and probes for quantitative RT-PCR. Most frequently, the primers include about 20 to 30 contiguous nucleotides of the nucleic acids of the invention in order to obtain the desired level of stability and thus selectivity in amplification, although longer sequences as described above can also be utilized.

Hybridization conditions are varied according to the particular application. For applications requiring high selectivity (e.g., amplification of a particular sequence), relatively stringent conditions are utilized, such as 0.02 M to about 0.10 M NaCl at temperatures of about 50° C. to about 70° C. High stringency conditions such as these tolerate little, if any, mismatch between the probe and the template or target strand. Such conditions are useful for isolating specific genes or detecting particular mRNA transcripts, for example.

Other applications, such as substitution of amino acids by site-directed mutagenesis, require less stringency. Under these conditions, hybridization can occur even though the sequences of the probe and target are not perfectly complementary, but instead include one or more mismatches. Conditions can be rendered less stringent by increasing the salt concentration and decreasing temperature. For example, a medium stringency condition includes about 0.1 to 0.25 M NaCl at temperatures of about 37° C. to about 55° C. Low stringency conditions include about 0.15M to about 0.9 M salt, at temperatures ranging from about 20° C. to about 55° C.

V. Proteins

A. General

The differentially expressed nucleic acids of the inventions (including ESTs for which the full-length gene has been identified according to the methods described above) can be inserted into any of a number of known expression systems to generate large amounts of the protein encoded by the gene or gene fragment. Such proteins can then be utilized in the preparation of antibodies. Proteins encoded by target genes can be utilized in the compound development programs described below.

The polypeptides can be isolated from natural sources, and/or prepared according to recombinant methods, and/or prepared by chemical synthesis, and/or prepared using a combination of recombinant methods and chemical synthesis. Besides substantially full-length polypeptides, the present invention provides for biologically active fragments of the polypeptides. Biological activity can include, for example, antibody binding (e.g., the fragment competes with a full-length polypeptide) and immunogenicity (i.e., possession of epitopes that stimulate B- or T-cell responses against the fragment). Such fragments generally comprise at least 5 contiguous amino acids, typically at least 6 or 7 contiguous amino acids, in other instances 8 or 9 contiguous amino acids, usually at least 10, 11 or 12 contiguous amino acids, in still other instances at least 13 or 14 contiguous amino acids, in yet other instances at least 16 contiguous amino acids, and in some cases at least 20, 40, 60 or 80 contiguous amino acids.

Often the polypeptides of the invention will share at least one antigenic determinant in common with the amino acid sequence of the full-length polypeptide. The existence of such a common determinant is evidenced by cross-reactivity of the variant protein with any antibody prepared against the full-length polypeptide. Cross-reactivity can be tested using polyclonal sera against the full-length polypeptide, but can also be tested using one or more monoclonal antibodies against the full-length polypeptide.

The polypeptides include conservative variations of the naturally occurring polypeptides. Such variations can be minor sequence variations of the polypeptide that arise due to natural variation within the population (e.g., single nucleotide polymorphisms) or they can be homologs found in other species. They also can be sequences that do not occur naturally but that are sufficiently similar so that they function similarly and/or elicit an immune response that cross-reacts with natural forms of the polypeptide. Sequence variants can be prepared by standard site-directed mutagenesis techniques. The polypeptide variants can be substitutional, insertional or deletion variants. Deletion variants lack one or more residues of the native protein that are not essential for function or immunogenic activity (e.g., polypeptides lacking transmembrane or secretory signal sequences). Substitutional variants involve conservative substitutions or one amino acid residue for another at one or more sites within the protein and can be designed to modulate one or more properties of the polypeptide such as stability against proteolytic cleavage. Insertional variants include, for example, fusion proteins such as those used to allow rapid purification of the polypeptide and also can include hybrid proteins containing sequences from other polypeptides which are homologues of the polypeptide. The foregoing variations can be utilized to create equivalent, or even an improved, second-generation polypeptide.

The polypeptides of the invention also include those in which the polypeptide has a modified polypeptide backbone. Examples of such modifications include chemical derivatizations of polypeptides, such as acetylations and carboxylations. Modifications also include glycosylation modifications and processing variants of a typical polypeptide. Such processing steps specifically include enzymatic modifications, such as ubiquitinization and phosphorylation. See, e.g., Hershko & Ciechanover, Ann. Rev. Biochem. 51:335-364 (1982). Also included are mimetics which are peptide-containing molecules that mimic elements of protein secondary structure (see, e.g., Johnson, et al., “Peptide Turn Mimetics” in Biotechnology and Pharmacy, (Pezzuto et al., Eds.), Chapman and Hall, New York (1993)). Peptide mimetics are typically designed so that side chain groups extending from the backbone are oriented such that the side chains of the mimetic can be involved in molecular interactions similar to the interactions of the side chains in the native protein.

B. Production of Polypeptides

1. Recombinant Technologies

The polypeptides encoded by the differentially expressed nucleic acids of the invention can be expressed in hosts after the coding sequences have been operably linked to an expression control sequence in an expression vector. Expression vectors are typically replicable in the host organisms either as episomes or as an integral part of the host chromosomal DNA. Commonly, expression vectors contain selection markers, e.g., tetracycline resistance or hygromycin resistance, to permit detection and/or selection of those cells transformed with the desired DNA sequences (see, e.g., U.S. Pat. No. 4,704,362).

Typically, a differentially expressed gene of the invention is placed under the control of a promoter that is functional in the desired host cell to produce relatively large quantities of a polypeptide of the invention. An extremely wide variety of promoters are well-known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of such control sequences are termed “expression cassettes.” Accordingly, the invention provides expression cassettes into which the nucleic acids of the invention are incorporated for high level expression of the corresponding protein in a desired host cell.

In certain instances, the expression cassettes are useful for expression of polypeptides in prokaryotic host cells. Commonly used prokaryotic control sequences (defined herein to include promoters for transcription initiation, optionally with an operator, along with ribosome binding site sequences) include such commonly used promoters as the beta-lactamase (penicillinase) and lactose (lac) promoter systems (Change et al. (1977) Nature 198: 1056), the tryptophan (trp) promoter system (Goeddel et al. (1980) Nucleic Acids Res. 8: 4057), the tac promoter (DeBoer et al. (1983) Proc. Natl. Acad. Sci. U.S.A. 80:21-25); and the lambda-derived PL promoter and N-gene ribosome binding site (Shimatake et al. (1981) Nature 292: 128). In general, however, any available promoter that functions in prokaryotes can be used.

For expression of polypeptides in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli.

For expression of the polypeptides in yeast, convenient promoters include GAL1-10 (Johnson and Davies (1984) Mol. Cell. Biol. 4:1440-1448) ADH2 (Russell et al. (1983) J. Biol. Chem. 258:2674-2682), PHO5 (EMBO J. (1982) 6:675-680), and MFα (Herskowitz and Oshima (1982) in The Molecular Biology of the Yeast Saccharomyces (eds. Strathem, Jones, and Broach) Cold Spring Harbor Lab., Cold Spring Harbor, N.Y., pp. 181-209). Another suitable promoter for use in yeast is the ADH2/GAPDH hybrid promoter as described in Cousens et al., Gene 61:265-275 (1987). Other promoters suitable for use in eukaryotic host cells are well-known to those of skill in the art.

For expression of the polypeptides in mammalian cells, convenient promoters include CMV promoter (Miller, et al., BioTechniques 7:980), SV40 promoter (de la Luma, et al.,(1998) Gene 62:121), RSV promoter (Yates, et al, (1985) Nature 313:812), MMTV promoter (Lee, et al.,(1981) Nature 294:228).

For expression of the polypeptides in insect cells, the convenient promoter is from the baculovirus Autographa Californica nuclear polyhedrosis virus (NcMNPV) (Kitts, et al., (1993) Nucleic Acids Research 18:5667).

Either constitutive or regulated promoters can be used in the expression systems. Regulated promoters can be advantageous because the host cells can be grown to high densities before expression of the polypeptides is induced. High level expression of heterologous proteins slows cell growth in some situations. For E. coli and other bacterial host cells, inducible promoters include, for example, the lac promoter, the bacteriophage lambda P_{L} promoter, the hybrid trp-lac promoter (Amann et al. (1983) Gene 25: 167; de Boer et al. (1983) Proc. Nat'l. Acad. Sci. USA 80: 21), and the bacteriophage T7 promoter (Studier et al. (1986) J. Mol. Biol.; Tabor et al. (1985) Proc. Nat'l. Acad. Sci. USA 82: 1074-8). These promoters and their use are discussed in Sambrook et al., Molecular Cloning: A Laboratory Manual, Cold Spring Harbor Press, N.Y., (1989). Inducible promoters for other organisms are also well-known to those of skill in the art. These include, for example, the arabinose promoter, the lacZ promoter, the metallothionein promoter, and the heat shock promoter, as well as many others.

Construction of suitable vectors containing one or more of the above listed components employs standard ligation. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. A wide variety of cloning and in vitro amplification methods suitable for the construction of recombinant nucleic acids is described, for example, in Berger and Kimmel, Guide to Molecular Cloning Techniques, Methods in Enzymology, Volume 152, Academic Press, Inc., San Diego, Calif. (Berger); and “Current Protocols in Molecular Biology,” F. M. Ausubel et al., eds., Current Protocols, a joint venture between Greene Publishing Associates, Inc. and John Wiley & Sons, Inc., (1998 Supplement) (Ausubel).

There are a variety of suitable vectors suitable for use as starting materials for constructing the expression vectors containing the differentially expressed nucleic acids of the invention. For cloning in bacteria, common vectors include pBR322-derived vectors such as pBLUESCRIPT™, pUC18/19, and λ-phage derived vectors. In yeast, suitable vectors include Yeast Integrating plasmids (e.g., YIp5) and Yeast Replicating plasmids (the YRp series plasmids) pYES series and pGPD-2 for example. Expression in mammalian cells can be achieved, for example, using a variety of commonly available plasmids, including pSV2, pBC12BI, and p91023, pCDNA series, pCMV1, pMAMneo, as well as lytic virus vectors (e.g., vaccinia virus, adenovirus), episomal virus vectors (e.g., bovine papillomavirus), and retroviral vectors (e.g., murine retroviruses). Expression in insect cells can be achieved using a variety of baculovirus vectors, including pFastBac1, pFastBacHT series, pBluesBac4.5, pBluesBacHis series, pMelBac series, and pVL1392/1393, for example.

The polypeptides encoded by the full-length genes or fragments thereof can be expressed in a variety of host cells, including E. coli, other bacterial hosts, yeast, and various higher eukaryotic cells such as the COS, CHO, HeLa and myeloma cell lines. The host cells can be mammalian cells, plant cells, insect cells or microorganisms, such as, for example, yeast cells, bacterial cells, or fungal cells. Examples of useful bacteria include, but are not limited to, Escherichia, Enterobacter, Azotobacter, Erwinia, Klebsielia.

The expression vectors of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant polypeptides can be purified according to standard procedures of the art, including ammonium sulfate precipitation, affinity columns, ion exchange and/or size exclusivity chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Typically, the polypeptides are purified to obtain substantially pure compositions of at least about 90 to 95% homogeneity; in other applications, the polypeptides are further purified to at least 98 to 99% or more homogeneity.

2. Naturally occurring Polypeptides

Naturally occurring polypeptides encoded by the differentially expressed nucleic acids of the invention can also be isolated using conventional techniques such as affinity chromatography. For example, polyclonal or monoclonal antibodies can be raised against the polypeptide of interest and attached to a suitable affinity column by well-known techniques. See, e.g., Hudson & Hay, Practical Immunology (Blackwell Scientific Publications, Oxford, UK, 1980), Chapter 8 (incorporated by reference in its entirety). Peptide fragments can be generated from intact polypeptides by chemical or enzymatic cleavage methods known to those of skill in the art.

3. Other Methods

Alternatively, the polypeptides encoded by differentially expressed genes or gene fragments can be synthesized by chemical methods or produced by in vitro translation systems using a polynucleotide template to direct translation. Methods for chemical synthesis of polypeptides and in vitro translation are well-known in the art, and are described further by Berger & Kimmel, Methods in Enzymology, Volume 152, Guide to Molecular Cloning Techniques, Academic Press, Inc., San Diego, Calif., 1987 (incorporated by reference in its entirety).

C. Utility

The polypeptides can be used to generate antibodies that specifically bind to epitopes associated with the polypeptides or fragments thereof. Commercially available computer sequence analysis can be used to determine the location of the predicted major antigenic determinant epitopes of the polypeptide (e.g., MacVector from IBI, New Haven, Conn.). Once such an analysis has been performed, polypeptides can be prepared that contain at least the essential structural features of the antigenic determinant and can be utilized in the production of antisera against the polypeptide. Minigenes or gene fusions encoding these determinants can be constructed and inserted into expression such as those described above using standard techniques. The major antigenic determinants can also be determined empirically in which portions of the gene encoding the polypeptide are expressed in a recombinant host, and the resulting proteins tested for their ability to elicit an immune response. For example, PCR can be used to prepare a range of cDNAs encoding polypeptides lacking successively longer fragments of the C-terminus of the polypeptide. The immunoprotective activity of each of these polypeptides then identifies those fragments or domains of the polypeptide that are essential for this activity. Further experiments in which only a small number or amino acids are removed at each iteration then allows the location of the antigenic determinants of the polypeptide.

Polypeptides encoded by target genes can be utilized in the development of pharmaceutical compositions, for example, that modulate gene products associated with toxic effects. The process for identifying such polypeptides and subsequent compound development is described further below.

VI. Screening Methods—Toxicants and Antidotes

The invention provides a number of different screening methods that utilize the differentially expressed nucleic acids of the invention including, for example, screens to identify toxic compounds and screens to identify antidotes. In general, these methods involve determining the expression level of one or more of the differentially expressed nucleic acids of the invention in a test sample and then comparing the level of expression to the level of expression of the same genes in a control sample. A finding that there is a difference in the level of expression between the two samples is an indicator of a toxic response.

A. Screening Compounds to Identify Toxicants

The differentially expressed nucleic acids of the invention have value in the high throughput screening of compounds to identify toxicants. Such screens are useful in the pharmaceutical industry, for example, in rapidly screening pharmaceutical candidates for potential toxicity. If the results of the screen indicate that a lead compound exhibits toxic characteristics, derivatives can be prepared to avoid such toxic effects. Different cells or populations of cells can also be contacted with different concentrations of a potential toxicant to develop a toxicity profile or dose response for the toxicant, thereby establishing the degree of toxicity of the toxicant. The screens are also useful, for example, in screening existing or new consumer products for potential toxicity before marketing to the general public. The results of such tests can be used to identify products to which access should be restricted or identify those products for which instructions and/or warnings regarding appropriate use may be warranted.

This type of screening assay typically involves contacting a test cell or population of test cells with a potential toxicant (i.e., test compound). A control cell or population of control cells is treated similarly in a parallel reaction, except that it is not contacted with the potential toxicant. The level of expression of one or more differentially expressed nucleic acids is then determined for both the test and control cell. A difference in expression indicates that the potential toxicant is a toxicant. As described above, the difference should be a statistically significant difference.

B. Screening Compounds to Identify Antidotes

With the differentially expressed nucleic acids of the invention, screens can also be conducted to identify compounds that are antidotes to known toxicants. Such methods closely parallel the screening methods just described for identifying toxicants. However, in these assays, cells or populations of cells are initially contacted with a known toxicant at a sufficiently high concentration and for sufficient duration to induce differential expression of at least one (more typically a plurality) of the differentially expressed nucleic acids of the invention. Coincident with, or subsequent to, treatment with the known toxicant, the cell or population of cells is then contacted with a potential antidote for a sufficient period of time to allow the potential antidote the opportunity to counteract the differential expression caused by the known toxicant. The level of expression of one or more of the differentially expressed genes is then determined. A level of expression characteristic for a cell in a non-toxic state indicates that the potential antidote is in fact an antidote.

Alternatively, screens can be performed to identify compounds capable of binding to a target gene or target gene product that has been identified as being a causative agent in the formation of a toxic state in cells. Compounds capable of binding to such targets are good candidates for antidotes. Such screens are described in further detail below.

C. Contacting

The contacting step in which, for example, a potential toxicant or antidote is brought into contact with a test cell can be performed in a variety of formats known to those with skill in the art. One method, described more fully in the Examples, involves initially growing cells in culture and then transferring the cells to treatment solutions containing a desired concentration of test compound and optionally a compound to enhance uptake of the test compound. The cells are kept in contact with the test solution for a selected time period sufficient such that if the test compound is in fact a toxicant a cytotoxic response is generated. The cells are then separated from the treatment solution and RNA isolated according to the methods described above. The RNA can then be analyzed using the differential expression methods described above. In some instances, cells are grown in the treatment solution for varying periods of time to determine a time response profile. Similarly, concentrations of the test compound can be varied to determine dose responses.

Typically, cells are kept in contact with a test solution for at least a few hours but less than 24 hours. Although for tests on the effects of brief or prolonged exposures to a toxicant, the contact time can be significantly longer or shorter. The concentration of toxicant can also vary depending on the nature of the screen. In the case of screens of pharmaceutical compounds, for example, the concentration can be selected in relation to the therapeutically effective dose. For instance, the concentration can be 10, 20, 50 or 100 times the therapeutically effective dose.

Another useful format, particularly for techniques such as in situ hybridization is to place a population of test cells (generally about 10⁴ to 10⁶ in number) in the wells of one or more microtiter plates. Different test compounds can than be separately added to different wells. The test cells are then contacted with a compound for a sufficiently long period and at a sufficiently high concentration to allow for modulation of the expression of differentially expressed genes. Labeled probes that specifically hybridize to differentially expressed nucleic acids can then be added to form hybridization complexes that can be detected.

In some instances (e.g., for very high throughput screening), multiple compounds can initially be included in a treatment solution or contacted with cells in microtiter wells. For those solutions or wells showing differential expression (or a reduction in differential expression in the case of antidotes), the multiple compounds added to that particular well can then be separately assayed to identify the active compound(s). If none of the compounds when separately assayed appear capable of generating a toxic response, then this indicates that the initial toxic response was a consequence of interaction between one or more of the test compounds.

D. Determination of Differential Expression

Following the contacting step, RNA or mRNA is then typically extracted from the test cells in each of the wells according to the methods described above. Genes whose level of transcription is modulated can be identified using the probes, probe arrays and primers described above in the differential expression methods set forth earlier in the section on differential gene analysis (e.g., DD-PCR, probe arrays, quantitative RT-PCR, Northern blots, dot blots, in situ hybridization and reporter assays). The custom probe arrays and reporter assays described below can also be utilized.

The assays involve the detection of at least one differentially expressed nucleic acid of the invention. More typically, however, the assays involve detecting the differential expression of a plurality of differentially expressed nucleic acids of the invention as such expression provides more convincing evidence of an authentic toxic response. Thus, some assays involve monitoring at least 2, 3, 4, 5, 6, 7, 8, 9, 10, 12, 14, 16, 18, 20, 25, 30, 35, 40, 45 or all of the differentially expressed nucleic acids of the invention.

In some instances, certain subsets of genes are examined. For example, one subset of genes includes “stress genes” (e.g., XP-C repair complementing protein, Glutathione-S-transferase, Metallothionein-1H, Heat shock protein 90, cAMP-dependent transcription factor ATF-4 and EST (AI148382). In other instances, the subset of genes can include those that belong to the so-called group of house keeping genes involved in normal cellular activity (e.g., Cytochrome c-1, F1F₀-ATPase synthase, Ubiquinol-cytochrome c reductase core protein II, Lactate dehydrogenase-A, Pyruvate dehydrogenase E1-beta subunit and NADH dehydrogenase subunit 2). A subset of genes used in other methods includes genes involved in cellular apoptosis (e.g., Acinus and Defender against cell death 1). Certain other screening methods focus on those nucleic acids whose expression is up-regulated or down-regulated relative to controls.

E. Control Samples

Generally assays with control cells are run in parallel to the reactions with test cells. In such control screens, control cells are treated under conditions identical to those of the test cells, except that the cells are not contacted with a test compound or are contacted with a compound known not to be toxic. A difference in the level of expression for one or more of the differentially expressed genes of the invention in the test cells as compared to the control cell indicates that the compound contacted with the test cells exhibiting differential expression is a toxicant.

F. Test Compounds

The screens can be conducted with essentially any type of test compound for which toxicity information is desired or compounds having potential value as antidotes. The test compound can also be a mixture of compounds, as in some instances a mixture of compounds is toxic whereas the individual components of the mixture are not. The compounds can be organic or inorganic (e.g., metal ions).

Pharmaceutical compounds are one general class of compounds that can be screened according to the present invention. For example, the screening methods can be used to conduct toxicity tests on potential pharmaceutical compounds as part of the assessment of the relative efficacy and toxicity of the compound. In pharmaceutical screening, the test compounds can be of essentially any chemical type that can be formulated for administration to humans. Thus, test compounds include, but are not limited to, polynucleotides, polypeptides, oligosaccharides, lipids, phospholipids, heterocyclic compounds and urea based derivatives.

The methods can also be used to screen non-pharmaceutical compounds including, but not limited to, solvents, food additives, cosmetic ingredients, cleansers, preservatives, household products, dyes, personal hygiene products, pesticides, herbicides, insecticides and the like.

G. Cells

A variety of different types of cells can be utilized in such screens provided the cells are capable of expressing at least one of the differentially expressed nucleic acids of the invention. Cells can be obtained from a variety of different human tissues including, but not limited to, liver, breast, skin, kidney, stomach and pancreas. Suitable cells lines include, for example, HepG2, HeLa, HL60 and MCF7 cells.

VII. Diagnostic Methods

The differentially expressed nucleic acids of the invention can also be utilized in diagnostic applications to detect individuals suffering from a toxic condition. The general approach is similar to that described for the screening methods. In this instance, a nucleic acid sample from an individual suspected of suffering from exposure to a toxicant is obtained. The withdrawn sample is then utilized in combination with the probes, primers or probe arrays disclosed herein to detect whether one or more differentially expressed nucleic acids is in fact differentially expressed, thereby indicating that the individual is reacting to contact with a toxicant.

By using probes, primers or probe arrays that hybridize to particular sets of differentially expressed nucleic acids that are modulated for certain toxic states or in response to particular toxicants (e.g., fingerprint genes), one can more specifically identify the nature of the toxic exposure. Customized probe arrays containing specific probes for such states or toxicants are useful for such analyses. Comparison of the differential level of expression in the test individual with expression profiles specific for particular toxic states or toxicants can also be utilized to more specifically assess the nature of a toxic response.

Samples obtained from human subjects can be obtained from essentially any source from which nucleic acids can be obtained. If the toxic response effects primarily certain tissues or organs, than the sample should be obtained from such sources. In general, however, samples can be obtained from sputum, blood, tissue or fine needle biopsy samples, urine, peritoneal fluid, and fleural fluid, or cells therefrom. Biological samples can also include sections of tissues such as frozen sections taken for histological purposes.

VIII. Screening Assays—Compounds that Interact with Target Genes

Genes modulated under toxic conditions can fall into one of several categories, including for example: (1) genes whose modulation leads to toxic outcomes (e.g., inhibition of cell proliferation or apoptosis; (2) genes whose modulation results in a protective effect against the toxicant; or (3) genes that are indicative of toxicity but that are not directly involved in either the mechanism of toxicity or the cell's protective response.

Target genes and the respective target gene products are those genes and products shown to affect cytotoxicity and thus are not simply markers of a cytotoxic state (although they can be markers). A variety of assays can be designed to identify compounds that bind to target gene products, bind to other cellular or extracellular proteins that interact with a target gene product, or interfere with the interaction of the target gene product with other cellular or extracellular proteins. For example, in some instances, the expression level of a target gene product is reduced and this overall lower level of target gene expression and/or target gene product results in cytotoxicity. In such instances, screens can be developed to identify compounds that interact with the target gene or target gene product to increase the activity of the target gene or target gene product. In so doing, such compounds effectively increase the level of target gene product activity, thereby reducing the severity of the cytotoxic state.

In other instances, up-regulation of a target gene results in increased target gene product that in turn causes cytotoxicity. In this instance, screens are designed to identify compounds that interact with the target gene or gene product to decrease the activity of the target gene or gene product. Such compounds can be utilized in treatments to ameliorate the risks associated with cytotoxicity. The opposite situation also exists in which the up-regulation of a target gene yields a target gene product that exerts a protective effect that counteracts the toxic effect of a toxicant. The goal of screens in such instances is to identify compounds that enhance the expression of such up-regulated genes or the activity of their gene products, thereby reducing the severity of a cytotoxic condition.

Target genes themselves can be identified by appropriate experiments in which expression of the target gene(s) is artificially modulated independent of toxicant action. For example, genes whose up-regulation exerts a protective effect can, when cloned, transfected into test cells and expressed at high levels, reduce the degree of toxicity observed when the cells are challenged with toxicant. Similarly, for those target genes whose down-regulation exerts a positive effect, deletion of the gene can reduce the degree of toxicity observed. In like manner, the overexpression of target genes whose expression causes toxicity can exacerbate the toxic response, whereas deletion of such a gene can lessen the toxic response.

A. Assays for Compounds Capable of Binding Target Gene Product

A variety of methods can be developed to identify compounds that bind to a target gene or gene product. In certain assays, the protein encoded by the target gene is contacted with a test compound under conditions and for a sufficient period of time to allow the two components to interact and form a complex that can be isolated and/or detected in the reaction mixture. A variety of different formats known to those in the art can be utilized for conducting such binding assays.

For example, either the target gene protein or the test compound can be attached to a solid phase and then the other component added and sufficient time provided to allow for formation of a test compound/target gene protein complex. Unbound components are removed, typically by washing, under conditions that allow complexes to remain immobilized to the solid support. Detection of complexes can be achieved in various ways. If the nonimmobilized component is labeled, complexes can be detected simply by identifying immobilized label on the support. If the nonimmobilized component was not labeled prior to complex formation, complexes can be detected using indirect methods. For example, a labeled antibody with binding specificity for the initially nonimmobilized component can be added to form a complex with the initially non-immobilized component (alternatively, an unlabeled antibody can be added and than a labeled antibody having binding specificity for the unlabeled antibody added to form a labeled complex).

Binding assays can also be conducted in solution wherein the test compound and target gene protein are allowed to form complexes which can than be separated from uncomplexed components. One such approach includes immobilizing an antibody specific for the target gene product (or less frequently the test compound) which in turn immobilizes the complex to the support. By labeling one of the components immobilized complexes can be detected.

B. Assays for Compounds that Interfere with the Interaction between Target Gene Products and Other Compounds

In exerting their in vivo effect, target proteins can interact with one or more cellular or extracellular proteins to form complexes. The proteins in such complexes are referred to as binding partners. Compounds capable of disrupting the interaction between such partners can be useful in regulating the activity of the target gene proteins.

Numerous assays can be conducted to disrupt the interaction between the binding partners. One approach involves contacting the target gene product with a its binding partner both in the presence and absence of a test compound. The test compound can be included at the time the binding partners are contacted, or can be added sometime subsequent to mixing the binding partners together. Parallel control experiments are conducted under identical conditions, except that the test compound is not included in the control mixture or a control compound known not to influence the binding of the partners is included in the mixture. Formation of complexes between the partners is then detected. The formation of complexes in the control reaction mixture but not in the test mixture indicates that the test compound interferes with the interaction between the binding partners. Such assays can be conducted in heterogeneous assays in which one of the binding members is immobilized to a solid support or in homogeneous assays in which all components are contacted with one another in the liquid phase using methods similar to those set forth in the preceding section.

IX. Compounds for Inhibiting or Enhancing the Synthesis or Activity of Target Genes

A. Activity or Synthesis Inhibition

As discussed above, certain target genes can cause or worsen cytotoxicity when up-regulated in response to a toxic insult. The increase in the activity of such target genes and their products can be countered using various methodologies to inhibit the expression, synthesis or activity of such target genes and/or proteins.

For example, antisense, ribozyme, triple helix molecules and antibodies can be utilized to ameliorate the negative effects of such target genes and gene products. Antisense RNA and DNA molecules act directly to block the translation of mRNA by hybridizing to targeted mRNA, thereby blocking protein translation. Hence, a useful target for antisense molecules is the translation initiation region.

Ribozymes are enzymatic RNA molecules that hybridize to specific sequences and then carry out a specific endonucleolytic cleavage reaction. Thus, for effective use, the ribozyme should include sequences that are complementary to the target mRNA, as well as the sequence necessary for carrying the cleavage reaction (see, e.g., U.S. Pat. No. 5,093,246).

Nucleic acids utilized to promote triple helix formation to inhibit transcription are single-stranded and composed of dideoxyribonucleotides. The base composition of such polynucleotides is designed to promote triple helix formation via Hoogsteen base pairing rules and typically require significant stretches of either pyrimidines or purines on one strand of a duplex.

Antibodies having binding specificity for a target gene protein that also interferes with the activity of the gene protein can also be utilized to inhibit gene protein activity. Such antibodies can be generated from full-length proteins or fragments thereof according to the methods described below.

B. Activity Enhancement

Cytotoxicity can be exacerbated by underexpression of certain target genes and/or by a reduction in activity of a target gene product. Alternatively, the up-regulation of certain target gene products can produce a beneficial effect. In any of these scenarios, it is useful to increase the expression, synthesis or activity of such target genes and proteins.

These goals can be achieved, for example, by increasing the level of target gene product or the concentration of active gene product. Hence, in one approach, a target gene protein in the form of a pharmaceutical composition such as that described below is administered to a subject suffering from toxicity. Alternatively, RNA sequences encoding target gene proteins can be administered to a patient at a concentration sufficient to lessen the severity of the cytoxic condition, again according to methods such as those described below. Gene therapy is yet another option and includes inserting one or more copies of a normal target gene, or a fragment thereof capable of producing a functional target protein, into cells using various vectors. Suitable vectors include, for example, adenovirus, adeno-associated virus and retrovirus vectors. Liposomes and other particles capable of introducing DNA into cells can also be utilized in some instances. Cells, typically autologous cells, that express a normal target gene can than be introduced or reintroduced into a patient to lessen the effects of cytotoxicity.

X. Identification of Pathway Genes

Pathway genes are genes whose expression product is capable of interacting with gene products associated with cellular toxicity. In some instances, pathway genes are differentially expressed and can have the characteristics of a fingerprint gene and/or a target gene.

A variety of different methods can be utilized to identify pathway genes. In general, such methods typically are capable of detecting protein/protein interactions, as such methods can be used to identify interactions between gene products and the gene products known to be associated with cytotoxicity. Such known gene products can be cellular or extracellular proteins. Those gene products that interact which such known genes are pathway gene products and the genes encoding them are pathway genes.

Suitable methods include, but are not limited to, co-immunoprecipitation, crosslinking and co-purification via gradients or standard chromatographic methods, for example. Once identified, a pathway gene product can be utilized to identify its corresponding pathway gene according to a variety of known methods. For example, at least a portion of the amino acid sequence of the pathway gene product can be determined by Edman degradation (see, e.g., Creighton, Proteins: Structures and Molecular Principles, W. Freeman and Co., N.Y., pp. 34-49 (1983)). The amino acid sequence so obtained can then be utilized as a guide for the preparation of polynucleotide mixtures that can be used to screen for pathway gene sequences. Screening can be accomplished, for example, using known hybridization or PCR techniques. (See, e.g., Current Protocols in Molecular Biology, (Ausbel, F. M. et al., Eds.), John Wiley & Sons, Inc., New York (1987-1993); and PCR Protocols: A Guide to Methods and Applications, (Innis, M. et al., Eds.), Academic Press, Inc., New York (1990)).

Furthermore, certain methods can be utilized to simultaneously identify pathway genes that encode a protein that interacts with a protein involved in cytotoxicity. Such methods include, for example, probing expression libraries with a labeled protein known or suggested to be involved in the formation of cellular toxicity. Another set of methods useful for the identification of protein interactions in vivo include the so-called “two hybrid systems.” A variety of such methods have been developed to screen a library of genes encoding a gene product capable of interacting with a protein of interest. See, for example, Chien et al., Proc. Natl. Acad. Sci. USA 88:9578-9582 (1991); Bartel, et al., Methods Enzymology 254:241-263 (1995); and Gietz, et al., Molecular and Cellular Biochemistry 172:67-79 (1997), each of which is incorporated by reference in its entirety. Kits for conducting such analyses are available from various commercial sources including Clontech (Palo Alto, Calif.).

XI. Characterization of Differentially Expressed Genes and Pathway Genes

The differentially expressed nucleic acids of the invention and the pathway genes identified according to the methods set forth in the previous section can be further characterized to obtain information regarding the particular biological function of the genes generally and in cytotoxic response specifically. Such an assessment can permit the genes to be designated as being target and/or fingerprint genes, for example. More specifically, as described above, any of the differentially expressed nucleic acids of the invention which upon further characterization indicate that a modulation of the gene's expression or a modulation of the gene product's activity can lessen cytotoxicity are designated target genes. Such target genes and their corresponding gene products can serve as targets for compounds whose interaction with the target gene or gene product ameliorates cytotoxicity. As also noted above, differentially expressed genes that are not necessarily causative agents of cytotoxicity but whose expression contributes to a gene expression pattern that correlates with cellular toxicity can be assigned as fingerprint genes. In like manner, analysis of pathway genes can show that certain pathway genes are in fact target genes and/or fingerprint genes.

One characterization method involves analyzing the tissue distribution of the mRNA produced by the differentially expressed or pathway genes. Techniques for conducting such analyses include, for example, Northern analyses and RT-PCR. Such analyses can provide information as to whether the differentially expressed or pathway genes are expressed in tissues particularly sensitive to toxic effects, for example.

The differentially expressed and pathway genes can be further analyzed by conducting time course experiments to determine the level of differential expression over time. As described more fully in the Examples below, in some, if not many, instances, there are temporal patterns of expression among genes affected by toxic treatments. If expression profiling is conducted at only a single time point, there is a risk of failing to identify the full set of genes affected. Furthermore, by requiring a statistically significant change in expression at several different time points, one lessens the risk of including in the set of differentially expressed genes those which undergo only transient changes in the level of expression for reasons unrelated to a treatment with a toxin. Thus, in general time course analysis can prove important in correctly identifying authentic differentially expressed and pathway genes and can aid in highlighting those genes that may play particularly critical roles in cytotoxic response.

The temporal response of differentially expressed genes and pathway genes can be analyzed further by conducting cluster analysis (see Example 2) to classify genes based upon their temporal patterns of differential expression. The patterns can be distinguished according to various criteria including, for example, whether the genes are up-regulated or down-regulated, the time at which modulation in expression occurs and how long the change persists. Using cluster analysis, one can identify genes that are positively correlated (e.g., the genes are up-regulated or down-regulated in a similar fashion) or negatively correlated (e.g., the expression of the genes moves in opposing directions). A positive correlation between genes can indicate, for example, that the genes may be responding to a common toxic mechanism of action.

XII. Antibodies

In another embodiment of the invention, antibodies that are immunoreactive with polypeptides expressed from the differentially expressed genes or gene fragments are provided, as are antibodies to proteins encoded by pathway genes and target genes. The antibodies can be polyclonal antibodies, distinct monoclonal antibodies or pooled monoclonal antibodies with different epitopic specificities.

A. Production of Antibodies

The antibodies of the invention can be prepared using intact polypeptide or fragments containing antigenic determinants from proteins encoded by differentially expressed genes, pathway genes or target genes as the immunizing antigen. The polypeptide used to immunize an animal can be from natural sources, derived from translated cDNA, or prepared by chemical synthesis and can be conjugated with a carrier protein. Commonly used carriers include keyhole limpet hemocyanin (KLH), thyroglobulin, bovine serum albumin (BSA), and tetanus toxoid. The coupled peptide is then used to immunize the animal (e.g., a mouse, a rat, or a rabbit). Various adjuvants can be utilized to increase the immunological response, depending on the host species and include, but are not limited to, Freund's (complete and incomplete), mineral gels such as aluminum hydroxide, surface actives substances such as lysolecithin, pluronic polyols, polyanions, peptides, oil emulsions, dinitrophenol and carrier proteins, as well as human adjuvants such as BCG (bacille Calmette-Guerin) and Corynebacterium parvum.

Monoclonal antibodies can be made from antigen-containing fragments of the protein by the hybridoma technique, for example, of Kohler and Milstein (Nature, 256:495-497, (1975); and U.S. Pat. No. 4,376,110, incorporated by reference in their entirety). See also, Harlow & Lane, Antibodies, A Laboratory Manual (C.S.H.P., NY, 1988), incorporated by reference in its entirety. The antibodies can be of any immunoglobulin class including IgG, IgM, IgE, IgA, IgD and any subclass thereof.

Techniques for generation of human monoclonal antibodies have also been described, including for example the human B-cell hybridoma technique (Kosbor et al., Immunology Today 4:72 (1983), incorporated by reference in its entirety); for a review, see also, Larrick et al., U.S. Pat. No. 5,001,065, (incorporated by reference in its entirety). An alternative approach is the generation of humanized antibodies by linking the complementarity-determining regions or CDR regions (see, e.g., Kabat et al., “Sequences of Proteins of Immunological Interest,” U.S. Dept. of Health and Human Services, (1987); and Chothia et al., J. Mol. Biol. 196:901-917 (1987)) of non-human antibodies to human constant regions by recombinant DNA techniques. See Queen et al., Proc. Natl. Acad. Sci. USA 86:10029-10033 (1989) and WO 90/07861 (incorporated by reference in its entirety). Alternatively, one can isolate DNA sequences which encode a human monoclonal antibody or a binding fragment thereof by screening a DNA library from human B cells according to the general protocol set forth by Huse et al., Science 246:1275-1281 (1989) and then cloning and amplifying the sequences which encode the antibody (or binding fragment) of the desired specificity. The protocol described by Huse is rendered more efficient in combination with phage display technology. See, e.g., Dower et al., WO 91/17271 and McCafferty et al., WO 92/01047 (each of which is incorporated by reference). Phage display technology can also be used to mutagenize CDR regions of antibodies previously shown to have affinity for the peptides of the present invention. Antibodies having improved binding affinity are selected.

Techniques developed for the production of “chimeric antibodies” by splicing the genes from a mouse antibody molecule of appropriate antigen specificity together with genes from human antibody molecule of appropriate antigen specificity can be used. A chimeric antibody is a molecule in which different portions are derived from different species, such as those having a variable region derived from a murine monoclonal antibody and a human immunoglobulin constant region. Single chain antibodies specific for the differentially expressed gene products of the invention can be produced according to established methodologies (see, e.g., U.S. Pat. No. 4,946,778; Bird, Science 242:423-426 (1988); Huston et al., Proc. Natl. Acad. Sci. USA 85:5879-5883 (1988); and Ward et al., Nature 334:544-546 (1989), each of which is incorporated by reference in its entirety). Single chain antibodies are formed by linking the heavy and light chain fragments of the Fv region via an amino acid bridge, resulting in a single chain polypeptide.

Antibodies can be further purified, for example, by binding to and elution from a support to which the polypeptide or a peptide to which the antibodies were raised is bound. A variety of other techniques known in the art can also be used to purify polyclonal or monoclonal antibodies (see, e.g., Coligan, et al., Unit 9, Current Protocols in Immunology, Wiley Interscience, (1994), incorporated herein by reference in its entirety).

Anti-idiotype technology can also be utilized in some instances to produce monoclonal antibodies that mimic an epitope. For example, an anti-idiotypic monoclonal antibody made to a first monoclonal antibody will have a binding domain in the hypervariable region that is the “image” of the epitope bound by the first monoclonal antibody.

B. Use of Antibodies

The antibodies of the invention are useful, for example, in screening cDNA expression libraries and for identifying clones containing cDNA inserts which encode structurally-related, immunocrossreactive proteins. See, for example, Aruffo & Seed, Proc. Natl. Acad. Sci. USA 84:8573-8577 (1977) (incorporated by reference in its entirety). Antibodies are also useful to identify and/or purify immunocrossreactive proteins that are structurally related to native polypeptide or to fragments thereof used to generate the antibody.

The antibodies can also be used in the detection of differentially expressed genes, such as target and fingerprint gene products, as well as pathway gene products. Thus, the antibodies can be used to detect such gene products in specific cells, tissues or serum, for example, and have utility in diagnostic assays. Various diagnostic assays can be utilized, including but not limited to, competitive binding assays, direct or indirect sandwich assays and immunoprecipitation assays (see, e.g., Monoclonal Antibodies: A Manual of Techniques, CRC Press, Inc. (1987) pp. 147-158). When utilized in diagnostic assays, the antibodies are typically labeled with a detectable moiety. The label can be any molecule capable of producing, either directly or indirectly, a detectable signal. Suitable labels include, for example, radioisotopes (e.g., ³H, ¹⁴C, ³²P, ³⁵S, ¹²⁵I), fluorophores (e.g., fluorescein and rhodamine dyes and derivatives thereof), chromophores, chemiluminescent molecules, an enzyme substrate (including the enzymes luciferase, alkaline phosphatase, beta-galactosidase and horse radish peroxidase, for example).

As noted above, antibodies are useful in inhibiting the expression products of the differentially expressed nucleic acids and are valuable in inhibiting the action of certain target gene products (e.g., target gene products identified as causing or exacerbating cytotoxicity). Hence, the antibodies also find utility in a variety of therapeutic applications.

XIII. Pharmaceutical Compositions

Compounds identified during the various screening methods that either inhibit or enhance the activity of differentially expressed gene products such as target genes products can be formulated into pharmaceutical compositions for therapeutic use. For example, compounds that inhibit target gene products associated with causing toxicity (e.g., antibodies, antisense sequences, ribozymes, triple helix molecules) can be utilized in preparing pharmaceutical compositions. Alternatively, compounds identified during screening that enhance the concentration or activity of target gene products that exert a positive effect can be incorporated into pharmaceutical compositions.

A. Composition

The pharmaceutical compositions used for treatment of cytotoxicity comprise an active ingredient such as the inhibitory and activity-enhancing compounds just described and, optionally, various other components.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, detergents and the like.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include the production of sulfate, gluconate, citrate, phosphate and the like. The polypeptides of the composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

B. Dosage

The pharmaceutical compositions can be administered for prophylactic and/or therapeutic treatments. The active ingredient in the pharmaceutical compositions typically is present in a therapeutic amount, which is an amount sufficient to remedy a toxic state or toxic symptoms associated with exposure to a toxicant. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

In prophylactic applications, compositions containing the compounds of the invention are administered to a patient susceptible to or otherwise at risk of being subjected to a potentially toxic environment. Such an amount is defined to be a “prophylactically effective” amount or dose. In this use, the precise amounts depends again on the patient's state of health and weight. Typically, the dose ranges from about 1 to 500 mg of purified protein per kilogram of body weight, with dosages of from about 5 to 100 mg per kilogram being more commonly utilized.

C. Administration

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which consist of the packaged active ingredient with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Formulations for injection can be presented in unit dosage form, e.g., in ampules or in multidose containers, with an added preservative. The compositions are formulated as sterile, substantially isotonic and in full compliance with all Good Manufacturing Practice (GMP) regulations of the U.S. Food and Drug Administration.

XIV. Development of Assays for Toxicant Induced Differential Expression

A. Customized Probe Arrays

1. Probes for Target Nucleic Acids

The differentially expressed nucleic acids of the invention can be utilized to prepare custom probe arrays for use in screening and diagnostic applications. In general, such arrays include probes such as those described above in the section on differentially expressed nucleic acids, and thus include probes complementary to full-length differentially expressed nucleic acids (e.g., cDNA arrays) and shorter probes that are typically 10-30 nucleotides long (e.g., synthesized arrays). Typically, the arrays include probes capable of detecting a plurality of the differentially expressed nucleic acids of the invention. For example, such arrays generally include probes for detecting at least 2, 3, 4, 5, 6, 7, 8, 9 or 10 differentially expressed nucleic acids. For more complete analysis, the arrays can include probes for detecting at least 12, 14, 16, 18 or 20 differentially expressed nucleic acids. In still other instances, the arrays include probes for detecting at least 25, 30, 35, 40, 45 or all the differentially expressed nucleic acids of the invention.

2. Control Probes

(a) Normalization Controls

Normalization control probes are typically perfectly complementary to one or more labeled reference polynucleotides that are added to the nucleic acid sample. The signals obtained from the normalization controls after hybridization provide a control for variations in hybridization conditions, label intensity, reading and analyzing efficiency and other factors that can cause the signal of a perfect hybridization to vary between arrays. Signals (e.g., fluorescence intensity) read from all other probes in the array can be divided by the signal (e.g., fluorescence intensity) from the control probes thereby normalizing the measurements.

Virtually any probe can serve as a normalization control. However, hybridization efficiency can vary with base composition and probe length. Normalization probes can be selected to reflect the average length of the other probes present in the array, however, they can also be selected to cover a range of lengths. The normalization control(s) can also be selected to reflect the (average) base composition of the other probes in the array. Normalization probes can be localized at any position in the array or at multiple positions throughout the array to control for spatial variation in hybridization efficiently.

(b) Mismatch Controls

Mismatch control probes can also be provided; such probes function for expression level controls or for normalization controls. Mismatch control probes are typically employed in customized arrays containing probes matched to known mRNA species. For example, certain arrays contain a mismatch probe corresponding to each match probe. The mismatch probe is the same as its corresponding match probe except for at least one position of mismatch. A mismatched base is a base selected so that it is not complementary to the corresponding base in the target sequence to which the probe can otherwise specifically hybridize. One or more mismatches are selected such that under appropriate hybridization conditions (e.g. stringent conditions) the test or control probe can be expected to hybridize with its target sequence, but the mismatch probe cannot hybridize (or can hybridize to a significantly lesser extent). Mismatch probes can contain a central mismatch. Thus, for example, where a probe is a 20 mer, a corresponding mismatch probe can have the identical sequence except for a single base mismatch (e.g., substituting a G, a C or a T for an A) at any of positions 6 through 14 (the central mismatch).

(c) Sample Preparation, Amplification, and Quantitation Controls

Arrays can also include sample preparation/amplification control probes. Such probes can be complementary to subsequences of control genes selected because they do not normally occur in the nucleic acids of the particular biological sample being assayed. Suitable sample preparation/amplification control probes can include, for example, probes to bacterial genes (e.g., Bio B) where the sample in question is a biological sample from a eukaryote.

The RNA sample can then be spiked with a known amount of the nucleic acid to which the sample preparation/amplification control probe is complementary before processing. Quantification of the hybridization of the sample preparation/amplification control probe provides a measure of alteration in the abundance of the nucleic acids caused by processing steps. Quantitation controls are similar. Typically, such controls involve combining a control nucleic acid with the sample nucleic acid(s) in a known amount prior to hybridization. They are useful to provide a quantitation reference and permit determination of a standard curve for quantifying hybridization amounts (concentrations).

3. Array Synthesis

Nucleic acid arrays for use in the present invention can be prepared in two general ways. One approach involves binding DNA from genomic or cDNA libraries to some type of solid support, such as glass for example. (See, e.g., Meier-Ewart, et al., Nature 361:375-376 (1993); Nguyen, C. et al., Genomics 29:207-216 (1995); Zhao, N. et al., Gene, 158:207-213 (1995); Takahashi, N., et al., Gene 164:219-227 (1995); Schena, et al., Science 270:467-470 (1995); Southern et al., Nature Genetics Supplement 21:5-9 (1999); and Cheung, et al., Nature Genetics Supplement 21:15-19 (1999), each of which is incorporated herein in its entirety for all purposes.)

The second general approach involves the synthesis of nucleic acid probes. One method involves synthesis of the probes according to standard automated techniques and then post-synthetic attachment of the probes to a support. See for example, Beaucage, Tetrahedron Lett., 22:1859-1862 (1981) and Needham-VanDevanter, et al., Nucleic Acids Res., 12:6159-6168 (1984), each of which is incorporated herein by reference in its entirety. A second broad category is the so-called “spatially directed” polynucleotide synthesis approach. Methods falling within this category further include, by way of illustration and not limitation, light-directed polynucleotide synthesis, microlithography, application by ink jet, microchannel deposition to specific locations and sequestration by physical barriers.

Light-directed combinatorial methods for preparing nucleic acid probes are described in U.S. Pat. Nos. 5,143,854 and 5,424,186 and 5,744,305; PCT patent publication Nos. WO 90/15070 and 92/10092; EP 476,014; Fodor et al., Science 251:767-777 (1991); Fodor, et al., Nature 364:555-556 (1993); and Lipshutz, et al., Nature Genetics Supplement 21:20-24 (1999), each of which is incorporated herein by reference in its entirety. These methods entail the use of light to direct the synthesis of polynucleotide probes in high-density, miniaturized arrays. Algorithms for the design of masks to reduce the number of synthesis cycles are described by Hubbel et al., U.S. Pat. Nos. 5,571,639 and 5,593,839, and by, Fodor et al., Science 251:767-777 (1991), each of which is incorporated herein by reference in its entirety.

Other combinatorial methods that can be used to prepare arrays for use in the current invention include spotting reagents on the support using ink jet printers. See Pease et al., EP 728, 520, and Blanchard, et al. Biosensors and Bioelectronics II: 687-690 (1996), which are incorporated herein by reference in their entirety. Arrays can also be synthesized utilizing combinatorial chemistry by utilizing mechanically constrained flowpaths or microchannels to deliver monomers to cells of a support. See Winkler et al., EP 624,059; WO 93/09668; and U.S. Pat. No. 5,885,837, each of which is incorporated herein by reference in its entirety.

4. Array Supports

Supports can be made of any of a number of materials that are capable of supporting a plurality of probes and compatible with the stringency wash solutions, Examples of suitable materials include, for example, glass, silica, plastic, nylon or nitrocellulose. Supports are generally are rigid and have a planar surface. Supports typically have from 1-10,000,000 discrete spatially addressable regions, or cells. Supports having 10-1,000,000 or 100-100,000 or 1000-100,000 cells are common. The density of cells is typically at least 1000, 10,000, 100,000 or 1,000,000 cells within a square centimeter. Each cell includes at least one probe; more frequently, the various cells include multiple probes. In general each cell contains a single type of probe, at least to the degree of purity obtainable by synthesis methods, although in other instances some or all of the cells include different types of probes. Further description of array design is set forth in WO 95/11995, EP 717,113 and WO 97/29212, which are incorporated by reference in their entirety.

B. Reporter Assays

Knowledge of the differentially expressed arrays of the invention can also be used to design reporter assay systems. In these systems, promoters or response elements from a differentially expressed gene of the invention is operably linked to a heterologous reporter gene to form a reporter construct that can be used to transfect test cells. When such cells are contacted with appropriate toxicants, the toxicant induces the transcription of the reporter, thereby generating a detectable signal. A test cell can harbor a single reporter construct or a plurality of different reporter constructs, each construct including a different promoter for activating the transcription of a different differentially expressed nucleic acid of the invention. Typically, the reporter assays utilize at least 2 or 3 different constructs so that the expression level of at least 2 or 3 different differentially expressed nucleic acids are probed. However, more constructs can be utilized, including for example, 4, 6, 8, 10, 20, 30, 40 or more, each construct including a promoter or response element from a different differentially expressed nucleic acid of the invention.

1. Promoters/Response Elements

The promoters and response elements utilized in reporter assays are responsive to selected toxicants such that a when a cell harboring a reporter construct is contacted with the toxicant(s), the promoter or response element activates transcription of the operably linked reporter gene. A response element refers to nucleic acid sequences which in combination with an operably linked minimal promoter can activate the transcription of the reporter gene.

Promoters that activate transcription of the differentially expressed nucleic acids of the invention can be prepared according to known techniques. For example, if a genomic fragment containing a promoter for one of the differentially expressed genes of the invention has been isolated or cloned into a vector, the promoter is removed using appropriate restriction enzymes. Fragments containing the promoter are then isolated and operably linked to a reporter gene that encodes a detectable product. Typically, the resulting reporter construct is ligated into a vector, the vector typically containing a selectable marker for identifying stable transfectants. Functional fusions can be assayed for by exposing transfectants to toxicants known to induce the specific promoter incorporated into the test cell and assaying for detectable product corresponding to transcription of the reporter gene.

If the nucleotide sequence of a desired promoter is known, the PCR methods can be used to amplify the promoter sequence. For example, primers that are complementary to the 5′ and 3′ ends of the desired promoter portion of the gene are synthesized. These primers are hybridized to denatured total DNA under suitable conditions and PCR reactions performed to yield clonable quantities of the desired promoter sequence. This promoter can than be operatively linked to a reporter gene to yield a reporter construct as described above.

Response elements which are responsive to a toxicant and activate a differentially expressed nucleic acid can often be synthesized using standard nucleotide synthesis techniques (e.g., polynucleotide synthesizers), since the response elements are relatively small. Polynucleotides corresponding to both strands of the response element are synthesized, annealed together and cloned into a plasmid containing a reporter gene under the control of a minimal promoter (e.g., minimal CMV promoters; see, e.g., Boshart et al., Cell 41:521-530 (1985) and U.S. Pat. No. 5,859,310).

2. Reporters

Reporter expression can be directly detected by detecting formation of transcript or of translation product using known techniques. For example, transcription product can be detected using Northern blots and the formation of certain proteins can be detected using a characteristic stain or by detecting an inherent characteristic of the protein. More typically, however, expression of reporter is determined by detecting a product formed as a consequence of an activity of the reporter. In such instances, detection of reporter expression is indirect.

Reporters that have an inherent characteristic that can be directly detected include GFP (green fluorescent protein). Fluorescence generated from this protein can be detected using a variety of commercially available fluorescent detection systems, including a FACS system for example.

Often the reporter is an enzyme that catalyzes the formation of a detectable product. Suitable enzymes include, but are not limited to, proteases, nucleases, lipases, phosphatases, sugar hydrolases and esterases. Typically, the reporter encodes an enzyme whose substrates are substantially impermeable to eukaryotic plasma membranes, thus making it possible to tightly control signal formation. Examples of suitable reporter genes that encode enzymes include, for example, β-glucuronidase, CAT (chloramphenicol acetyl transferase; Alton and Vapnek (1979) Nature 282:864-869), luciferase (lux), β-galactosidase and alkaline phosphatase (Toh, et al. (1980) Eur. J. Biochem. 182:231-238; and Hall et al. (1983) J. Mol. Appl. Gen. 2:101), each of which incorporated herein by reference.

A number of different luciferases are known and useful in the present invention. Firefly luciferase is particularly suitable (see, for example, deWet (1986) Methods in Enzymology 133:3-14; deWet et al., (1985) Proc. Natl. Acad. Sci. 82:7870-7873; deWet et al. (1987) Mol. Cell. Biol. 7:725-737, each of which is incorporated by reference). Four species of firefly from which the DNA encoding luciferase can be derived include: the Japanese GENJI and HEIKE fireflies, Luciola cruciata and Luciola lateralis; the East European firefly, Luciola mingrelica; and the North American firefly, Photinus pyralis (commercially available from Promega as the plasmid pGEM). The glow-worm Lampyris noctiluca is a further source of luciferase, having 84% sequence identity to that of Photinus pyralis.

In some instances, the reporter is part of a cascade. For example, the reporter can activate the expression of a second reporter, which can activate yet another reporter, and so on. Such reporter schemes have been described, for example, in PCT publication WO 98/25146, which is incorporated herein by reference.

Assays can be conducted using cells that include single reporter constructs, each cell containing a construct that has a different promoter. In such instances, the reporter can be the same so that it is only necessary to perform a single type of assay. If a cell contains multiple reporter constructs that have different promoters, than the reporter genes in the different constructs differ so that the identity of the promoter activated during the assay can be determined.

C. Cells

A variety of human cell types can be utilized in reporter assays. For example, the cells can come from essentially any body tissue including, but not limited to, liver, breast, skin, pancreas and stomach. Specific examples of suitable cell lines include HepG2 cells, HL60 cells, HeLa cells and MCF7 cells. Typically, the cells harbor a single reporter construct; however, as just noted, in some instances the cells harbor multiple reporter constructs that have different promoters.

Kits

Kits containing components necessary to conduct the screening and diagnostic methods of the invention are also provided by the invention. For example, certain kits typically include a plurality of probes that hybridize under stringent conditions to different differentially expressed nucleic acids of the invention. Other kits include a plurality of different primer pairs, each pair selected to effectively prime the amplification of a different differentially expressed nucleic acid of the invention. In the case when the kit includes probes for use in quantitative RT-PCR, the probes can be labeled with the requisite donor and acceptor dyes, or these can be included in the kit as separate components for use in preparing labeled probes.

The kits can also include enzymes for conducting amplification reactions such as various polymerases (e.g., RT and Taq), as well as deoxynucleotides and buffers. Cells capable of expressing one or more of the differentially expressed nucleic acids of the invention can also be included in certain kits.

Typically, the different components of the kit are stored in separate containers. Instructions for use of the components to conduct a toxicity analysis are also generally included.

The following examples are offered to illustrate, but no to limit the claimed invention.

### EXAMPLE 1

### Differential Gene Expression in Response to the Toxicants Acetaminophen, Caffeine and Thioacetamide as Determined by Differential Display PCR and Dot Blot Analyses

This set of experiments was designed to utilize differential display PCR (DD-PCR) (see e.g., Liang and Pardee, Science 257:967-971 (1992)) and dot blot assays to study gene expression changes in the HepG2 human liver cell line in response to three toxicants: acetaminophen, caffeine and thioacetamide. These particular toxicants were selected for analysis because their mechanisms of toxicity have been studied and found to vary including, mitochondrial disruption, macromolecular binding (e.g., covalent adduct between nucleic acid and/or protein and the toxicant or reactive intermediate), genotoxicity (DNA alterations), interference with calcium homeostatsis and lipid peroxidation (see e.g., Möller and Dargel, Acta pharmacol. et toxicol 55: 126-132 (1984); Burcham and Harman, Toxicology Letters 50:37-48 (1990); Burcham and Harman, J. Biol. Chem. 266:5049-5054 (1991); D'Ambrosio, Regulatory toxicology and pharmacology 19:243-281 (1994); and Casarett and Doull's Toxicology: The Basic Science of Poisons, (Klaasen, C. D., Ed.), McGraw-Hill, New York, (1996)). A goal for this set of experiments was to characterize the nature and magnitude of transcriptional changes that occur during toxic challenge, and to test whether common patterns of gene expression result from different toxic treatments.

This particular investigation utilized DD-PCR because the method makes no prior assumptions concerning which genes are important. As a result, previously unidentified genes can be revealed in DD-PCR experiments. In addition, profiles of expression changes can be readily created by using the same primer-pairs for a range of treatment conditions. Such detailed expression profiles can provide transcriptional “fingerprints” of toxic compounds, providing a better understanding of toxic mechanisms and cellular responses to injury. Lastly, the techniques and reagents are common to most molecular biology laboratories.

To avoid the possibility of false-positives (see, e.g., Debouck, Current Opinion in Biotechnology 6:597-599 (1995)), a strategy based on cycle sequencing of re-amplified DD bands followed by a rapid secondary dot blot assay to test candidate genes in an independent format was utilized to confirm the DD-PCR results. Different PCR primer pairs for each compound in the study were used to increase genome coverage; all candidate genes were subsequently tested against all treatments in the secondary assay. This approach yielded 38 genes whose expression was modulated, including nine that change in common across all three treatments.

I. Materials and Methods

A. Cell Culture and Assay

Culturing.

HepG2 cells (see e.g., Aden et al., Nature 282:615-616 (1979)) (ATCC HB-8065) were maintained in DMEM/F-12 medium with 10% fetal bovine serum and 1% antibiotic/antimycotic. For routine culturing and mRNA preps, cells were grown in 75 cm² flasks and split every 4-5 days. For plate assays, cells were plated in 96-well microtiter plates at 1×10⁵ cells per well in 100 μl of growth medium.

Cell Treatments.

Depending on the desired exposure time, cell treatments began 3 or 4 days after splitting or plating. At this time, the cells were near or at confluency. Treatment solutions were freshly prepared in serum-free medium with 0.2% DMSO added for compound solubility. Cell treatments were at 37° C.

Cell Proliferation Assays.

Uptake of 5-bromo-2′-deoxyuridine (BrdU) was measured using the Cell Proliferation ELISA kit from Boehringer-Mannheim (Indianapolis, Ind.).

Oligo(dT) Assay for Quantitation of mRNA.

This method is described in greater detail in Example 2. Briefly, after growth and treatment in 96-well plates, HepG2 cells were fixed and permeabilized with formaldehyde and Triton X-100, respectively. 5′ biotinylated poly(dT)₁₅ (Keystone Labs) was added to the wells and hybridized overnight. After washing, horseradish peroxidase-conjugated streptavidin was added, and the amount of poly(dT)₁₅ bound to the cells was quantitated spectrophotometrically after addition of TMB substrate.

B. Preparation of mRNA

Following cell lysis in guanidinium thiocyanate, mRNA was isolated by affinity purification on oligo(dT) cellulose using the Ambion Poly(A) Pure kit. Samples were aliquoted and stored at −80° C.

C. Differential display-PCR

Reagents.

Primers for differential display-PCR were obtained from Genomyx Corporation (Foster City, Calif.) as components of their HIEROGLYPH™ mRNA Profile Kit. The sequences of the 6 anchored and 17 arbitrary primers used are shown in Table 4.

Superscript II Reverse Transcriptase, dithiothreitol (DTT) and First Strand Buffer (5×) were purchased from Gibco BRL Products. AmpliTaq DNA Polymerase and 10× PCR Buffer II (containing 15 mM MgCl₂) was purchased from Perkin-Elmer (Foster City, Calif., USA). Ribonuclease Inhibitor was obtained from Ambion, Inc. or Promega Corporation (Madison, Wis., USA). Redivue [α-³³P]dATP (1000-3000 Ci/mmole specific activity) was obtained from Amersham (Arlington Heights, Ill., USA). All reactions were performed on an MJ Research PTC-100 Thermocycler, using 0.2 mL thin-walled MicroAmp PCR tubes and caps (Perkin-Elmer). Stop solution (95% formamide, 200 mM EDTA, 0.05% bromophenol blue, 0.05% xylene cyanol FF) was obtained from Amersham. The GenomyxLR gel running and drying apparatus, as well as plates, combs, 340 micron-thick spacers, 4.5% acrylamide denaturing gel mix, and dNTP mixture (250 μM each: dATP, dCTP, dGTP, dTTP) were supplied by Genomyx Corporation. The full length T7 22-mer (GTAATACGACTCACTATAGGGC; SEQ ID NO: 2) and M13R(−48) 24-mer (AGCGGATAACAATTTCACACAGGA: SEQ ID NO: 3) were supplied by either Genomyx Corporation or Keystone Laboratories. BioMax Film was from Kodak.

Reverse Transcription.

For each reverse transcription reaction, 50 ng of mRNA was incubated with a 3′ Anchored Primer (1 μM) at 65° C. for 5 minutes. The tubes were chilled and spun briefly. The following reagents (with the final concentrations in parentheses) were added: first strand buffer (1×), dNTP mix (25 μM each), DTT (10 mM), ribonuclease inhibitor (1 unit/μl), and Superscript II Reverse Transcriptase (2 units/μl). The final volume was 20 μl. Tubes were heated to 25° C. for 10 min, 42° C. for 60 min, and 70° C. for 15 min. The cDNA produced was either used immediately or stored at −20° C.

Differential Display PCR.

Each DD-PCR was performed in duplicate, and contained the following reagents: PCR buffer II (1×), dNTP mix (20 μM each), a 5′ arbitrary primer (0.2 μM), the appropriate anchored primer (0.2 μM), Redivue [α-³³P]dATP (0.125 μCi/μl), AmpliTaq DNA Polymerase (0.05 units/μl), 2 μl of the reverse transcription reaction (above) and water to a final volume of 20 μl. The DD-PCR was performed under the conditions recommended by Genomyx Corporation: 95° C. for 2 min; 4 cycles of 92° C. for 15 sec, 46° C. for 30 sec, 72° C. for 2 min; 25 cycles of 92° C. for 15 sec, 60° C. for 30 sec, 72° C. for 2 min; and one cycle of 72° C. for 7 min, followed by cooling at 4° C.

Electrophoresis and Band Reamplification.

Stop solution (11 μl) was added to each reaction. The tubes were then heated for 2 min at 95° C. A 3-μl aliquot of each reaction was run on a 4.5% denaturing polyacrylamide gel for 16 hours at 800 V, 50° C. Under these conditions, bands ranging from 300 to 1200 base-pairs were well-resolved. Band excision and reamplification were performed according to the instructions given in the Genomyx Corporation protocol. The reamplification reaction mixture was added directly to the excised band and the PCRs were performed under the same conditions as the original DD-PCR, with the exceptions that the M13R(−48) and T7 primers (SEQ ID NO: 3 AND SEQ ID NO: 2, respectively) were used instead of the original anchored and arbitrary primers and [α-³³P]dATP was omitted. The PCR products were purified with S-400 HR microspin columns (Pharmacia).

PCR Product Subcloning.

PCR products were sequenced by cycle sequencing (see e.g., Beuss et al., Nucleic Acids Research 25:2233-2235 (1997); McMahon et al., Proc. Natl. Acad. Sci. USA 84:4974-4978 (1987)) using the M13R(−48) 24-mer primer (SEQ ID NO: 3). Generally, over 300 bases of sequence were obtained and used to search the non-redundant Genbank and dbEST databases using the BLASTN program (see e.g. Altschul et al., Nucleic Acids Res. 25:3389-3402 (1997)). Most of the PCR products were subcloned into the pT7Blue-1, pSTBlue-1 or pBSSK vectors using the T-A Cloning or the Perfectly Blunt Cloning Kits available from Novagen (Madison, Wis., USA). The plasmids were sequenced using the U-19 (GTTTTCCCAGTCACGACGT; SEQ ID NO: 4) and/or R-20 (CAGCTATGACCATGATTACG; SEQ ID NO: 5) sequencing primers (Novagen). Plasmid sequences were verified by alignment to the original PCR product sequence using the BLAST 2 Sequences program (see e.g., Tatusova and Madden, FEMS Microbiol. Lett. 174:247-250 (1999)). The plasmid sequences have been submitted to Genbank (http://www.ncbi.nlm.nih.gov/) with the following accession numbers: A24-1 (AF202328), A94-3 (AF202329), A94-4 (AF202330), A95-1 (AF202331), A96-4 (AF202332), A99-1 (AF202333), A102-1, 3′ end (AF202334), A102-1, 5′ end (AF202335), A104-5, 3′ end (AF202336), A104-5, 5′ end (AF202337), A105-7, 5′ end (AF202338), A105-7, 3′ end (AF202339), Al 11-8 (AF202340), Al 15-5 (AF202341), A124-1 (AF202342), A124-6 (AF202343), A128-7, 3′ end (AF202344), A128-7, 5′ end (AF202345), A130-3 (AF202346), A131-1 (AF202347), A135-3 (AF202348), A136-1 (AF202349), A155-6, 3′ end (AF202350), A155-6, 5′ end (AF202351), A160-5 (AF202352), A176-3, 3′ end (AF202353), A176-3, 5′ end (AF202354), A182-1 (AF202355), A183-1, 3′ end (AF202356) A183-1, 5′ end (AF202357), A187-5 (AF202358), 20-2, 3′ end (AF202359), 20-2, 5′ end (AF202360), 21-1, 3′ end (AF202361), 27-2, 3′ end (AF202362), 30-5, 5′ end (AF202363), 30-5, 3′ end (AF202364), 31-4, 5′ end (AF202365), 31-4, 3′ end (AF202366), 32-2, 3′ end (AF202367), 65-1, 5′ end (AF202368), 65-1, 3′ end (AF202369), 81-6, 3′ end (AF202370), 81-6, 5′ end (AF202371), 102-2 (AF202372), 103-2 (AF202373).

In addition, some clones were obtained by matching the PCR product sequences to the GenBank EST database (see e.g., Boguski and Schuler, Nature Genetics 10:369-371 (1995); Adams et al., Science 252:1651-1656 (1991)) and ordering the IMAGE Consortium clones (see e.g., Lennon et al, Genomics 33:151-152 (1996)) from commercial distributors. IMAGE clones obtained in this manner include the following (with the corresponding DD-PCR clones in parentheses): 223002 (A108D), 124345 (A136), 236199 (A185), 283163 (A123), 359102 (A172), 609386 (93), 1637906 (24), 269123 (101), 713625 (90-1), 1341231 (83), 845677 (23), 1629587 (74), 841495 (84), 320888 (87), 758242 (98), and 144992 (82). These clones were also sequenced and compared with the original PCR product.

D. Dot blot array

Dot Blot Preparation.

Single colonies were chosen for colony PCR, using the R-20 (SEQ ID NO: 5) and U-19 (SEQ ID NO: 4) primers. The quality of the PCR reactions was assessed by agarose gel electrophoresis. Human genomic DNA (Clontech) and PCR products were robotically dotted in 100 nl aliquots onto positively-charged nylon membranes using the BioDot instrument (Cartesian Technologies, Inc.). After uv-crosslinking, the membranes were rinsed in 2× SSC and allowed to air-dry. Prior to addition of labeled cDNA probes, membranes were washed in boiling 1% SDS, rinsed with 6× SSC, and incubated in 5 mL of 42° C. Microhyb solution (Research Genetics) for 2 hr. Ten minutes prior to addition of the probes, the Microhyb solution was replaced with an equal amount of fresh 42° C. Microhyb solution containing denatured human Cot-1 DNA (Gibco BRL) and poly(dA) primer (Research Genetics) (both at final concentrations of 1 ng/μl).

Probe Synthesis, Hybridization and Scanning of Filters.

For each reverse transcription reaction, 2 μg of mRNA was incubated with oligo(dT) primer (200 ng/μl) at 70° C. for 10 minutes. Tubes were chilled and spun briefly. The following reagents (with the final concentrations in parentheses) were added: first strand buffer (1×), DTT (10 mM), dNTP mix (1 mM each of dATP, dGTP, dTTP), [α-³³P]dCTP (3.3 μCi/μl) and Superscript II Reverse Transcriptase (10 units/μL). The samples were kept at 37° C. for 1.5 hr. Unincorporated nucleotides were removed by spinning the reaction mixture through a G-50 column. Incorporation rates ranged from 45 to 75%. Probe quality was assessed by electrophoresis on a 10% denaturing polyacrylamide minigel. Denatured probes were added directly to the Microhyb solution and hybridized overnight at 42° C. Membranes were washed twice under each of the following conditions: (1) 2× SSC/0.1% SDS at room temperature, 5 min; (2) 0.2× SSC/0.1% SDS at room temperature, 5 min; (3) 0.2× SSC/0.1% SDS at 42° C., 15 min, (4) 0.1× SSC/0.1% SDS at 68° C., 15 min. Membranes were then rinsed briefly in 2× SSC at room temperature, covered with Saran wrap, and exposed to storage phoshpor screens. After three days, screens were scanned using a Storm phosphorimager (Molecular Dynamics). Images were analyzed using ImageQuant software (Molecular Dynamics).

E. In situ Hybridization Assays

Probe Preparation.

Plasmids were linearized by restriction digestion and treated with proteinase K for 30 min at 50° C. Probe templates were then extracted twice with phenol-chloroform-isoamyl alcohol, EtOH-precipitated, washed, and resuspended in DEPC-treated water. Labeled antisense riboprobes were then prepared using the Ambion Maxiscript T7 or T3 transcription kits and [³³P]UTP (Amersham). Unincorporated nucleotides were removed by spinning the reaction mixture through a G-50 column (Pharmacia). [α-³³P] UTP incorporation rates typically ranged from 30 to 70%. Probe quality was assessed by electrophoresis on 6 or 10% denaturing polyacrylamide minigels.

Hybridization.

HepG2 cells were plated as described above in Amersham 96-well Cytostar T-plates. After treatment, media was aspirated from the wells. The cells were fixed with 100 μl/well of 4% formaldehyde in PBS for 10 min and then permeabilized with 100 μl of 0.25% Triton X-100 in PBS (warmed to 37° C.) for 1 hr. The 20 μl of labeled riboprobe solution was mixed with 800-900 μl of 10% (w/v) dextran sulfate, 50% formamide, 0.3 M NaCl, 10 mM Tris, pH 8.0, 1 mM EDTA, 10 mM DTT, and 0.5 mg/mL yeast tRNA in 1× Denhardt's solution. 50 μl of this solution was added to each well. Plates were sealed and incubated overnight at 50° C. On the following day, each well was washed three times with 1× SSC (250 μl per well). Excess probe was digested, with gentle shaking, for 30 min with 100 μl of 20 μg/ml RNase A in a buffer consisting of 10 mM Tris, pH 8.0, 0.5 M NaCl and 1 mM EDTA. After RNase A treatment, each well was shaken with 250 μl of the same buffer without RNase for 10 min. Wells were washed twice with 250 μl 0.25× SSC for a total of 45 min at 65° C. Plates were counted on a Packard TopCount instrument.

II. Results

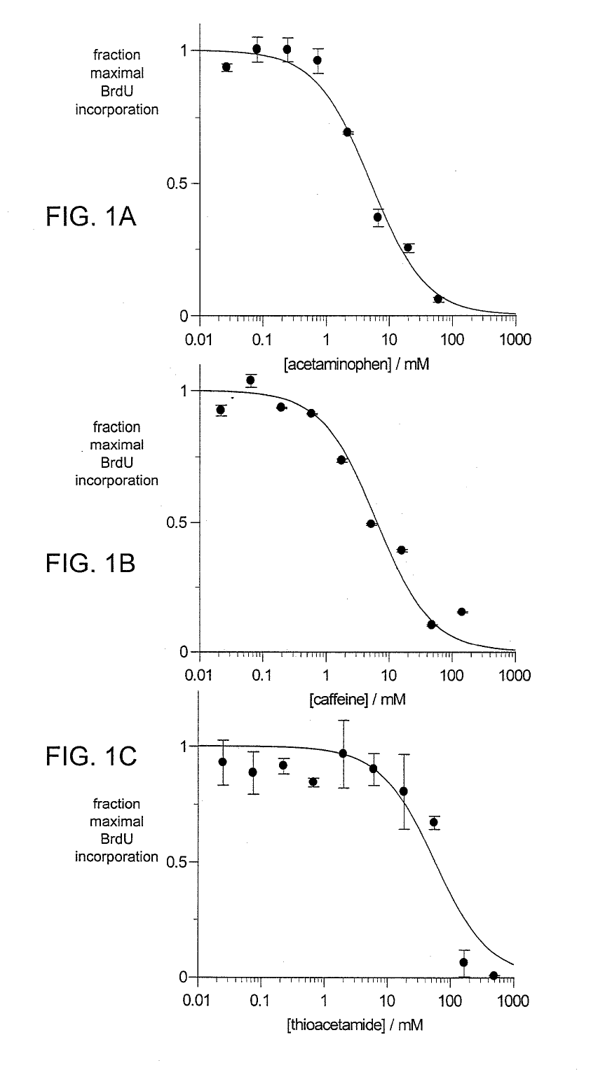

The general strategy used for identifying toxicant-induced gene expression changes is outlined in Table 2. In a preliminary DD-PCR experiment, very few gene expression changes were observed in samples from cells treated with doses of acetaminophen below the IC₅₀ for cell proliferation (Table 3; FIG. 1A). However, at very high doses, a loss of mRNA in a plate-based oligo(dT) hybridization assay was observed; this loss may have been brought about by a general down-regulation of transcription, by degradation of RNA, or by lift-off of cells from the plate surface. In order to maximize observable expression changes, we sought treatment conditions for subsequent DD-PCR experiments that gave significant inhibition of cell proliferation with no decrease in overall mRNA concentration. These criteria were met by 24-hour exposures to 20 mM acetaminophen, 16 mM caffeine, or 100 mM thioacetamide. Under these conditions, BrdU uptake was inhibited by 67 to 80% (FIGS. 1A-C) and cell morphology was visibly affected. The acetaminophen-treated cells appeared elongated and somewhat sparse, the caffeine-treated cells were generally rounded and slightly less adherent, and the thioacetamide-treated cells appeared somewhat dense and grainy.

For each treatment, the mRNA yields were comparable for treated and control samples, generally in the range of 25 to 40 μg of RNA from approximately 3×10⁷ cells. DD-PCR on samples from HepG2 cells at different passage numbers (15 and 36) gave identical banding patterns (data not shown); nonetheless, cultures were generally discarded after 6 months (70 passages). RNA sample quality, as assessed by agarose gel electrophoresis and by the appearance of the DD gels, was also comparable between treated and control samples. The use of mRNA rather than the more customary total RNA was supported by two observations. First, comparison of DD-PCR bands from mRNA and total RNA resulted in only one major band that was unique to the total RNA lanes. DNA sequence analysis of this band indicated strong homology to 16S ribosomal RNA. Second, agarose gel electrophoresis and control DD-PCR reactions performed without reverse transcriptase indicated no significant genomic DNA contamination.

As shown in Table 4, the mRNA samples were subjected to DD-PCR using three different sets of primer pairs. Differentially displayed bands in the range of 350 to 1200 bp that arose in duplicate DD-PCR reactions were excised from the gels and PCR-amplified using the M13R(−48) (SEQ ID NO: 3) and T7 (SEQ ID NO: 2) primers. Of 173 bands excised, 139 yielded PCR products of the correct size, and in sufficient quantity for further analysis (Table 5). These PCR products were purified through G-50 spin columns and cycle-sequenced using the M13R(−48) 5′ universal primer (SEQ ID NO: 3). In other experiments, we found that the T7 3′ primer (SEQ ID NO: 2) gave low-quality sequence, probably because of variations in the length of the poly(A) sequence; such variability was observed in subclones (data not shown). Of the 139 PCR products, 110 gave readable sequences, indicating the predominance of one species after reamplification. Generally, over 300 bp of sequence was obtained and used in BLASTN searches of the dbEST and non-redundant GenBank databases (see e.g., Altschul et al., Nucleic Acids Res. 25:3389-3402 (1997)). The best human gene matches are listed in Table 6. The 110 bands that gave readable sequence represented only 79 unique sequences. Of these, 31 of the PCR products were subcloned, and an additional 15 were obtained as IMAGE clones from commercial sources (see e.g., Lennon et al., Genomics 33:151-152 (1996)). In the process, four subclones and one IMAGE clone that did not match the original PCR sequences were obtained.

We employed a rapid dot blot assay as a secondary screen for gene expression changes. We tested each of the unique clones against each of the three treatments. We included the five clones whose sequences did not match the PCR products. For these clones, the dot blot assay functioned not as a confirmation assay but as an initial screen for differential expression. Dot blots were prepared by robotically arraying subclone-derived PCR products in quadruplicate onto positively charged nylon membranes. We found that robotically dotted arrays gave more reproducible results than manually produced blots. In general, each dot consisted of over 80 ng of PCR product, as estimated by inspection of the PCR reactions run on agarose gels. This high quantity of DNA ensured that saturation of spots, with consequent loss of quantitation, would not occur. Spots of genomic DNA were included on each filter to allow normalization between control and treated sample intensities.

When hybridized with [³³P]cDNA derived from the mRNA samples, the 51 clones listed in Table 6 gave measurable spot intensities; nine genes did not give measurable intensity in any sample. Using a two-fold change in spot intensity as a threshold for differential expression, over half (26 of 48) of the DD-PCR observations were confirmed by this assay. Comparable confirmation rates were observed among the three treatments. Of the 51 genes examined, 38 showed at least a two-fold change in response to one or more of the treatments; 72% of these changes were down-regulations. Nine genes showed a similar change with all three compounds Table 7.

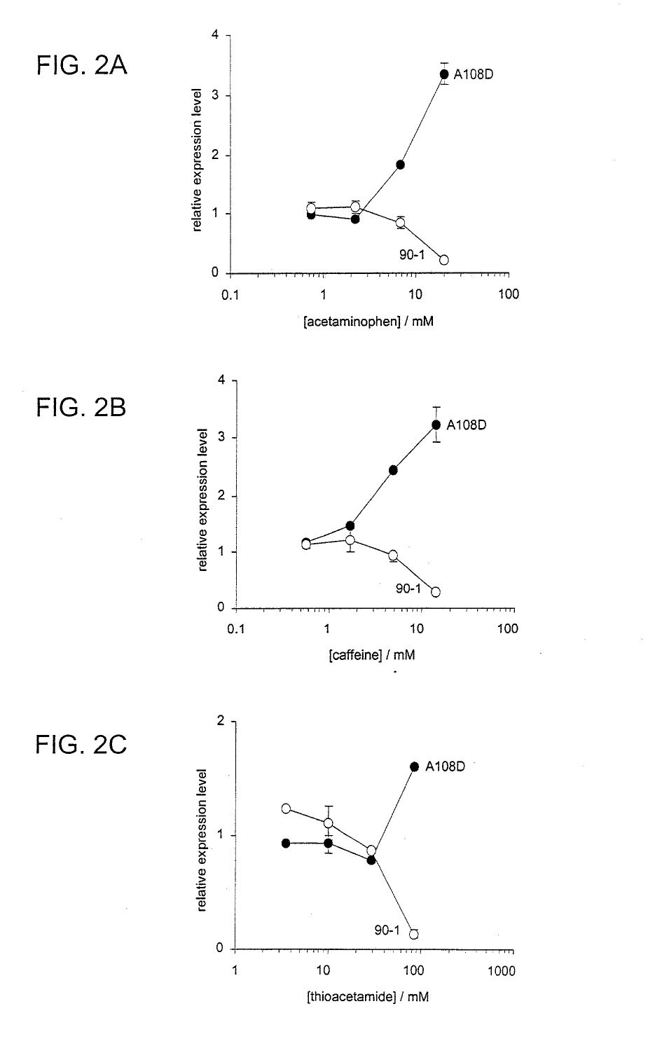

Selected clones were also tested in a 96-well plate in situ hybridization assay using ³³P-labeled riboprobes prepared by in vitro transcription from subclone-derived templates (see e.g., Harris et al., Anal. Biochem. 243:249-256 (1996)). This assay provides a convenient format for dose-response curves without the need for preparing RNA. Results from the plate assay are generally in agreement with results from the dot blot assay or Northern blots (data not shown). Several representative dose-response curves are shown in FIGS. 2A-C. We tested 16 clones in this assay against all three compounds, and in no case did we observe a two-fold gene expression change at a non-toxic dose; in most cases a dose above the IC₅₀ was required.

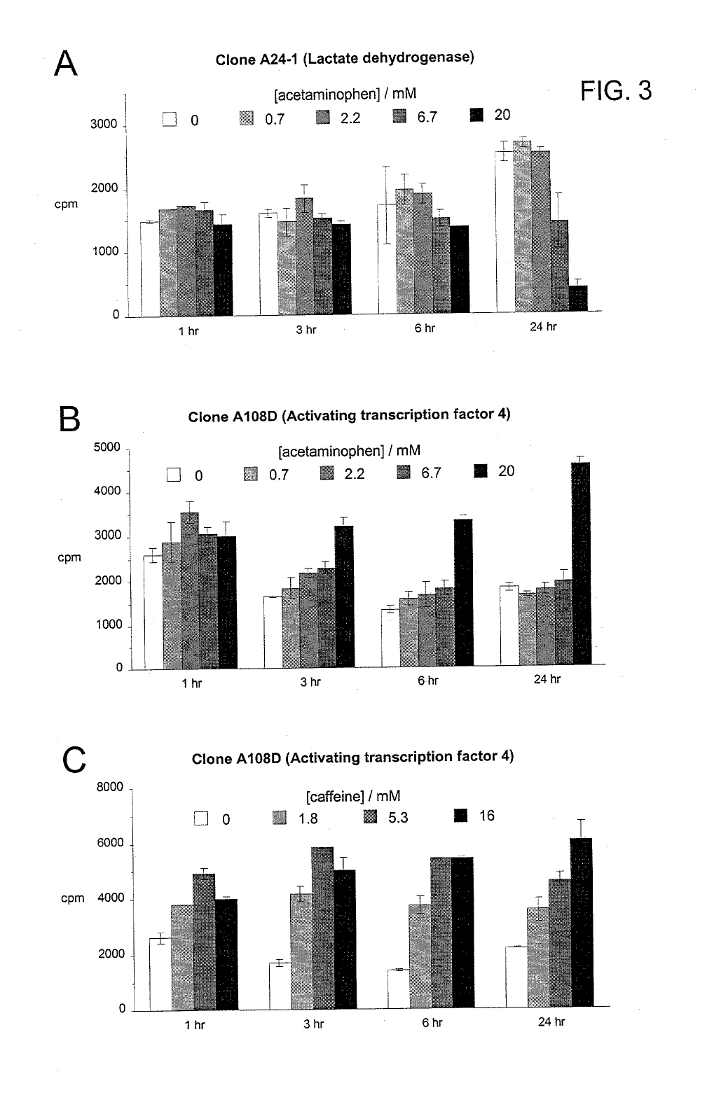

We also used the plate assay to examine expression changes over time and dose for several clones (FIGS. 3A-C). Relative to controls, activating transcription factor 4 (ATF-4) transcript levels increased with time and concentration of caffeine. However, in acetaminophen-treated cells, only the highest concentration elicited an increase in ATF-4 transcripts. Decrease in lactate dehydrogenase gene transcription was observed only at the 24-hour timepoint.

III. Discussion

Unlike high-density microarrays (see e.g., Schena et al., Science 270:467-470 (1995); Lockhart et al., Nature Biotechnology 14:1675-1680 (1996); Dugan et al., Nature Genetics supplement 21:10-14 (1999)), DD-PCR is an open system for discovering differentially expressed genes. No prior knowledge of gene sequences is required, and the PCR conditions are of such low stringency that only the 5-6 bases at the 3′ end of each primer need match a potential PCR template (see e.g., Liang and Pardee, Science 257:967-971 (1992)). Therefore, using appropriate primers one can detect most expressed genes. Furthermore, the starting materials and equipment are common in most molecular biology laboratories.

We incorporated a number of improvements to the original DD PCR technology to increase the overall efficiency of the process (see e.g., Martin and Pardee, Methods Enzymol 303:234-258 (1999); and Linskens et al., Nucleic Acids Research 23:3244-3251 (1995), both of which are incorporated herein by reference in their entirety). For example, we ran duplicate reactions on high-resolution acrylamide gels, and only excised bands greater than 350 bases long. Care was also taken to accurately isolate and identify the differentially displayed bands. In this regard, we found cycle sequencing of the reamplified PCR products to be an extremely useful practice for several reasons. First, this approach allowed us to eliminate heterogeneous bands at an early stage because they produce mixed, unreadable sequences. Second, comparisons of PCR product sequences within an experiment allowed us to minimize the subcloning of redundant species. For example, in 12 cases, two bands that migrated close to each other were each excised and reamplified, and upon sequencing found to be homologous. Presumably, these pairs represent complementary strands of the same PCR products. Redundancy also arose from related sequences being amplified by different primer pairs in the DD-PCR reactions. For example, the lactate dehydrogenase-A gene was represented by three individual bands, two from acetaminophen samples and one from thioacetamide. Although such redundancy within or across experiments can be problematic, we did observe that the more frequently a sequence appeared, the more likely was confirmation in a secondary assay.

A third advantage of cycle sequencing was a reduced need for in-house subcloning as a source of clones for confirmation assays. In many cases, homologous clones from the IMAGE collection were ordered from commercial sources. However, we found that because of errors or contamination in the commercial stocks, these clones had to be restreaked and sequence-verified. Occasionally, we obtained IMAGE clones or PCR product subclones that did not match the sequence of the amplified gel band. We tested these clones anyway (Table 6).

We adopted a “matrix” approach to our DD-PCR experiments. Messenger RNA samples from three different treatments were each subjected to partial DD-PCR analysis, using three non-overlapping sets of primer pairs. Subclones obtained from these experiments were then subjected to a rapid secondary assay to: (1) confirm differential expression in the original treatment and (2) test for differential expression in the other two treatments. The three toxicants, acetaminophen, caffeine, and thioacetamide, were chosen because they show measurable cytotoxicity in HepG2 cells in our assays. These compounds are likely to operate through a number of toxic mechanisms, including mitochondrial disruption, perturbation of calcium homeostasis, macromolecular binding, genotoxicity and lipid peroxidation (see e.g., Möller and Dargel, Acta pharmacol. et toxicol. 55: 126-132 (1984); Burcham and Harman, Toxicology Letters 50:37-48 (1990); Burcham and Harman, J. Biol. Chem. 266:5049-5054 (1991); D'Ambrosio, Regulatory toxicology and pharmacology 19:243-281 (1994); and Casarett and Doull 's Toxicology: The Basic Science of Poisons, (Klaasen, C. D., Ed.), McGraw-Hill, New York, (1996)).

For DD-PCR analysis, we used a total of 42 primer pairs, giving us genome coverage of about 20% across the three treatments. This level of coverage compares favorably with most current array-based expression monitoring approaches, which typically sample 4,000-10,000 genes, or less than 10% of the genome (see e.g., Duggan et al., Nature Genetics supplement 21:10-14 (1999)). The strategy of combining a “matrix” DD-PCR strategy with a rapid secondary assay enabled us to find nine genes whose confirmed expression changes were similar for all three of the 24-hour treatments (Table 7).

In addition to these nine genes, we discovered a number of other genes that were affected by one or two of the treatments. In all, we observed 38 genes or ESTs whose expression was modulated by at least two-fold in one or more treatments. Roughly one-third of these modulated sequences are ESTs. The remaining sequences include a large proportion of genes encoding enzymes involved in cellular metabolism, such as lactate dehydrogenase-A, pyruvate dehydrogenase and NADH dehydrogenase. In most cases, these “housekeeping” genes were down-regulated. Genes for some proteins possibly involved in cellular stress responses were observed to be up-regulated, including heat shock protein 90, the cAMP-dependent transcription factor ATF-4, and an EST similar to ubiquitin hydrolase (GenBank AI131502). ATF-4 showed the largest consistent up-regulation, with a 3.8- to 10.5-fold increase in expression across the three treatments.

Overall, almost three-fourths of the expression changes were found to be down-regulations, which may indicate a general shutdown of many cellular functions by the time the cells have been exposed to a fairly high dose of toxicant for 24 hr. In separate experiments using cDNA arrays (see Example 2), we observed a greater number of expression changes at earlier time points, including a higher proportion of up-regulations.

Twenty-seven clones fell into one of two categories: they either failed to confirm with the original treatment or they did not match the sequences of the PCR products derived from the excised bands. Some of these genes may in fact be modulated to some extent by the treatment in question, but nevertheless failed to show an effect in the secondary assay. However, for the sake of argument, they can be considered randomly isolated clones. Of these 27 clones, 7 show an expression change in response to acetaminophen, 7 in response to caffeine, and 9 in response to thioacetamide (Table 6). Thus, the hit rate for any one compound was as high as 33% with this set of clones. These results indicate that even a strategy based on randomly picking clones would have yielded many genes of interest. For treatment conditions eliciting fewer gene expression changes, this sort of random approach would no doubt be less effective.

In situ hybridization assays in 96-well plates allowed a more detailed study on a subset of the clones at a variety of doses and time points, and revealed certain nuances in expression (FIGS. 2A-C and 3A-C). ATF-4, an up-regulated gene, showed an early response in both acetaminophen and caffeine; while LDH-A, a down-regulated gene, did not drop until after the 6-hour timepoint. In addition, the dose-response profiles for ATF-4 differed markedly between acetaminophen and caffeine. These observations indicate that a variety of expression profiles can be observed over the course of cellular response to toxic injury, and are supported by results using array-based expression monitoring methods (see Example 2). These results also indicate that studying expression at a single time point may limit the transcriptional changes observed to a subset of the affected genes.

The results indicate that the expression changes observed are coincident with the toxic effects of the toxicants and not simply incidental effects that reflect the progression of the cell toward growth arrest and death. First, DD-PCR performed at low doses of acetaminophen, below the concentration required to cause a measurable inhibition of cell proliferation, yielded very few expression changes (Table 3). Second, dose-response curves for expression of several individual genes showed that substantial expression changes (greater than two-fold) did not occur at non-toxic concentrations (FIGS. 2A-C and 3A-C).

TABLE 2
Experimental strategy

| Step | Comments |
|---|---|
| 1. Treatment of cells | Doses of acetaminophen, caffeine and thioacetamide were chosen to give |
| | significant inhibition of cell proliferation in a BrdU incorporation assay |
| 2. Preparation of mRNA | mRNA was affinity purified on oligo(dT) cellulose and examined for |
| | degradation by agarose gel electrophoresis |
| 3. DD-PCR | Reactions were performed using different sets of primer pairs for each |
| | treatment in order to maximize genome coverage |
| 4. Isolation of differentially | Bands of interest were excised and PCR-amplified |
| displayed bands | |
| 5. Sequencing of amplified | PCR products were cycle-sequenced; those giving poor, mixed or |
| bands | redundant sequences were eliminated |
| 6. Database search | Matches to sequences in public databases were identified by BLAST |
| | searches |
| 7. Acquisition of clones | Clones of sequences of interest were obtained either by subcloning the |
| | PCR products or purchasing the corresponding IMAGE clones |
| 8. Secondary assays | Differential expression of clones of interest was tested in dot blot assays, |
| | with further characterization in plate-based in situ hybridization assays |

TABLE 3
Effect of acetaminophen dose on the number of expression
changes observed by DD-PCR

Number of difference bands on DD-PCR gel¹

| Dose, mM | Increased | Decreased |
|---|---|---|
| 0.02 | 0 | 0 |
| 0.2 | 0 | 0 |
| 2 | 4 | 1 |
| 20 | 18 | 16 |

¹Difference bands were identified by visual inspection of DD-PCR gels and do not reflect confirmed expression changes.

TABLE 4
Primer pairs used in DD-PCR reactions¹

| | Anchored primer³ | AP2- | | | | | |
|---|---|---|---|---|---|---|---|
| Arbitrary primer² | SEQ ID NO: | GC | AP3-GG | AP4-GT | AP5-CA | AP8-AA | AP9-AC |
| ARP1 CGACTCCAAG | SEQ ID NO: 6 | THI | APAP | APAP | APAP | THI | |
| ARP2 GCTAGCATGG | SEQ ID NO: 7 | THI | | | | THI | |
| ARP3 GACCATTGCA | SEQ ID NO: 8 | THI | APAP | APAP | APAP | THI | |
| ARP4 GCTAGCAGAC | SEQ ID NO: 9 | THI | | | | THI | |
| ARP5 ATGGTCGTCT | SEQ ID NO: 10 | | APAP | APAP | APAP | | |
| ARP6 TACAACGAGG | SEQ ID NO: 11 | | APAP | APAP | APAP | | |
| ARP7 TGGATTGGTC | SEQ ID NO: 12 | | APAP | | APAP | | |
| ARP8 TGGTAAAGGG | SEQ ID NO: 13 | CAF | | | | CAF | |
| ARP9 TAAGCCTAGC | SEQ ID NO: 14 | CAF | | | | CAF | |
| ARP10 GATCTCAGAC | SEQ ID NO: 15 | CAF | | | | CAF | |
| ARP11 ACGCTAGTGT | SEQ ID NO: 16 | CAF | | | | CAF | |
| ARP12 GGTACTAAGG | SEQ ID NO: 17 | CAF | | | | CAF | |
| ARP14 TCCATGACTC | SEQ ID NO: 18 | THI | | | | | THI |
| ARP17 CTGCTAGGTA | SEQ ID NO: 19 | THI | | | | | THI |
| ARP18 TGATGCTACC | SEQ ID NO: 20 | THI | | | | | THI |
| ARP19 TTTTGGCTCC | SEQ ID NO: 21 | THI | | | | | THI |
| ARP20 TCGATACAGG | SEQ ID NO: 22 | THI | | | | | THI |

¹DD-PCR reactions were performed using mRNA samples derived from cells treated with acetaminophen (APAP), caffeine (CAF) or thioacetamide (THI).
²Each 5′ arbitrary primer (ARP) consists of the M13R(−48) primer sequence (ACAATTTCACACAGGA) (SEQ ID NO:3)
³Each anchored primer (AP) consists of the T7 RNA polymerase sequence (ACGACTCACTATAGGGC) (SEQ ID NO:2) followed by T₁₂ and the two “anchoring” nucleotides shown at the 3′ end.

TABLE 5
Numbers of clones passing successive stages of differential display experiments

| | Acetaminophen | Caffeine | Thioacetamide |
|---|---|---|---|
| DD GEL BANDS ISOLATED | 39 | 80 | 54 |
| Gel bands successfully amplified | 33 | 59 | 47 |
| Readable sequences from amplified bands | 24 | 48 | 38 |
| Unique sequences¹ | 21 | 32 | 26 |
| Unique clones quantitated on dot blot arrays² | 9 | 20 | 26 |

¹Unique sequences within a treatment; redundancy across treatments is not reflected in these numbers.
²Several clones gave undetectable signal on dot blot arrays are are not included in these numbers. Due to redundancy across treatments, the overall number of clones tested was only 51 (see TABLE 6).

TABLE 6
Custom array measurements of effects of three compounds on expression of genes identified in DD-PCR experiments

| DD-PCR | | Direction of | | Dot blot expression ratio | | |
|---|---|---|---|---|---|---|
| clone | Initial | DD-PCR | | (treated/control)⁵ | | |
| number¹ | treatment² | change | BLAST result (best human gene match)⁴ | APAP | CAF | THI |
| A102-1 | APAP, THI | up | EST (AA581887) | 2.22 c | 3.45 | 1.78 n |
| A94-3 | APAP, THI | down | Lipoprotein-associated coagulation inhibitor | 0.21 c | 0.58 | 0.27 c |
| A24-1 | APAP, THI | down | Lactate dehydrogenase A | 0.11 c | 0.25 | 0.20 c |
| A105-7 | APAP | down | EST, similar to Long-chain acyl-coenzyme A synthetase | 0.00 c | 3.77 | 0.90 |
| A95-1 | APAP | no change | EST (AC007400) | 0.82 c | 2.83 | 1.07 |
| A96-4 | APAP | down | ALU WARNING: Human Alu-Sc subfamily consensus sequence | 0.70 n | 0.78 | 0.84 |
| A99-1 | APAP | down | EST (N39662) | 0.76 n | 1.30 | 0.50 |
| A104-5 | APAP | up | EST (AI049999) | 1.09 n | 0.72 | 0.89 |
| A94-4 | APAP³ | | Cu/Zn superoxide dismutase (SOD) | 1.22 | 0.77 | 0.91 |
| A108D | CAF | up | Activating transcription factor 4 | 8.81 | 10.48 c | 3.77 |
| A131-1 | CAF | up | NADH dehydrogenase subunit 2 | 0.92 | 5.40 c | 1.45 |
| A136 | CAF | up | Centromere protein F (400 kD) (CENPF kinetochore protein) | 1.36 | 2.31 c | 1.98 |
| A135-3 | CAF | down | Human transposon-like element mRNA | 1.12 | 0.40 c | 0.59 |
| A124-1 | CAF, THI | down | Apolipoprotein B-100 | 0.71 | 0.34 c | 0.76 |
| A185 | CAF | down | procollagen-lysine 2-oxoglutarate 5-dioxygenase 2 | 0.65 | 0.34 c | 0.36 |
| A160-5 | CAF | down | EST (AA430551) | 1.66 | 0.27 c | 0.17 |
| A115-5 | CAF | down | Lsm5 protein | 1.12 | 0.26 c | 0.39 |
| A123 | CAF | down | pyruvate dehydrogenase El-beta subunit | 0.32 | 0.20 c | 0.08 |
| A155-6 | CAF | down | Transforming growth factor-beta type III receptor | 0.47 | 0.12 c | 0.33 |
| A130-3 | CAF | up | EST, similar to ubiquitin hydrolase | up | up c | up |
| A136-1 | CAF | up | AH antigen | 1.89 | 1.80 n | 0.00 |
| A172 | CAF | down | DNA topoisomerase II binding protein | 0.33 | 1.03 n | 0.49 |
| A176-3 | CAF | down | DB1 | 0.75 | 1.48 n | 0.36 |
| A183-1 | CAF | up | EST, bithoraxoid-like protein | 1.44 | 0.90 n | 0.42 |
| A187-5 | CAF | up | Centromere protein E (CENPE) | 0.86 | 1.03 n | 0.65 |
| A182-1 | CAF | down | Atopy related autoantigen CALC | 1.62 | 0.63 n | 0.66 |
| A111-8 | CAF | down | High mobility group 2 protein (HMG-2) | 0.56 | 0.66 n | 1.12 |
| A124-6 | CAF | down | EST (N22016) | up | up n | up |
| A128-7 | CAF | up | Liver microsomal UDP-glucuronosyltransferase (UDPGT) | 0.79 | 1.25 n | 0.70 |
| 27-2 | THI | down | Ku autoimmune antigen | 1.22 | 1.37 | 0.47 c |
| 93 | THI | down | EST, similar to Ubiquinol cytochrome C reductase core protein 2 | 0.86 | 0.42 | 0.38 c |
| 24 | THI | down | Esterase D/formylglutathione hydrolase | 0.39 | 0.68 | 0.31 c |
| 101 | THI | down | EST (N26592) | 0.93 | 0.83 | 0.26 c |
| 81-6 | THI | down | E1B 19K/Bcl-2-binding protein Nip3 | 0.79 | 0.33 | 0.23 c |
| 30-5 | THI | down | PPP1R5 gene | 0.40 | 1.51 | 0.17 c |
| 90-1 | THI | down | EST (AA283846) | 0.29 | 0.15 | 0.13 c |
| 32-2 | THI | down | EST (AI310515) | 0.33 | 0.12 | 0.11 c |
| 83 | THI | down | EST (AA805555) | 0.28 | 0.19 | 0.09 c |
| 20-2 | THI | up | Nucleosome assembly protein 1-1ike 1 (NAP1L1) | 1.32 | 1.11 | 0.92 n |
| 23 | THI | up | 90-kDa heat-shock protein | 1.23 | 2.67 | 0.96 n |
| 65-1 | THI | up | Interleukin 6 signal transducer (gp130, oncostatin M receptor) | 1.51 | 0.93 | 0.96 n |
| 74 | THI | up | MEGF9 | 0.99 | 0.75 | 0.96 n |
| 84 | THI | down | EST, similar to arachidonate 15-lipoxygenase | 1.32 | 0.98 | 0.75 n |
| 87 | THI | up | EST (W44772) | 0.92 | 1.29 | 1.11 n |
| 98 | THI | down | cAMP-responsive enhancer binding protein, alt. spliced | 2.00 | 1.06 | 0.70 n |
| | | | (CREB327) | | | |
| 102-2 | THI | up | EST (AA581887) | 3.53 | 4.00 | 1.80 n |
| 103-2 | THI | up | G1 to S phasetransition 1 (GSPT1) | 2.17 | 2.57 | 1.57 n |
| 21-2 | THI³ | | T-complex polypeptide 1 | 0.39 | 0.45 | 1.03 |
| 23-1 | THI³ | | Glucose transporter pseudogene | 0.33 | 1.08 | 0.34 |
| 31-4 | THI³ | | ABC transporter | 0.54 | 0.13 | 0.28 |
| 82 | THI³ | | Myristoyl CoA:protein N-myristoyltransferase | 1.20 | 0.75 | 0.41 |
| ¹ Clone A99 shares sequence homology with clone 101; clone A102 shares sequence homology with clone 102. | | | | | | |
| ² Drug treatment in which expression change was initially observed by DD-PCR. APAP, acetaminophen; CAF, caffeine; THI, thioacetamide. | | | | | | |
| ³ The probe sequence did not match the sequence of the PCR product derived from the DD gel band, but nevertheless was tested in the dot blot assay. | | | | | | |
| ⁴ For ESTs with no homology to known genes, the accession number of the best BLAST match is indicated. | | | | | | |
| ⁵ Expression ratios are based on quadruplicate spots on dot blot arrays. Standard deviations were generally less than 25% of mean values. “Up” indicates measurable intensity in treated but not in control spots. A “c” indicates confirmation of the DD-PCR result, based on a change in spot intensity of at least two-fold; “n” indicates no confirmation. Several genes gave spot intensities too low to quantitiate with both control and treated samples and are | | | | | | |
| # not listed in this Table. | | | | | | |

TABLE 7
Genes showing similar expression
changes with all three toxicants

| | |
|---|---|
| Gen Bank | |
| Accession | Fold change¹ |

| | | | | | |
|---|---|---|---|---|---|
| Clone | No. | Gene | APAP | CAF | THI |

A. UP-REGULATION

| | | | | | |
|---|---|---|---|---|---|
| AI24-6 | N22016 | EST | up | up | up |
| A130-3 | A1131502 | EST, similar to ubiquitin | up | up | up |
| | | hydrolase | | | |
| A108D | D90209 | Activating transcription | 8.8 | 10.5 | 3.8 |
| | | factor 4 | | | |

B. DOWN-REGULATION

| | | | | | |
|---|---|---|---|---|---|
| A24-1 | HDS914 | Lactate dehydrogenase A | 9.1 | 4.0 | 5.0 |
| A123 | AA521401 | Pyruvate dehydrogenase | 3.1 | 5.0 | 12.5 |
| | | E1-beta subunit | | | |
| A155-6 | L07594 | Transforming growth | 2.1 | 8.3 | 3.0 |
| | | factor-beta type III | | | |
| | | receptor | | | |
| 90-1 | AA283846 | EST | 3.4 | 6.7 | 7.7 |
| 32-2 | AI310515 | EST | 3.0 | 8.3 | 9.1 |
| 83 | AA805555 | EST | 3.6 | 5.3 | 11.1 |

¹Fold changes are derived from the data in Table 5. “Up” indicates that the fold change could not be determined because expression was not detectable in control samples.

### EXAMPLE 2

### Differential Gene Expression in Response to the Toxicants Acetaminophen, Caffeine and Thioacetamide as Determined by Probe Arrays and Quantitative RT-PCR

This set of experiments utilized cDNA array methods coupled with quantitative RT-PCR to study the temporal expression patterns of over 5,000 genes in the HepG2 human liver cell line in response to the same three model hepatotoxicants used in Example 1, namely acetaminophen, caffeine and thioacetamide. Thus, the experiments paralleled those in Example 1, but utilized different assay techniques. As in Example 1, these studies were undertaken in part to identify common patterns of gene expression changes in order to gain mechanistic information on the development of toxicity and to develop toxicity assays.

I. Materials and Methods

A. Cytotoxicity and Apoptosis Assays

Cytotoxicity Assays.

HepG2 cells (ATCC HB-8065) were cultured in DMEM/F12 medium (Gibco-BRL) with 10% fetal bovine serum, plated into 96-well tissue culture treated plates at 10⁵ cells/well, and grown for 3 days prior to treatment, which was carried out in serum-free medium with 0.25% DMSO added to improve compound solubility. Cell proliferation assays based on measurement of BrdU incorporation were performed according to the manufacturer's instructions (Boehringer Mannheim “Cell Proliferation ELISA Kit”).

Annexin V Assay for Apoptosis.

Translocation of phosphatidyl serine to the cell membrane was measured by affinity binding to annexin V using the Apotest Biotin kit from NeXins Research B.V. (The Netherlands). HepG2 cells were cultured as above and plated into Cytostar-T scintillating microplates (Amersham) at 10⁶ cells/well and grown for 3 days prior to treatment as above. Following treatment, 50 μl/well of 4 μg/ml annexin V-biotin in 2× Ca²⁺ binding buffer was added. Wells with no annexin V-biotin were included as background controls. Following incubation for 20 min at room temperature, 50 μl/well of 0.5 μCi [³⁵S] streptavidin (Amersham) in 2× Ca²⁺ binding buffer was added and incubated for 2 hrs at room temperature with gentle shaking. Plates were spun down at 1,100 rpm for 8 min and read on a Packard TopCount instrument (see e.g., Vermes et al., J. Immunol. Methods 185:81-93 (1995)).

Caspase-3 assay for apoptosis. Activation of caspase-3, an intracellular cysteine protease, was measured by cleavage of a caspase-specific peptide using the Caspase-3 Colorimetric Assay kit from R&D Systems. HepG2 cells were cultured and treated as above in T-75 tissue culture flasks. Following treatment, cells were scraped off and spun down. The assay was performed according to the kit instructions using 350 μl/flask of lysis buffer.

Oligo(dT) Assay.

Following cell treatment as described above, cells were fixed with 100 μl/well 4% formaldehyde in PBS for 10 min at room temperature and then permeabilized with 100 μl/well 0.25% Triton X-100 in PBS for 1 hr at room temperature. 50 μl/well of 20 μg/ml 5′-biotin-oligo(dT₁₅) (Keystone) in DIG Easy Hyb (Boehringer-Mannheim) was added and incubated 16-18 hr at room temperature. Wells were washed 4 times with 100 μl/well 2× SSC, and then 100 μl/well of 1 μg/ml horseradish peroxidase-conjugated streptavidin (Pierce) in 1× Blocking buffer (Ambion) was added and incubated 1 hr at room temperature. After washing twice with 100 μl/well 1× washing buffer (Ambion), 100 μl/well TMB substrate (KPL) was added and the absorbance at 650 nm was measured.

B. Probe Array Methods

Cell Treatment and Preparation of mRNA.

Cells were grown in DMEM/F12 medium with 10% fetal bovine serum in tissue culture flasks for 3 days following splitting, at which time they were at or near confluency. Cells were treated with 20 mM acetaminophen, 16 mM caffeine, or 100 mM thioacetamide in serum-free DMEM/F12 plus 0.25% DMSO for times ranging from 1 to 24 hr. For each treated sample, an untreated control flask was set up with the same medium. Following the treatment period, mRNA was isolated by affinity purification on oligo(dT) cellulose resin using the Poly(A) Pure mRNA isolation kit from Ambion. RNA quality was assessed by agarose gel electrophoresis, and yields were determined by absorbance at 260 nm.

Preparation of Complex Target Nucleic Acids.

Radiolabeled cDNA for array hybridizations were prepared as follows. To a solution of 2 μg of RNA in 8 μl DEPC-treated water was added 2 μl of 1 μg/μl oligo(dT) (10-20 mer mixture, Research Genetics). After incubation for 10 min at 70° C., the solution was chilled on ice for 2 min, and then added to 6 μl of 5× first strand buffer (250 mM Tris-HCl (pH 8.3), 375 mM KCl, 15 mM MgCl₂; Gibco-BRL), 1 μl of 0.1M DTT, 1.5 μl dNTP mix (20 mM each dATP, dGTP and dTTP), 10 μl of 10 mCi/ml [α-³³P]dCTP (1000 Ci/mmol, Amersham), and 1.5 μl of 200 U/μl reverse transcriptase (Superscript II, Gibco-BRL). Following a 90 min incubation at 37° C., cDNA targets were purified by passage through G-50 Sephadex spin columns (Pharmacia) or Bio-Spin 6 columns (BioRad).

Hybridization to Arrays.

GF200 cDNA arrays (Research Genetics) were washed in 0.5% boiling SDS for 5 min and prehybridized for 3 hrs at 42° C. in 5 ml MicroHyb solution (Research Genetics) containing 5 μl of 1 μg/ml poly(dA) (Research Genetics) and 5 μl of 1 μg/ml human Cot-I DNA (Gibco-BRL) that was denatured for 3 min at 100° C. prior to use. Labeled target nucleic acids, boiled for 3 min, were added directly, and hybridization was allowed to proceed for 16-18 hr at 42° C. in roller bottles in hybridization ovens. Arrays were washed twice in 2× SSC, 1% SDS at room temperature for 2 min, and then twice in 0.5× SSC, 1% SDS at 65° C. for 20 min. Arrays were exposed to storage phosphor screens for 3 days and scanned using a phosphorimager (Molecular Dynamics). Arrays were stripped for reuse by placing in boiling 0.5% SDS and then incubating for 1.5 hr with shaking at room temperature, allowing to solution to cool. After stripping, arrays were exposed to storage phosphor screens overnight to confirm loss of signal.

Analysis of Array Data.

Spot intensities were determined using Pathways software (Research Genetics). Data from quadruplicate sets of hybridizations were normalized by local regression using NLR software (Tom Kepler, North Carolina State University). Cluster analysis was carried out using the Clustan Graphics software package from Clustan Limited (Edinburgh).

C. Confirmation Assays

Quantitative RT-PCR.

Primers and probes were designed using Primer Express software (Perkin-Elmer). TaqMan probes (Perkin-Elmer) were synthesized with reporter dye 6FAM at the 5′ end and quencher TAMRA at the 3′ end. RNA template concentrations were determined by absorbance at 260 nm. Reactions were performed as described (ref), using 2.5 ng RNA, 300 nM each PCR primer, and 150 nM Taqman probes. Control reactions were set up with reverse transcriptase or template omitted. Reactions were run on an ABI 7700 instrument (Perkin-Elmer) using the following cycling conditions: reverse transcription at 48° C. for 30 min; inactivation of reverse transcriptase at 95° C. for 10 min; 40 cycles of denaturation at 94° C. for 15 sec and extension at 60° C. for 1 min. Changes in expression were calculated from the displacement of the amplification curve in the treated sample relative to the control.

II. Results and Discussion

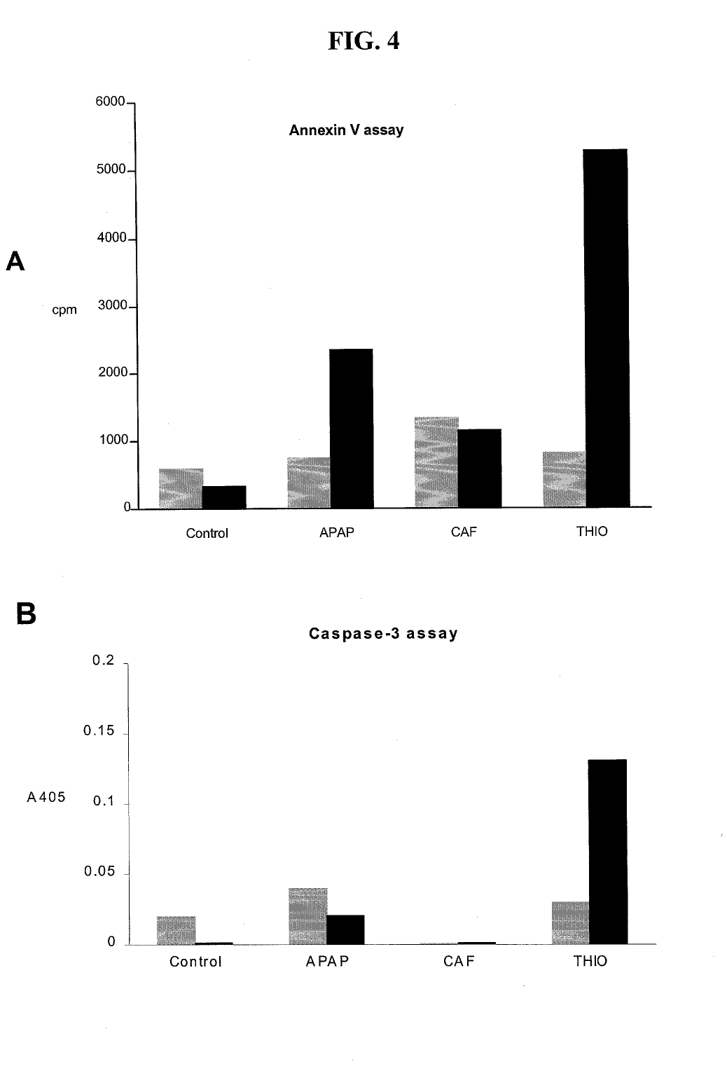

Our strategy for identifying cytotoxicity-associated gene expression changes is outlined in Table 8. For these experiments, we used doses of three compounds (20 mM acetaminophen, 16 mM caffeine, and 100 mM thioacetamide) that was shown in the set of experiments described in Example 1 to cause significant inhibition (67-80%) of HepG2 cell proliferation after 24 hr. Lower concentrations are not feasible for expression profiling studies, since at subtoxic doses very few gene expression changes are observed (see results from Example I). At higher doses, overall levels of mRNA decrease sharply, as measured by an oligo(dT) hybridization assay (not shown). At the treatment doses, all three compounds induce apoptosis by 24 hr, as determined by an annexin V assay (FIG. 4A), which measures appearance of cell-surface phosphatidyl serine as an apoptotic marker. Thioacetamide induces the greatest response in this assay. Another assay, which measures caspase-3 levels, shows that only in thioacetamide-treated cells at 24 hr is there significant activation of this apoptotic pathway (FIG. 4B).

Prior to performing expression profiling, we optimized cDNA array hybridization and wash conditions, using as a benchmark the gene for lactate dehydrogenase-A (LDH-A). We had previously observed a 4- to 9-fold down-regulation of this gene under each of our treatment conditions (see Example 1). Using samples from cells treated for 24 hr with 20 mM acetaminophen, we performed overnight hybridizations, followed by washes at various stringencies prior to exposure to storage phosphor screens. The intensities of spots corresponding to the LDH-A gene on the arrays were determined and, following normalization (discussed below), the expression change upon acetaminophen treatment was calculated. The expression ratios observed using different wash stringencies were compared to the ratios observed in Northern blot and quantitative RT-PCR assays (Table 9). With the two lower stringency washes, little if any apparent change in LDH-A gene expression was observed, in contrast to the six-fold decrease seen in the PCR and Northern blot measurements. A down-regulation of 11-fold was observed, however, on arrays washed with 0.5× SSC at 65° C. At the highest stringency condition, 0.25× SSC at 65° C., we observed severely reduced spot intensities and significantly fewer detectable spots, which made quantitation difficult. As a result, we chose the 0.5× SSC, 65° C. wash for subsequent experiments. We also examined hybridization time, but found no apparent difference between arrays hybridized for 72 hr and those hybridized overnight. Consequently, overnight hybridization was used in our standard protocol. Increasing the amount of mRNA used for cDNA synthesis also had no effect on the quality of the data (not shown).

In the DD PCR experiments described in Example 1, we observed different temporal patterns of expression among genes affected by toxic treatments. By performing expression profiling at only a single time point, there is the risk of identifying only a subset of the genes affected. In order to avoid this problem in the present study, we performed detailed time course experiments for each compound, with nine treatment times ranging from 1 to 24 hr, with an associated untreated control at each time point. For each time point, mRNA was isolated from cells and used as template for the synthesis of radiolabeled cDNA, which was hybridized to the arrays. For each sample, we performed four replicate sets of array hybridizations.

Following spot quantitation using image processing software, spot intensities were normalized by applying a local regression algorithm that uses the intensities of all spots on the array to calculate a smooth normalization function that is applicable throughout the signal intensity range. This normalization technique performs better than methods based on applying a single normalization factor to the entire set of spots, derived either from comparison of median intensity values or expression of “housekeeping genes”. The normalized expression values for each set of treated and control arrays were compared, and expression changes significant at 95% confidence were identified using a locally-smoothed approximation of the variance. Background was estimated by visual inspection of array images. Spots with normalized intensities below the background threshold (0.0002 on the normalized expression scale) in both control and treated samples were ignored. Approximately 1,000 spots were above background on each array.

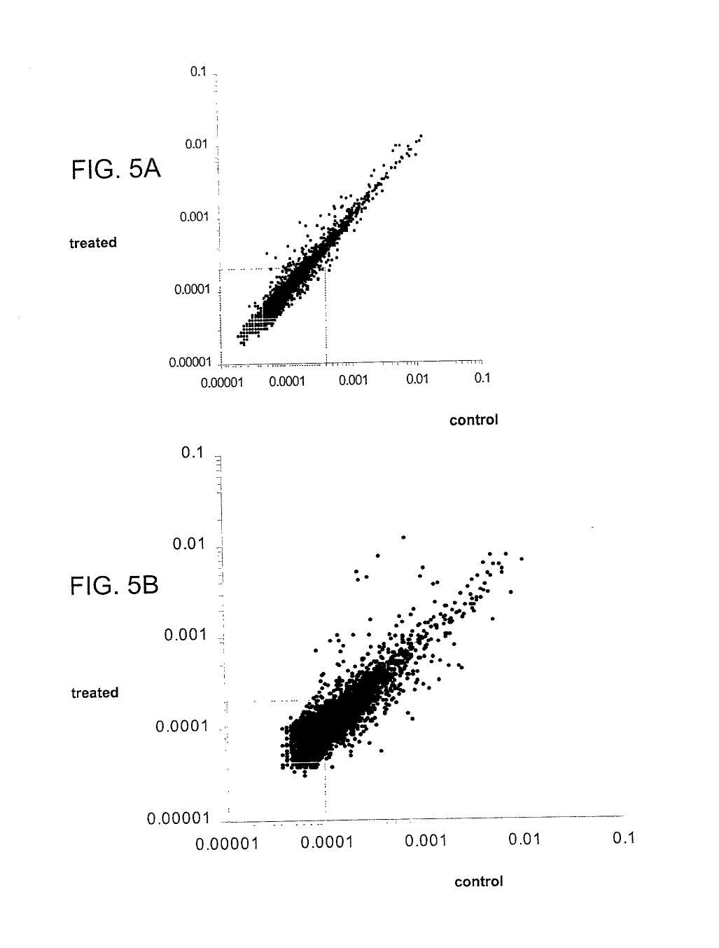

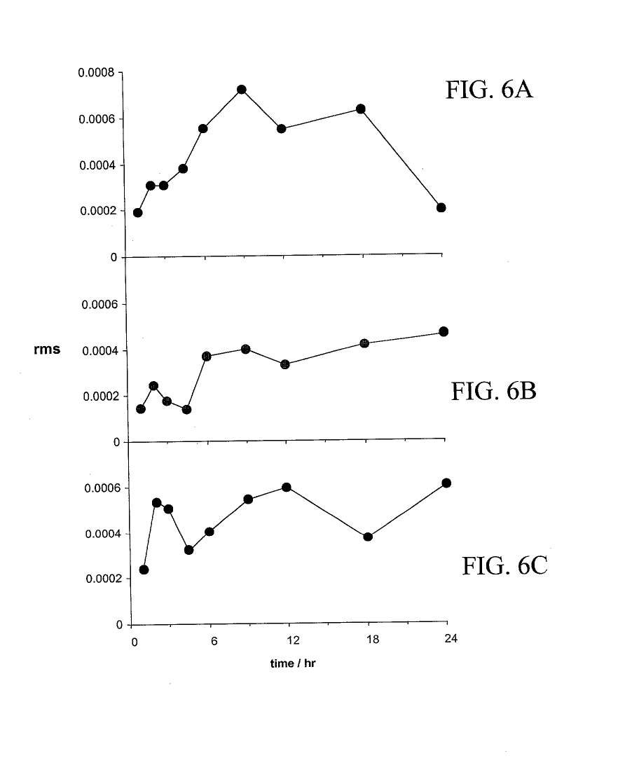

As an example of the distribution of spot intensities following normalization, FIGS. 5A and 5B compare plots of control vs. treated values for acetaminophen treatment at 2 and 18 hr. In this example, greater modulation in expression is observed at the later time point (18 hr, FIG. 5B) than at the earlier one (2 hr, FIG. 5A), both with respect to the number of genes affected and the magnitude of the expression changes. An examination of the root-mean-square (rms) differences between control and treated intensities, which provides a measure of global expression changes without regard to direction, indicates that with acetaminophen, differential gene expression reaches a peak between 6 and 18 hr (FIG. 6A). Caffeine elicits few changes until 6 hr, after which overall differential expression is fairly constant (FIG. 6B). Such trends are less clear with thioacetamide treatment, where a high degree of differential expression is observed both at early and late time points (FIG. 6C).

In analyzing expression data from time course experiments, we avoided imposing an arbitrary fold-change threshold as a means of identifying changes of interest. Rather, we concentrated our analysis on genes with a statistically significant (p<0.05) change in expression in three or more adjacent time points. This criterion limited the number of genes of interest to 258 for acetaminophen, 215 for thioacetamide, and 158 for caffeine.

For each treatment, we used cluster analysis to classify the genes based on their temporal patterns of differential expression. Roughly two-thirds of the observed changes in expression are down-regulations. This trend is consistent with the previous results using differential display-PCR (see Example 1), where approximately 75% of the confirmed gene expression changes were down-regulations. We observe a variety of distinct temporal expression patterns, which are distinguished from one another primarily by three factors: the overall direction of the expression change (up or down), the time at which the change begins to occur (early to midway through the time course), and the degree to which the change persists through to the last time point.

There is considerable overlap between the genes affected by the different treatments. Of 434 genes, 81 appear in both the acetaminophen and caffeine sets, 93 are common to acetaminophen and thioacetamide, and 71 are affected by both caffeine and thioacetamide. At a more detailed level, some clusters are more similar than others in terms of the genes that comprise them. For example, caffeine cluster 3 shares 23 genes with thioacetamide cluster 8, which is, at 95% confidence, more than the 8 that would be expected based on random distributions. Thus, these two clusters are positively correlated. Conversely, caffeine cluster 3 has no genes in common with thioacetamide cluster 1, although 4 would be expected if the genes were distributed randomly; these clusters are negatively correlated. In general, when clusters are positively correlated, both show gene expression changes in the same direction. When clusters are negatively correlated, invariably one contains up-regulated genes, the other down-regulated. These observations indicate that there are similarities in the transcriptional responses to the toxicants examined in this study.

A few clusters do not show a positive correlation with any other cluster in the pairwise comparisons. A striking example is thioacetamide cluster 2. Of the 33 genes that comprise this cluster, only 2 are affected by either of the other treatments. Thus, the temporal pattern of expression exhibited by this cluster appears to be fairly specific for thioacetamide. The genes in this cluster show up-regulation early in the time course, generally by 2 hr. These genes may indicate an early response specific to thioacetamide, and perhaps to other compounds acting through a similar mechanism of cytotoxicity.

A total of 48 genes are affected by all three toxicants. Of these, 44 genes are modulated in the same direction by each of the three treatments. The degree of overlap is greater (p<0.01) than would be expected if the expression differentials arose through completely independent mechanisms. This observation is consistent with the hypothesis that the overlap in expression changes is due to real similarities in the transcriptional responses of the cell to these three toxicants. The 44 genes in the common set are listed in Table 12. These genes tend to be those for which the expression changes occur in the later time points; clusters characterized by early expression differentials are underrepresented.

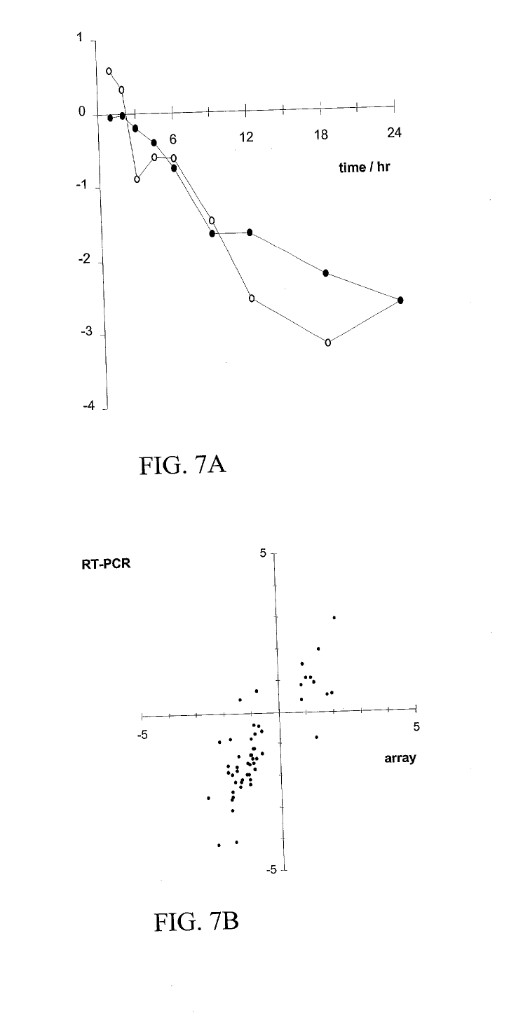

In order to test the accuracy of the array results, we performed two sets of quantitative RT-PCR experiments. First, we used the TaqMan assay to quantitate LDH-A gene expression as a function of time in response to acetaminophen. This comparison allowed us to assess the ability of the array method to reliably measure a range of expression changes, using a single gene. As indicated in FIG. 7A, the two assays are in close agreement. In the second set of experiments, we designed specific PCR primers and TaqMan probes to each of the genes listed in Table 12, as well as to other selected genes. We performed quantitative RT-PCR using the acetaminophen samples, generally at the time point giving the largest fold change for each particular gene (Table 10). This experiment allowed us to assess the degree to which the results may be influenced by cross-hybridization or by spotting of the wrong clone on the arrays. Cross-hybridization could occur with highly homologous genes, even with our high stringency wash conditions. Spotting of the wrong clone is expected to occur rarely; however, the relatively frequent occurrence of incorrect sequence among IMAGE clones (10-15% in our experience; data not shown) does raise this as a possibility. In fact, at least one of the genes listed in Table 10 that showed poor agreement between array and RT-PCR data, TTF-1 interacting peptide 21, appears to fall into this category. On the arrays, we observed a 2.6-fold up-regulation of this gene in response to acetaminophen at 12 hr; however, the RT-PCR assay indicated a down-regulation of close to 2-fold. We obtained the IMAGE clone corresponding to this gene and sequenced it. We found that the sequence did not correspond to TTF-1 interacting peptide 21, raising the possibility that the clone spotted on the array was also incorrect. Another potential problem arises from errors in the sequence databases. We carefully examined all our designed probes to ensure a perfect match against multiple ESTs derived from the genes of interest so as to avoid problems that can arise with mismatches (see e.g., Hildebrand et al., Toxicol. in Vitro 13:561-565 (1999); Stenman et al. Nature Biotech;. 17:720-722 (1999)). For one gene (EST R51835), we were unable to design an acceptable probe based on the limited sequence data available.

In general, the agreement between the expression ratios derived from the arrays and those obtained from PCR quantitation was quite high (FIG. 7B). The direction of change was confirmed in about 90% of cases, and in most instances the magnitude of change reported by the two assays was quite similar. This high degree of confirmation is likely to be attributable to the strict criteria we used to select genes for confirmation. The genes we tested in the TaqMan assay were selected because they showed statistically significant modulation in three adjacent time points, using data derived from quadruplicate array hybridizations. Moreover, in most cases, these criteria were met in response to three separate treatments. Had the genes tested in the TaqMan assay been chosen based on fewer replicates, fewer time points, or fewer treatments, we expect that the confirmation rate would have been lower.

One of the expression changes that failed to confirm involved metallothionein-1G. The array data indicated an 18-fold induction by acetaminophen at the 24-hr time point, whereas the TaqMan assay, which should provide a more sensitive measurement, failed to detect expression in either the control or the treated sample. Since this gene is a member of a highly homologous gene family, we suspected that cross-hybridization on the arrays was producing misleading results. To test this possibility, we designed specific TaqMan probes to each of the five metallothionein genes present on the array. In both the acetaminophen and thioacetamide samples, we observed significant up-regulation of all five forms on the arrays, with 14- to 23-fold changes in expression. In the PCR assay, however, four of the forms, including 1G, were either undetectable or present at very low levels, not expected to be detectable on the arrays. Metallothionein-1H, however, showed a >1000-fold induction, going from undetectable in the control samples to highly expressed in the treated samples (Table 11). These results indicate that cross-hybridization between these genes, which share approximately 85% identity in regions, accounted for the array results, even though only one form was actually induced to the extent indicated on the arrays. The fact that only one of the five forms appears on the common list of genes appears to be due to the relatively low degree of up-regulation induced by caffeine; for only one of the forms did the apparent expression change happen to meet the criteria for inclusion on the list.

The genes affected in common by the three treatments comprise a diverse set of functions, indicating effects on a variety of cell processes (Table 12). As we observed in our DD-PCR study, a number of genes involved in basic cellular metabolism are down-regulated by all three treatments (see Example 1). Among these “housekeeping genes” are several that encode proteins involved in mitochondrial energy production, including cytochrome c-1 and individual subunits of the pyruvate dehydrogenase, F₁F₀-ATPase synthase, and ubiquinol-cytochrome c reductase complexes. This down-regulation of genes involved in energy production and other basic cellular reactions may reflect the general attenuation of cell function as cells enter apoptosis.

Two apoptosis-related genes are modulated by all three treatments. The gene encoding the apoptotic chromatin condensation inducer in the nucleus (acinus) is up-regulated. This gene encodes a caspase-activated protein that is necessary for the chromatin condensation that occurs in apoptosis (see e.g., Sahara et al., Nature 401:168-173 (1999)). Conversely, DAD1 (defender against cell death 1), the loss of which has been shown to trigger apoptosis in hamster cells (see e.g., Nakashima et al., Mol. Cell Biol. 13:6367-6374 (1993)), is down-regulated in all three treatments.

We observe down-regulation of at least two genes involved in protein transport, the homologs of the yeast SEC 13 and SEC23 genes. In yeast, these genes encode proteins required for the formation of vesicles from the endoplasmic reticulum and their transport to the Golgi (see e.g., Paccaud et al., Mol. Biol. Cell 7:1535-1546 (1996); Swaroop et al., Hum. Mol. Genet. 3:1281-1286 (1994)). In addition, the KIAA0917 gene is down-regulated in all three treatments. This gene is homologous to a rat vesicle transport-related protein (see e.g., Nagase et al., DNA Res. 5:355-364 (1998)).

Although most of the genes affected by all three treatments are not known “stress genes,” several do fall into this category. The gene for XP-C repair complementing protein, which is involved in DNA excision repair (see e.g., Masutani et al., EMBO J. 13:1831-1843 (1994)), is down-regulated. Two forms of glutathione-S-transferase, which is involved in cellular redox balance, is also down-regulated. Metallothionein-1H, as discussed above, is strongly induced by acetaminophen and thioacetamide, and to a much lesser extent by caffeine.

It is interesting to compare the results presented here with those we obtained by DD-PCR coupled with a dot blot confirmation assay. Of the nine genes identified by DD-PCR and shown to be modulated by all three toxicants, only three were present on the cDNA array. All three of these genes were down-regulated at 24 hr in the DD-PCR study. For two of these genes, encoding lactate dehydrogenase-A and pyruvate dehydrogenase, the results are confirmed in the present study. The third gene, for transforming growth factor-beta type III receptor, was expressed below background and therefore could not be quantitated on the arrays.

In addition, two genes identified on the arrays as down-regulated by all three treatments had been found in the DD-PCR study to be affected by at least one treatment. One of these genes, encoding ubiquinol-cytochrome c reductase core protein II, had been seen in Example 1 to be down-regulated by caffeine and thioacetamide, but not by acetaminophen, at the 24 hr time point, the only time point used in that study. In fact, the arrays support this result, as the expression level returns to normal by 24 hr with acetaminophen treatment. The other gene, for acetyl-coenzyme A acetyltransferase 2, appears to be down-regulated by all three treatments at 24 hr on the arrays. In the DD-PCR study, the down-regulation was confirmed only in acetaminophen and caffeine samples, even though the effect was originally identified with thioacetamide treatment.

Comparison between the DD-PCR study and the probe array study indicates that there is good agreement between the two methods, and indicates that open and closed systems are complementary. The open system was able to identify some effects that the closed system could not. However, the arrays, with their higher throughput, allowed us to perform time courses that uncovered a greater number of genes with a higher rate of confirmation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes to the same extent as if each individual publication, patent or patent application were specifically and individually indicated to be so incorporated by reference.

TABLE 8
Experimental strategy

| STEP | COMMENTS |
|---|---|
| 1. Treatment of cells | HepG2 cells were treated with toxic doses of |
| | acetaminophen, caffeine and thioacetamide for 1, 2, 3, |
| | 4.5, 6, 9, 12, 18 and 24 hr. |
| 2. Isolation of mRNA | mRNA from treated and control cells was prepared by |
| | affinity purification on oligo(dT) cellulose |
| 3. Preparation of target nucleic acid | α-³³P-labeled cDNA was prepared by reverse |
| | transcription |
| 4. Hybridization to arrays | Labeled cDNA was hybridized to 5,000-gene cDNA |
| | arrays for 16-18 hrs |
| 5. High stringency washes | High stringency washes were carried out in 0.5X SSC |
| | at 65° C. to reduce background and cross-hybridization |
| 6. Spot quantitation | Array images were acquired by phosphorimaging and |
| | quantitated using spot detection software |
| 7. Data normalization | Normalization by local regression was applied to |
| | quadruplicate sets of arrays to allow comparison |
| | between control and treated |
| 8. Identification of differentially | Genes were identified with statistically significant |
| expressed genes | expression changes in three adjacent time points in |
| | each of the three treatments |
| 9. Confirmation assays | Genes of interest were examined by quantitative RT- |
| | PCR |

TABLE 9
Optimization of wash conditions used with cDNA filter arrays

| | | | | Observed LDH-A |
|---|---|---|---|---|
| | WASH CONDITIONS¹ | | | expression ratio |
| Assay method | X SSC | T/° C. | n | (treated/control)² |
| TaqMan RT-PCR | NA | NA | 2 | 0.16 |
| Northern blot | 0.1 | 65 | 1 | 0.16 |
| cDNA array | 2 | 50 | 2 | 0.88 |
| | 1 | 65 | 3 | 1.3 |
| | 0.5 | 65 | 2 | 0.09 |
| | 0.25 | 65 | 2 | 0.26 |

¹Highest stringency wash. NA, not applicable.
²Expression of lactate dehydrogenase-A was measured following 24 hr treatment of HepG2 cells with 20 mM acetaminophen.

TABLE 10
Expression ratios of selected genes in response to 20 mM acetaminophen measured
by array and RT-PCR

| | | Expression ratio¹ | | |
|---|---|---|---|---|
| GenBank | Gene | Time (hr) | Array | RT-PCR |
| AA446819 | Ornithine aminotransferase (gyrate atrophy) | 12 | 4.4 | 7.5 |
| H93328 | Putative cyclin G1 interacting protein | 12 | 2.9 | 3.9 |
| H75861 | Acinus | 18 | 1.9 | 2.8 |
| H20652 | KIAA0069 | 12 | 2.3 | 2.1 |
| AA232856 | DNA topoisomerase I | 18 | 2.0 | 2.1 |
| R84893 | KIAA0220 | 12 | 2.5 | 1.9 |
| W31074 | Fatty-acid-coenzyme A ligase, long-chain 3 | 6 | 1.8 | 1.8 |
| H73961 | Actin-related protein ⅔ complex, subunit 3 | 9 | 0.59 | 1.6 |
| AA233079 | Insulin-like growth factor binding protein 1 | 12 | 3.9 | 1.5 |
| R51607 | Translation initiation factor eIF1 (A121/SUI1) | 12 | 3.5 | 1.4 |
| W74293 | ESTs, highly similar to laminin B | 12 | 1.8 | 1.3 |
| N53133 | EST | 24 | 0.38 | 1.3 |
| AA127685 | Multispanning membrane protein | 9 | 0.53 | 0.75 |
| AA455281 | Defender against cell death 1 | 9 | 0.60 | 0.73 |
| R78585 | Calumenin | 12 | 0.64 | 0.66 |
| AA453335 | Thioredoxin reductase 1 | 4.5 | 0.54 | 0.62 |
| H92821 | TTF-1 interacting peptide 21 | 12 | 2.6 | 0.57 |
| H73484 | EST | 24 | 0.49 | 0.57 |
| N49629 | Diubiquitin | 12 | 0.29 | 0.56 |
| AA448396 | Heat shock 10 kD protein 1 (chaperonin 10) | 18 | 0.22 | 0.54 |
| AA406332 | COPII protein, SEC23p homolog | 6 | 0.53 | 0.46 |
| AA486324 | Proteasome activator subunit 3 (PA28 gamma; Ki) | 4.5 | 0.51 | 0.46 |
| H68845 | Thioredoxin-dependent peroxide reductase 1 | 12 | 0.64 | 0.41 |
| AA456400 | Adenylosuccinate lyase | 12 | 0.49 | 0.40 |
| R01118 | Squalene epoxidase | 24 | 0.48 | 0.40 |
| AA456474 | Apolipoprotein C-II | 24 | 0.35 | 0.39 |
| R12802 | Ubiquinol-cytochrome c reductase core protein II | 12 | 0.55 | 0.37 |
| H90815 | Corticosteroid binding globulin | 18 | 0.50 | 0.37 |
| AA486312 | Cyclin-dependent kinase 4 | 12 | 0.52 | 0.33 |
| AA489678 | XP-C repair complementing protein | 12 | 0.44 | 0.33 |
| AA447774 | Cytochrome c-1 | 9 | 0.47 | 0.32 |
| AA521401 | Pyruvate dehydrogenase (lipoamide) beta | 9 | 0.27 | 0.31 |
| H38623 | F₁F₀-ATPase synthase f subunit | 24 | 0.34 | 0.30 |
| W33012 | Transcription factor Dp-1 | 9 | 0.53 | 0.29 |
| H94897 | Human chromosome 3p21.1 gene sequence | 9 | 0.34 | 0.28 |
| T65902 | Splicing factor, arginine/serine-rich 1 | 9 | 0.27 | 0.27 |
| AA496784 | SEC13 (S. cerevisiae)-like 1 | 12 | 0.45 | 0.26 |
| R28294 | Glycine cleavage system protein H | 18 | 0.43 | 0.26 |
| AA441895 | Glutathione-S-transferase like | 9 | 0.30 | 0.26 |
| N79230 | MAC30 | 18 | 0.47 | 0.23 |
| R54424 | Glutamate dehydrogenase | 18 | 0.38 | 0.23 |
| AA495936 | Microsomal glutathione-S-transferase | 18 | 0.31 | 0.23 |
| AA402960 | Ring finger protein 5 | 18 | 0.37 | 0.22 |
| AA458965 | Natural killer cells transcript 4 | 24 | 0.32 | 0.22 |
| AA028034 | KIAA0917 (vesicle transport-related protein) | 6 | 0.47 | 0.21 |
| T47454 | Tissue factor pathway inhibitor | 18 | 0.36 | 0.20 |
| H55921 | Ribosomal protein S6 kinase, 90 kD, polypeptide 3 | 9 | 0.30 | 0.18 |
| AA143509 | Pyrroline-5-carboxylate synthetase | 12 | 0.30 | 0.16 |
| H05914 | Lactate dehydrogenase-A | 24 | 0.16 | 0.16 |
| T65907 | Farnesyl diphosphate synthase | 18 | 0.29 | 0.15 |
| R25823 | Acetyl-coenzyme A acetyltransferase 2 | 12 | 0.29 | 0.12 |
| T60223 | Ribonuclease, RNase A family, 4 | 18 | 0.20 | 0.057 |

¹Treated/control.

TABLE 11
Observed expression ratios for the metallothionein
gene family measured by cDNA array and RT-PCR¹

| | | Acetaminophen | | Thioacetamide | |
|---|---|---|---|---|---|
| | | (18 hr) | | (24 hr) | |
| Gene | GenBank | Array | RT-PCR² | Array | RT-PCR² |
| MT-1B | H72722 | 16 | ND | 15 | ND |
| MT-1G | H53340 | 18 | ND | 15 | 3.1 |
| MT-1H | H77766 | 23 | >1000 | 16 | >1000 |
| MT-1L | N80129 | 21 | ND | 14 | ND |
| MT-2 | R16596 | 18 | 3.2 | 15 | 7.4 |

¹Expression ratios are treated/control.
²ND, not detectable in either control or treated. MT-1H was not detectable in the control samples.

TABLE 12
Nucleic acids identified by probe array to be similarly affected by all three treatments

| GenBank | UniGene | Name² |
|---|---|---|
| H93328 | Hs.92374 | *Putative cyclin G1 interacting protein |
| W74293 | Hs.27375 | *EST, highly similar to laminin B1 |
| AA100612 | Hs.71827 | †KIAAD112 |
| W31074 | Hs.243925 | *Fatty-acid -coenzyme A ligase, long-chain 3 |
| R84893 | Hs.110613 | *KIAA0220 |
| H20652 | Hs.75249 | *KIAA0069 |
| H75861 | Hs.227133 | *Acinus |
| R51607 | Hs.150580 | *Translation Initiation factor eIF1(A12/SUI1) |
| AA446819 | Hs.75485 | *Ornithine aminotransferase (gyrate atrophy) |
| AA233079 | Hs.102122 | *Insulin-like growth factor binding protein 1 |
| H53340 | Hs.173451 | †Metallothionein-1G |
| H38623 | Hs.155751 | *F₁F₀-ATPase synthase ƒ subunit |
| AA402960 | Hs.216354 | *Ring finger protein 5 |
| H73484 | Hs.9601 | *EST |
| AA489678 | Hs.178658 | *XP-C repair complementing protein |
| RO1118 | Hs.71465 | *Squalene epoxidase |
| AA495936 | Hs.790 | *Microsomal glutathione-S-transferase 1 |
| AA455281 | Hs.82890 | *Defender against cell death 1 |
| AA034268 | | †EST |
| AA406332 | Hs.92962 | *COPII protein, SEC23p homolog |
| AA028034 | Hs.27023 | *KIAA0917 (vesicle transport-related protein) |
| H90815 | Hs.1305 | *Corticosteroid binding globulin |
| R78585 | Hs.7753 | *Calumenin |
| R12802 | Hs.173554 | *Ubiquinol-cytochrome c reductase core protein II |
| AA496784 | Hs.227949 | *SEC13 (S. cerevisiae)-like 1 |
| R51835 | Hs.167371 | EST |
| H94897 | Hs.82837 | *Human chromosome 3p21.1 gene sequence |
| AA441895 | Hs.11465 | *Glutathione-S-transferase-like |
| T60223 | Hs.169617 | *Ribonuclease, RNase A family, 4 |
| W33012 | Hs.79353 | *Transcription factor Dp-1 |
| H73961 | Hs.6895 | †Actin-related protein ⅔ complex, subunit 3 |
| N79230 | Hs.199695 | *MAC30 |
| AA486312 | Hs.95577 | *Cyclin-dependent kinase 4 |
| AA127685 | Hs.91586 | *Multispanning membrane protein |
| T65902 | Hs.73737 | *Splicing factor, arginine/serine-rich 1 |
| AA447774 | Hs.697 | *Cytochrome c-1 |
| H05914 | Hs.2795 | *Lactate dehydrogenase-A |
| N53133 | Hs.8215 | †EST |
| AA143509 | Hs.114366 | *Pyrroline-5-carboxylate synthetase |
| R54424 | Hs.77508 | *Glutamate dehydrogenase |
| AA521401 | Hs.979 | *Pyruvate dehydrogenase (lipoamide) beta |
| H55921 | Hs.173965 | *Ribosomal protein S6 kinase, 90 kD, polypeptide 3 |
| R25823 | Hs.4112 | *Acetyl-coenzyme A acetyltransferase 2 |
| AA486324 | Hs.152978 | *Proteasome activator subunit 3 (PA28 gamma; Kᵢ) |

²Genes are grouped into up-regulated (above dividing line) and down-regulated (below dividing line). Clones tested and confirmed by RT-PCR are indicated by asterisks (*); clones that failed to confirm are indicated by daggers (†).

### APPENDIX A

| Acc # | title |
|---|---|
| AA100612 | Human mRNA for KIAA0112 gene, partial cds |
| AA233079 | INSULIN-LIKE GROWTH FACTOR BINDING PROTEIN 1 PRECURSOR |
| AA446819 | Ornithine aminotransferase (gyrate atrophy) |
| H20652 | Human mRNA for KIAA0069 gene, partial cds |
| H75861 | ESTs, Weakly similar to coded for by C. elegans cDNA yk93e11.5 [C. elegans] |
| H93328 | Human putative cyclin G1 interacting protein mRNA, partial sequence |
| R51607 | Similar to PROTEIN TRANSLATION INITIATION FACTOR SUI1 HOMOLOG |
| R84893 | Homo sapiens Chromosome 16 BAC clone CIT987-SKA-589H1-complete genomic sequence |
| W31074 | ESTs, Weakly similar to LONG-CHAIN-FATTY-ACID--COA LIGASE 1 [Saccharomyces cerevisiae] |
| W74293 | ESTs, Highly similar to HYPOTHETICAL 66.9 KD PROTEIN R07B1.8 IN CHROMOSOME X |
| | [Caenorhabditis elegans] |
| AA453335 | Thioredoxin reductase |
| AA485036 | Human mRNA for KIAA0201 gene, complete cds |
| AA293819 | Human transcription factor NFATx mRNA, complete cds |
| AA456028 | Human geranylgeranyl transferase type II beta-subunit mRNA, complete cds |
| AA460115 | Ornithine decarboxylase 1 |
| R61674 | Human protein tyrosine phosphatase PTPCAAXI (hPTPCAAX1) mRNA, complete cds |
| R62285 | ESTs |
| T68518 | Human mRNA for PIMT isozyme I, complete cds |
| W52208 | ESTs, Highly similar to deduced protein product shows significant homology to coactosin |
| | from Dictyostelium discoideum [H. sapi |
| AA011215 | Spermidine/spermine N1-acetyltransferase |
| AA430035 | Human MEK5 mRNA, complete cds |
| AA456109 | Human scaffold protein Pbp1 mRNA, complete cds |
| AA478436 | Human SWI/SNF complex 60 KDa subunit (BAF60b) mRNA, complete cds |
| AA481758 | DNAJ PROTEIN HOMOLOG 1 |
| R20379 | Eukaryotic translation elongation factor 2 |
| R39954 | Homo sapiens post-synaptic density protein 95 (PSD95) mRNA, complete cds |
| AA001614 | Insulin receptor |
| AA029041 | ESTs, Highly similar to DEVELOPMENTAL PROTEIN SEVEN IN ABSENTIA [Drosophila melanogaster] |
| AA083032 | H. sapiens mRNA for cyclin G1 |
| AA126356 | Calnexin |
| AA397813 | CDC28 protein kinase 2 |
| AA446251 | Laminin B1 chain |
| AA448261 | High mobility group (nonhistone chromosomal) protein isoforms I and Y |
| AA464152 | Human quiescin (Q6) mRNA, partial cds |
| AA478724 | Insulin-like growth factor binding protein 6 |
| AA486085 | THYMOSIN BETA-10 |
| AA486138 | Vacuolar H+ ATPase proton channel subunit |
| AA486626 | Poly(A)-binding protein-like 1 |
| AA488721 | Transferrin receptor (p90, CD71) |
| AA489839 | Human mRNA for KIAA0127 gene, complete cds |
| AA490213 | Human mRNA for Tob, complete cds |
| AA495944 | Human WD repeat protein HAN11 mRNA, complete cds |
| AA598601 | Human growth hormone-dependent insulin-like growth factor-binding protein mRNA, complete cds |
| AA598776 | Human p55CDC mRNA, complete cds |
| AA598950 | Cathepsin B |
| H02158 | Heterogeneous nuclear ribonucleoprotein K |
| H14841 | ATPase, Nat+/K+ transporting, beta 2 polypeptide |
| H63706 | ESTs, Weakly similar to CASEIN KINASE I HOMOLOG HRR25 [Saccharomyces cerevisiae] |
| H64324 | Human guanine nucleotide exchange factor mRNA, complete cds |
| H71868 | Hexosaminidase B (beta polypeptide) |
| H81048 | ESTs |
| H82706 | Inhibitor of DNA binding 2, dominant negative helix-loop-helix protein |
| H89996 | Human transcriptional repressor (CTCF) mRNA, complete cds |
| H93550 | ESTs |
| N54596 | Insulin-like growth factor 2 (somatomedin A) |
| N59542 | ESTs, Weakly similar to coded for by C. elegans cDNA CEESW58F [C.elegans] |
| N59721 | ESTs, Highly similar to GLIA DERIVED NEXIN PRECURSOR [Homo sapiens] |
| N95657 | ESTs, Highly similar to HYPOTHETICAL 63.5 KD PROTEIN ZK353.1 IN CHROMOSOME III |
| | [Caenorhabditis elegans] |
| R02166 | ESTs, Moderately similar to !!!! ALU SUBFAMILY J WARNING ENTRY !!!! [H.sapiens] |
| R19878 | Human reelin (RELN) mRNA, complete cds |
| R31168 | Human hbc647 mRNA sequence |
| R32952 | S-100P PROTEIN |
| R44334 | Human 90 kD heat shock protein gene, complete cds |
| R48796 | Integrin, alpha L (antigen CD11A (p180), lymphocyte function-associated antigen 1; alpha polypeptide) |
| R53889 | Human non-histone chromosomal protein HMG-14 mRNA, complete cds |
| R54097 | Human translational initiation factor 2 beta subunit (eIF-2-beta) mRNA, complete cds |
| R61295 | Human ADP/ATP translocase mRNA, 3′ end, clone pHAT8 |
| R63219 | EST |
| R84407 | ESTs |
| R88741 | ESTs, Moderately similar to proliferation potential-related protein [M. musculus] |
| R93829 | H. sapiens NAP (nucleosome assembly protein) mRNA, complete cds |
| R93875 | HETEROGENEOUS NUCLEAR RIBONUCLEOPROTEINS C1/C2 |
| R94601 | ESTs |
| R98008 | CAG-isl 7 {trinucleotide repeat-containing sequence} [human, pancreas, mRNA Partial, 701 nt] |
| T51689 | Human hybrid receptor gp250 precursor mRNA, complete cds |
| T69926 | Myosin, heavy polypeptide 9, non-muscle |
| T70503 | PLASMA-CELL MEMBRANE GLYCOPROTEIN PC-1 |
| W04152 | ESTs |
| W67174 | Integrin, beta 1 (fibronectin receptor, beta polypeptide, antigen CD29 includes MDF2, MSK12) |
| W67323 | Human mRNA for RBP-MS/type 1, complete cds |
| H53340 | Human (clone 14V5) metallothionein-IG (MT1G) gene, complete cds |
| H72722 | Human metallothionein I-B gene |
| H77766 | H. sapiens mRNA for metallothionein |
| N80129 | Metallothionein 1L |
| R16596 | ESTs, Highly similar to METALLOTHIONEIN-II [H. sapiens] |
| AA495846 | TRANSFORMING PROTEIN RHOB |
| R06309 | ESTs |
| AA598794 | Connective tissue growth factor |
| AA028034 | ESTs, Highly similar to rsly1p [R.norvegicus] |
| AA034268 | ESTs, Highly similar to NADH-UBIOUINONE OXIDOREDUCTASE B17 SUBUNIT [Bos taurus] |
| AA127685 | Human multispanning membrane protein mRNA, complete cds |
| AA143509 | Pyrroline-5-carboxylate synthetase (glutamate gamma-semialdehyde synthetase) |
| AA402960 | Human HLA class III region containing NOTCH4 gene, partial sequence, homeobox |
| | PBX2 (HPBX) gene, receptor for advanced glycosylation end products (RAGE) gene, complete cds, |
| | and 6 unidentified cds |
| AA406332 | H. sapiens mRNA for Sec23A isoform, 2748bp |
| AA441895 | Human glutathione-S-transferase homolog mRNA, complete cds |
| AA447774 | Cytochrome c1 |
| AA455281 | DEFENDER AGAINST CELL DEATH 1 |
| AA486312 | Human cyclin-dependent protein kinase mRNA, complete cds |
| AA486324 | Human Ki nuclear autoantigen mRNA, complete cds |
| AA489678 | Human mRNA for XP-C repair complementing protein (p58/HHR23B), complete cds |
| AA495936 | GLUTATHIONE 5-TRANSFERASE, MICROSOMAL |
| AA496784 | Human (chromosome 3p25) membrane protein mRNA |
| AA521401 | Pyruvate dehydrogenase (lipoamide) beta |
| H05914 | Human mRNA for lactate dehydrogenase-A (LDH-A, EC 1.1.1.27) |
| H38623 | ESTs, Highly similar to GLYCYLPEPTIDE N-TETRADECANOYLTRANSFERASE [Homo sapiens] |
| H55921 | Human insulin-stimulated protein kinase I (ISPK-1) mRNA, complete cds |
| H73484 | ESTs, Weakly similar to B0334.4 [C. elegans] |
| H73961 | EST |
| H90815 | Corticosteroid binding globulin |
| H94897 | Human chromosome 3p21.1 gene sequence |
| N53133 | ESTs, Moderately similar to M-phase phosphoprotein 4 [H. sapiens] |
| N79230 | Human MAC3O mRNA, 3′ end |
| R01118 | Homo sapiens mRNA for squalene epoxidase, complete cds |
| R12802 | Human cytochrome bc-1 complex core protein II mRNA, complete cds |
| R25823 | T-COMPLEX PROTEIN 1, ALPHA SUBUNIT |
| R51835 | unknown EST |
| R54424 | Human liver glutamate dehydrogenase mRNA, complete cds |
| R78585 | ESTs, Highly similar to RETICULOCALBIN PRECURSOR [Mus musculus] |
| T60223 | Ribonuclease L (2′,5′-oligoisoadenylate synthetase-dependent) |
| T65902 | PRE-MRNA SPLICING FACTOR SF2, P33 SUBUNIT |
| W33012 | Home sapiens E2F-related transcription factor (DP-1) mRNA, complete cds |
| AA022627 | ESTs, Highly similar to NADH-UBIQUINONE OXIDOREDUCTASE SUBUNIT B14.5A [Bos taurus] |
| AA449048 | ESTs, Highly similar to M-phase phosphoprotein 4 [H. sapiens] |
| AA452916 | Lysyl oxidase |
| AA453859 | Alcohol dehydrogenase 5 chi subunit (class III) |
| AA481076 | Human mitotic feedback control protein Madp2 homolog mRNA, complete cds |
| H08642 | Dentatorubral-pallidoluysian atrophy |
| H51066 | H. sapiens OB-RGRP gene |
| H52001 | Flavin containing monooxygenase 5 |
| H53274 | Human mRNA for histamine N-methyltransferase, complete cds |
| H65066 | Visinin-like 1 |
| R09815 | ESTs, Highly similar to 26S PROTEASE REGULATORY SUBUNIT 8 [Home sapiens] |
| R22274 | Human mRNA for phosphoethanolamine cytidylyltransferase, complete cds |
| R44822 | Human mRNA for phosphoribosypyrophosphate synthetase-associated protein 39, complete cds |
| R78514 | ESTs, Highly similar to VESICULAR INTEGRAL-MEMBRANE PROTEIN VIP36 PRECURSOR |
| | [Canis familiaris] |
| W00959 | Hepatic leukemia factor |
| H23963 | EST |
| R52654 | Cytochrome c-1 |
| AA411407 | Signal recognition particle 19 kD protein |
| AA424807 | Human mRNA for KIAA0107 gene, complete cds |
| AA428518 | H. sapiens cl.1042 mRNA of DEAD box protein family |
| AA454585 | Splidng factor, arginine/serine-rich 2 |
| AA465593 | PROTEASOME COMPONENT C8 |
| AA465611 | Human mRNA for KIAA0190 gene, partial cds |
| AA487893 | TUMOR-ASSOCIATED ANTIGEN L6 |
| AA488029 | H. sapiens mRNA for 17-beta-hydroxysteroid dehydrogenase |
| AA488626 | Human ubiquitin-homology domain protein PIC1 mRNA, complete cds |
| AA490047 | Human alpha-CP1 mRNA, complete cds |
| AA490124 | ESTs |
| AA504554 | Human cytoskeleton associated protein (CG22) mRNA, complete cds |
| AA521243 | PUTATIVE 60S RIBOSOMAL PROTEIN |
| AA598400 | PRE-MRNA SPLICING FACTOR SRP20 |
| AA599092 | Protein phosphatase 2 (formerly 2A), catalytic subunit, alpha isoform |
| H06113 | MITOCHONDRIAL 605 RIBOSOMAL PROTEIN L3 |
| H07880 | Human chaperonin protein (Tcp20) gene complete cds |
| H70554 | ESTs |
| N53169 | Apolipoprotein C-III |
| N70794 | Acyl-Coenzyme A dehydrogenase C-4 to C-12 straight chain |
| N77514 | ESTs, Weakly similar to C16C10.10 [C. elegans] |
| N91990 | Homo sapiens peroxisomal phytanoyl-CoA aipha-hydroxylase (PAHX) mRNA, complete cds |
| R32756 | Ewing sarcoma breakpoint region 1 |
| R68102 | ESTs |
| R93124 | Dihydrodiol dehydrogenase |
| T59286 | 5-ADENOSYLMETHIONINE SYNTHETASE GAMMA FORM |
| T70122 | Ribonuclease L (2′,5′-oligoisoadenylate synthetase-dependent) inhibitor |
| T94626 | FIBRINOGEN GAMMA-A CHAIN PRECURSOR |
| W02101 | Heterogeneous nuclear ribonucleoprotein A2/B1 |
| W05553 | ESTs, Weakly similar to D9481.16 gene product [S. cerevisiae] |
| W32403 | ESTs, Moderately similar to MSG1-related protein [H. sapiens] |
| W32907 | ESTs, Weakly similar to T1208.b [C. elegans] |
| AA004759 | Homo sapiens dolichol monophosphate mannose synthase (DPM1) mRNA, partial cds |
| AA024656 | Human mRNA for KIAA0384 gene, complete cds |
| AA025195 | ESTs, Highly similar to HISTONE H2A.1 [Xenopus laevis] |
| AA063521 | Homo sapiens E1B 19K/Bcl-2-binding protein Nip3 mRNA, nuclear |
| | gene encoding mitochondrial protein, complete cds |
| AA070226 | H. sapiens mRNA for selenoprotein P |
| M193254 | Eukaryotic translation initiation factor 4E |
| AA250730 | HEAT SHOCK FACTOR PROTEIN 2 |
| AA405769 | Phosphoenolpyruvate carboxykinase 1 (soluble) |
| AA418918 | Human nuclear autoantigen GS2NA mRNA, complete cds |
| AA446682 | Homo sapiens autoantigen mRNA, complete cds |
| AA446839 | — |
| AA449834 | Human GAP SH3 binding protein mRNA, complete cds |
| AA458646 | H. sapiens mRNA for RNA polymerase II subunit |
| AA459213 | ESTs |
| AA459941 | Human PEG3 mRNA, partial cds |
| AA464346 | Human mRNA for platelet activating factor acetylhydrolase IB gamma-subunit, complete cds |
| AA480835 | Human myelodysplasia/myeloid leukemia factor 2 (MLF2) mRNA, complete cds |
| AA485911 | ER LUMEN PROTEIN RETAINING RECEPTOR 2 |
| AA486430 | Human JTV-1 (JTV-1) mRNA, complete cds |
| AA486669 | Glutathione S-transferase M1 |
| AA496780 | H. sapiens mRNA for RAB7 protein |
| AA504461 | LOW-DENSITY LIPOPROTEIN RECEPTOR PRECURSOR |
| AA598840 | Human polyhomeotic 2 homolog (HPH2) mRNA, complete cds |
| AA599078 | Signal recognition particle 54 kD protein |
| H11792 | Human putative splice factor transformer2-beta mRNA, complete cds |
| H15215 | STERYL-SULFATASE PRECURSOR |
| H29484 | Sjogren syndrome antigen B (autoantigen La) |
| H37989 | TUBULIN BETA-1 CHAIN |
| H43317 | ESTs, Weakly similar to 2-19 PROTEIN PRECURSOR [H. sapiens] |
| H51765 | ESTs, Highly similar to IG ALPHA-2 CHAIN C REGION [H.sapiens] |
| H79007 | EST |
| H94469 | ESTs, Weakly similar to T01G9.4 [C. elegans] |
| N73130 | Human clone 23722 mRNA sequence |
| N73252 | Human mRNA for proteasome subunit HsC7-I, complete cds |
| N77326 | ESTs, Highly similar to 3-HYDROXYISOBUTYRATE DEHYDROGENASE |
| | PRECURSOR [Rattus norvegicus] |
| N80741 | Homo sapiens mRNA for ATP binding protein, complete cds |
| R06417 | Junction plakoglobin |
| R09980 | ESTs, Weakly similar to !!!! ALU CLASS B WARNING ENTRY !!!! [H. sapiens] |
| R11526 | Parathymosin |
| R12473 | Adenosine kinase |
| R39430 | ESTs, Highly similar to TIF1 protein [M. musculus] |
| R41928 | Human mercurial-insensitive water channel mRNA, form 2, complete cds |
| R69307 | ESTs, Highly similar to CYTOSOL AMINOPEPTIDASE [Bos taurus] |
| T57959 | Zinc finger protein 3 (A8-51) |
| W92963 | ESTs, Highly similar to LEYDIG CELL TUMOR 10 KD PROTEIN [Rattus norvegicus] |
| AA232856 | DNA topoisomerase I |
| AA453105 | Human histone 2A-like protein (H2A/I) mRNA, complete cds |
| AA598492 | Ubiquitin-conjugating enzyme E2B (RAD6 homolog) |
| H05919 | Human mRNA for eukaryotic initiation factor 4AII |
| H92821 | Homo sapiens TTF-I interacting peptide 21 mRNA, partial cds |
| R58991 | Spermidine/spermine N1-acetyltransferase mRNA, complete cds |
| R60160 | Human topoisomerase I mRNA, complete cds |
| AA464600 | V-myc avian myelocytomatosis viral oncogene homolog |
| H54020 | Homo sapiens 9G8 splicing factor mRNA, complete cds |
| R69163 | ESTs |
| W87741 | — |
| AA017199 | Human E2 ubiquitin conjugating enzyme UbcH5C (UBCH5C) mRNA, complete cds |
| AA019459 | Human protein tyrosine kinase mRNA, complete cds |
| AA232979 | Human clone A9A2BR11 (CAC)n/(GTG)n repeat-containing mRNA |
| AA453850 | Homo sapiens FLICE-like inhibitory protein long form mRNA, complete cds |
| AA480815 | H. sapiens PRG1 gene |
| AA486728 | Vinculin |
| AA490696 | Human mRNA for protein phosphatase 2A (beta-type) |
| AA504327 | Human protein-tyrosine phosphatase (HU-PP-1) mRNA, partial sequence |
| AA598483 | Human tax1-binding protein TXBP151 mRNA, complete cds |
| H08749 | DUAL SPECIFICITY MITOGEN-ACTIVATED PROTEIN KINASE KINASE 3 |
| H78483 | Human huntingtin interacting protein (HIP2) mRNA, complete cds |
| N31467 | Human cell surface protein HCAR mRNA, complete cds |
| R05309 | ESTs, Highly similar to HYPOTHETICAL 39.5 KD PROTEIN |
| | C12G12.06C IN CHROMOSOME I [Schizosaccharomyces pombe] |
| R27552 | ESTs |
| R91904 | ESTs, Highly similar to AQUAPORIN 3 [Rattus norvegicus] |
| T94293 | Human calcium-dependent group X phospholipase A2 mRNA, complete cds |
| W03672 | ESTs |
| W96268 | Glutamate-cysteine ligase (gamma-glutamylcysteine synthetase), regulatory (308 kD) |
| AA186901 | H.sapiens mRNA for phosphoenolpyruvate carboxykinase |
| H96140 | Acyl-coA dehydrogenase |
| T72220 | PLASMA RETINOL-BINDING PROTEIN PRECURSOR |
| AA281667 | Protein kinase inhibitor [human, neuroblastoma cell line SH-SY-5Y, mRNA, 2147 nt] |
| AA411107 | Human mRNA for U1 small nuclear RNP-specific C protein |
| AA448396 | Heat shock 10 kD protein 1 (chaperonin 10) |
| AA453849 | ATP synthase, H+ transporting, mitochondrial F0 complex, subunit b, isoform 1 |
| AA456400 | Adenylosuccinate lyase |
| AA456474 | Apolipoprotein C-II |
| AA458965 | NATURAL KILLER CELLS PROTEIN 4 PRECURSOR |
| AA486514 | Prostatic binding protein |
| AA489602 | Human tumor necrosis factor type I receptor associated protein (TRAPi) mRNA, partial cds |
| AA620580 | Human mRNA for proteasome subunit HsC10-11, complete cds |
| H61449 | CARBOXYPEPTIDASE N 83 KD CHAIN |
| H68845 | H. sapiens thiol-specific antioxidant protein mRNA |
| N49629 | H. sapiens mRNA for diubiquitin |
| R28294 | GLYCINE CLEAVAGE SYSTEM H PROTEIN PRECURSOR |
| R71913 | Proteasome component C3 |
| R92281 | Cytochrome b-5 |
| T47454 | TISSUE FACTOR PATHWAY INHIBITOR PRECURSOR |
| T65907 | Famesyl diphosphate synthase (famesyl pyrophosphate |
| | synthetase, dimethylallyltranstransferase geranyltranstransferase |
| W68220 | Human mRNA for KIAA0101 gene, complete cds |
| AA112660 | Guanine nucleotide binding protein (G protein), alpha stimulating activity polypeptide 1 |
| AA167823 | Human CD27BP (Siva) mRNA, complete cds |
| AA284495 | Human mRNA for KIAA0081 gene, partial cds |
| AA287196 | Human globin gene |
| AA401111 | Glucose phosphate isomerase |
| AA443497 | Human clone 23732 mRNA, partial cds |
| AA446994 | Fibroblast growth factor receptor 4 |
| AA450265 | Proliferating cell nuclear antigen |
| AA455197 | Phospholipid hydroperoxide glutathione peroxidase |
| AA476240 | Lysyl hydroxylase |
| AA487346 | Cathepsin H |
| AA489314 | H. sapiens mRNA for gp25L2 protein |
| AA490390 | Human small acidic protein mRNA, complete cds |
| AA598582 | Ribosomal protein L27 |
| AA598863 | Human translation initiation factor eIF-3 p110 subunit gene, complete cds |
| AA599175 | Human ribosomal protein L27a mRNA, complete cds |
| AA608557 | Damage-specific DNA binding protein 1 (127 kD) |
| H06516 | Human alpha-2-macroglobulin mRNA, complete cds |
| H24954 | H. sapiens LU gene for Lutheran blood group glycoprotein |
| H50993 | ESTs, Highly similar to ALPHA-ACTININ 1, CYTOSKELETAL ISOFORM [Homo sapiens] |
| H58255 | Asialoglycoprotein receptor 1 |
| H62162 | Hepsin |
| H65395 | Human mRNA for proteasome activator hPA28 subunit beta, complete cds |
| N54494 | Prepro-plasma carboxypeptidase B |
| N59626 | Human (clone pA3) protein disulfide isomerase related protein (ERp72) mRNA, complete cds |
| N64429 | ESTs, Weakly similar to T14B4.2 gene product [C. elegans] |
| N95524 | COAGULATION FACTOR X PRECURSOR |
| R15814 | Human malate dehydrogenase (MDHA) mRNA, complete cds |
| R16957 | ESTs, Highly similar to J KAPPA-RECOMBINATION SIGNAL |
| | BINDING PROTEIN [Drosophila melanogaster] |
| R42815 | Human mRNA for KIAA0246 gene, partial cds |
| R44290 | Human cytoplasmic beta-actin gene, complete cds |
| R45183 | H. sapiens mRNA for elongations factor Tu-mitochondrial |
| R68021 | ESTs |
| T47815 | INTERFERON GAMMA UP-REGULATED I-5111 PROTEIN PRECURSOR |
| T55092 | Small nuclear ribonucleoprotein polypeptide N |
| T70109 | Succinate dehydrogenase 2, flavoprotein (Fp) subunit |
| AA031284 | Human mRNA for stac, complete cds |
| AA031398 | ESTs, Moderately similar to stac [H.sapiens] |
| AA045587 | Human TFIID subunits TAF20 and TAF15 mRNA, complete cds |
| AA055862 | Human A33 antigen precursor mRNA, complete cds |
| AA056148 | Human protein tyrosine kinase t-Ror1 (Ror1) mRNA, complete cds |
| AA115876 | H. sapiens mRNA for protease inhibitor 12 (PI12; neuroserpin) |
| AA148736 | Syndecan 4 (amphiglycan, ryudocan) |
| AA293050 | JNK ACTIVATING KINASE 1 |
| AA417654 | Fibroblast growth factor receptor 3 (achondroplasia, thanatophoric dwarfism) |
| AA418670 | Jun D proto-oncogene |
| AA428749 | PROTEIN PHOSPHATASE INHIBITOR 2 |
| AA429281 | Human DNA from overlapping chromosome 19 cosmids |
| | R31396, F25451, and R31076 containing COX6B and UPKA, genomic sequence |
| AA434504 | Human clone 23665 mRNA sequence |
| AA442092 | Catenin (cadherin-associated protein), beta 1 (88 kD) |
| AA446748 | Human mRNA for rhodanese, complete cds |
| AA452374 | Syntaxin 5A |
| AA454673 | Homo sapiens transcription factor ZFM1 isoform B3 mRNA, complete cds |
| AA455969 | Prion protein (p27-30) (Creutzfeld-Jakob disease, Gerstmann-Strausler-Scheinker |
| | syndrome, fatal familial insomnia) |
| AA456695 | Human histone H2B.1 mRNA, 3′ end |
| AA463498 | H. sapiens mRNA for alpha 4 protein |
| AA465366 | Leukotriene A4 hydrolase |
| AA480995 | NAD-dependent methylene tetrahydrofolate dehydrogenase cyclohydrolase |
| AA486313 | Low density lipoprotein-related protein-associated protein 1 |
| | (alpha-2-macroglobulin receptor-associated protein 1 |
| AA598759 | Phosphogluconate dehydrogenase |
| AA600173 | Ubiquitin-conjugating enzyme E2A (RAD6 homolog) |
| AA608514 | Human transcriptional activation factor TAFII32 mRNA, complete cds |
| AA608576 | H. sapiens mRNA for novel T-cell activation protein |
| H05899 | Human nuclear ribonucleoprotein particle (hnRNP) C protein mRNA, complete cds |
| H70498 | Human mRNA for KIAA0184 gene, partial cds |
| H72520 | RING3 PROTEIN |
| N33927 | ESTs |
| N57872 | Alanine-glyoxylate aminotransferase (oxalosis I; hyperoxaluria I; |
| | glycolicaciduria; senine-pyruvate aminotransferase) |
| N59690 | ESTs, Moderately similar to PUTATIVE SERINE/THREONINEPROTEIN |
| | KINASE PKWA [Thermomonospora curvata] |
| N66278 | — |
| N75719 | Plasminogen activator inhibitor, type I |
| N95761 | Fucosidase, alpha-L-1, tissue |
| R14760 | Human cysteine protease CPP32 isoform alpha mRNA, complete cds |
| R20770 | Human mRNA for unc-18homologue, complete cds |
| R53942 | Human mitochondrial ADP/ADT translocator mRNA, complete cds |
| R70598 | ESTs, Weakly similar to !!!! ALU SUBFAMILY J WARNING ENTRY !!!! [H.sapiens] |
| R82733 | ESTs |
| R91550 | Human arginine-rich protein (ARP) gene, complete cds |
| T54418 | H.sapiens mRNA for AEX protein |
| T60235 | Spectrin, alpha, non-erythrocytic 1 (alpha-fodrin) |
| T66816 | HISTONE H1D |
| T81972 | ESTs |
| W02116 | Human (H326) mRNA, complete cds |
| W02256 | Human (clone 8B1) Br-cadherin mRNA, complete cds |
| W53015 | ESTs, Highly similar to RAS-RELATED PROTEIN RAP-1B [Homo sapiens; Bos taurus] |
| W72621 | ESTs |
| W93510 | ESTs |
| AA047338 | PROTEASOME IOTA CHAIN |
| AA055101 | Homo sapiens NADH:ubiquinone oxidoreductase 18 kDa P subunit mRNA, nuclear gene |
| | encoding mitochondrial protein, compl cds |
| AA070997 | Proteasome (prosome, macropain) subunit, beta type, 6 |
| AA115919 | Human Bruton’s tyrosine kinase-associated protein-135 mRNA, complete cds |
| AA156940 | Homo sapiens TFAR19 mRNA, complete cds |
| AA232647 | Human mRNA for DB1, complete cds |
| AA291163 | Glutaredoxin (thioltransferase) |
| AA406535 | NADH-UBIQUINONE OXIDOREDUCTASE 75 KD SUBUNIT PRECURSOR |
| AA411640 | H. sapiens mRNA for ragA protein |
| AA418689 | DNA-DIRECTED RNA POLYMERASE II 14.4 KD POLYPEPTIDE |
| AA419108 | Annexin IV (placental anticoagulant protein II) |
| AA422058 | H. sapiens mRNA for D1075-like gene |
| AA430504 | Human cyclin-selective ubiquitin carrier protein mRNA, complete cds |
| AA443177 | Homo sapiens CaM kinase II isoform mRNA, complete cds |
| AA450227 | Human antisecretory factor-1 mRNA, complete cds |
| AA453679 | Dihydrolipoamide dehydrogenase (E3 component of pyruvate |
| | dehydrogenase complex, 2-oxo-glutarate complex, branched chai |
| | acid dehydrogenase complex) |
| AA453831 | Human mRNA for hepatoma-derived growth factor, complete cds |
| AA454947 | H. sapiens mRNA for kinase A anchor protein |
| AA455538 | NAD(P)H:menadione oxidoreductase |
| AA459292 | CDC28 protein kinase 1 |
| AA459663 | Human antioxidant enzyme AOE37-2 mRNA, complete cds |
| AA460727 | Human mRNA for clathrin coat assembly protein-like, complete cds |
| AA461065 | Thiosulfate sulfurtransferase (rhodanese) |
| AA463565 | Succinate dehydrogenase, iron sulphur (Ip) subunit |
| AA464605 | Human mRNA for KIAA0172 gene, partial cds |
| AA465386 | Human Gu protein mRNA, partial cds |
| AA480906 | Human protein kinase C-binding protein RACK7 mRNA, partial cds |
| AA486518 | Human nuclear chloride ion channel protein (NCC27) mRNA, complete cds |
| AA487651 | Heterogeneous nuclear ribonucleoprotein G |
| AA487739 | Glutamic-oxaloacetic transaminase 2, mitochondrial (aspartate aminotransferase 2) |
| AA487912 | Guanine nucleotide binding protein (G protein), beta polypeptide 1 |
| AA489261 | Human mRNA for RTP, complete cds |
| AA489400 | Human mRNA for proteasome subunit z, complete cds |
| AA490617 | Human mRNA for VRK2, complete cds |
| AA490721 | Human splicing factor SRp30c mRNA, complete cds |
| AA504348 | ESTs, Highly similar to PUTATIVE GTP-BINDING PROTEIN MOV10 [Mus musculus] |
| AA504682 | Neuroblastoma RAS viral (v-ras) oncogene homolog |
| AA521249 | Small nuclear ribonucleoprotein polypeptide B” |
| AA598637 | Human stimulator of TAR RNA binding (SRB) mRNA, complete cds |
| AA598965 | Human splicing factor SRp40-1 (SRp40) mRNA, complete cds |
| AA599116 | Small nuclear ribonucleoprotein polypeptides B and B1 |
| AA599127 | Superoxide dismutase 1 (Cu/Zn) |
| AA599177 | Cystatin C (amyloid angiopathy and cerebral hemorrhage) |
| H00817 | Homo sapiens clone 23797 and 23917 mRNA, partial cds |
| H05774 | Diacyiglycerol kinase, gamma (90 kD) |
| H15707 | H. sapiens mRNA for TRAMP protein |
| H21107 | Human mRNA for KIAA0164 gene, complete cds |
| H25917 | Human BRCA2 region, mRNA sequence CG037 |
| H47080 | Human mitochondrial ATP synthase subunit 9, P3 gene copy, mRNA, nuclear gene encoding |
| | mitochondrial protein, complete cd |
| H48420 | Prothymosin alpha |
| H70114 | ESTs |
| H71217 | ESTs |
| H93552 | ESTs |
| N52911 | — |
| N54932 | ESTs, Highly similar to HYPOTHETICAL 25.7 KD PROTEIN IN MSH1-EPT1 INTERGENIC REGION |
| | [Saccharomyces cerevisia |
| N64431 | ESTs, Highly similar to TUBULIN BETA CHAIN [Caenorhabditis elegans] |
| N69283 | Human TAR DNA-binding protein-43 mRNA, complete cds |
| N91311 | ESTs, Moderately similar to METALLOPROTEINASE INHIBITOR 1 PRECURSOR [H.sapiens] |
| R05693 | Single-stranded DNA-binding protein |
| R13434 | Crystallin zeta (quinone reductase) |
| R37286 | Human hnRNP core protein A1 |
| R43581 | Human guanine nucleotide-binding protein G-s, alpha subunit mRNA, partial cds |
| R44334 | Human 90 kD heat shock protein gene, complete cds |
| R52548 | Human superoxide dismutase (SOD-1) mRNA, complete cds |
| R54850 | H. sapiens mRNA for biphenyl hydrolase-related protein |
| R60933 | Human cytoplasmic chaperonin hTRIC5 mRNA, partial cds |
| R60946 | Prohibitin |
| R63022 | ESTs |
| R63543 | ESTs, Highly similar to OVARIAN GRANULOSA CELL 13.0 KD PROTEIN HGR74 [Homo sapiens] |
| R78607 | Homo sapiens doc-1 mRNA, complete cds |
| R93237 | ESTs |
| R94659 | ESTs |
| T40311 | Homo sapiens retinoic acid-inducible endogenous retroviral DNA |
| T53907 | COATOMER BETA’SUBUNIT |
| T64625 | Esterase D/formylglutathione hydrolase |
| T64901 | Thyroxin-binding globulin |
| T65833 | Pyruvate dehydrogenase (lipoamide) alpha 1 |
| T84762 | ESTs |
| T87077 | CDW52 antigen (CAMPATH-1 antigen) |
| T94293 | Human mRNA for KIAA0220 gene, partial cds |
| W79444 | Human mRNA for KIAA0242 gene, partial cds |
| AA581887 | EST |
| J03225 | Lipoprotein-associated coagulation inhibitor |
| X02152 | Lactate dehydrogenase A |
| AA465495 | EST, similar to Long-chain acyl-coenzyme A synthetase |
| N39662 | EST |
| AC007400 | EST |
| D90209 | Activating transcription factor 4 |
| AF014897.2 | NADH dehydrogenase subunit 2 |
| U25725 | Centromere protein F (400 kD) (CENPE kinetochore protein) |
| M23161 | Human transposon-like element mRNA |
| X04506 | Apolipoprotein B-100 |
| U84573 | procollagen-lysine 2-oxoglutarate 5-dioxygenase (lysine hydroxylase) 2 |
| AA430551 | EST |
| AJ238097.1 | Lsm5 protein |
| M34055 | pyruvate dehydrogenase El-beta subunit |
| L07594 | Transforming growth factor-beta type III receptor |
| N22016 | EST |
| AI131502 | EST, similar to ubiquitin hydrolase |
| U25725 | AH antigen |
| AB019397 | DNA topoisomerase II binding protein |
| D28118 | DB1 |
| A1307606.1 | EST, bithoraxoid-like protein |
| AA581887 | EST |
| X17644 | G1 to S phasetransition 1 (GSPT1) |
| J04977 | Ku autoimmune antigen |
| N32522 | EST, similar to Ubiquinol cytochrome C reductase core protein 2 |
| AF112219 | Esterase D/formylglutathione hydrolase |
| N26592 | EST |
| AF002697 | E1B 19K/Bd-2-binding protein Nip3 |
| AF110824.1 | PPP1R5 gene |
| AA283846 | EST |
| A1310515 | EST |
| AA805555 | EST |
| M16660 | 90-kDa heat-shock protein |
| M57230 | Interleukin 6 signal transducer (gp 130, oncostatin M receptor) |
| S72459 | cAMP-responsive enhancer binding protein, alt. spliced (CREB327) |
| X52882 | T-complex polypeptide 1 |
| M55536 | Glucose transporter pseudogene |
| AF070598 | ABC transporter |
| M86707 | Myristoyl CoA:protein N-myristoyltransferase |

SEQ ID NO: 1

What is claimed is:

1. A method of expression profiling, comprising:
(a) determining the expression levels of two or more nucleic acids in a test sample, wherein the one or more nucleic acids is selected from the group consisting of Putative cyclin G1 interacting protein, EST (W74293), Fatty-acid-coenzyme A ligase (long-chain 3), KIAA0220, KIAA0069, Acinus, Translation initiation factor eIF1(A12/SUI1), Ornithine aminotransferase (gyrate atrophy), Insulin-like growth factor binding protein 1, Metallothionein-1H, F₁F₀-ATPase synthase ƒ subunit, Ring finger protein 5, EST (H73484), XP-C repair complementing protein, Squalene epoxidase, Microsomal glutathione-S-transferase 1, Defender against cell death 1, EST (AA034268), COPII protein, KIAA0917, Corticosteroid binding globulin, Calumenin, Ubiquinol-cytochrome c reductase core protein II, SEC13 (S. cerevisiae)-like 1, EST (R51835), Human chromosome 3p21.1 gene sequence, Glutathione-S-transferase-like, Ribonuclease (RNase A family, 4), Transcription factor Dp-1, MAC30, Cyclin-dependent kinase 4, Multispanning membrane protein, Splicing factor (arginine/serine-rich 1), Cytochrome c-1, Lactate dehydrogenase-A, Pyrroline-5-carboxylate synthetase, Glutamate dehydrogenase, Pyruvate dehydrogenase (lipoamide) beta, Ribosomal protein S6 kinase (90 kD, polypeptide 3), Acetyl-coenzyme A acetyltransferase 2, Proteasome activator subunit 3 (PA28 gamma; Kᵢ), EST (N22016), EST (AI131502), Activating transcription factor 4, Transforming growth factor-beta type III receptor, EST (AA283846), EST (AI 310515) and EST (AA805555), wherein the numbers listed in parentheses is the GenBank accession number; and
(b) comparing the expression levels in the test sample with expression levels of the same nucleic acids in a control sample, wherein a difference in expression levels between the test and control samples is an indicator of a toxic response in the test sample.

2. The method of claim 1, wherein the determining step determines the expression levels of at least three nucleic acids selected from the group.

3. The method of claim 2, wherein the determining step determines the expression levels of at least five nucleic acids selected from the group.

4. The method of claim 3, wherein the determining step determines the expression levels of at least ten nucleic acids selected from the group.

5. The method of claim 1, wherein the group consists of Putative cyclin G1 interacting protein, EST (W74293), Fatty-acid-coenzyme A ligase (long-chain 3), KIAA0220, KIAA0069, Acinus, Translation initiation factor eIF1(A12/SUI1), Ornithine aminotransferase (gyrate atrophy), Insulin-like growth factor binding protein 1, Metallothionein-1H, F1F0-ATPase synthase ƒ subunit, Ring finger protein 5, EST (H73484), XP-C repair complementing protein, Squalene epoxidase, Microsomal glutathione-S-transferase 1, Defender against cell death 1, EST (AA034268), COPII protein, KIAA0917, Corticosteroid binding globulin, Calumenin, Ubiquinol-cytochrome c reductase core protein II, SEC13 (S. cerevisiae)-like 1, EST (R51835), Human chromosome 3p21.1 gene sequence, Glutathione-S-transferase-like, Ribonuclease (RNase A family, 4), Transcription factor Dp-1, MAC30, Cyclin-dependent kinase 4, Multispanning membrane protein, Splicing factor (arginine/serine-rich 1), Cytochrome c-1, Lactate dehydrogenase-A, Pyrroline-5-carboxylate synthetase, Glutamate dehydrogenase, Pyruvate dehydrogenase (lipoamide) beta, Ribosomal protein S6 kinase (90 kD, polypeptide 3), Acetyl-coenzyme A acetyltransferase 2 and Proteasome activator subunit 3 (PA28 gamma; Kᵢ).

6. The method of claim 1, wherein the group consists of lactate dehydrogenase A, activating transcription factor 4, pyruvate dehydrogenase E1-beta subunit, transforming growth factor-beta type III receptor, EST (AI131502), EST (N22016), EST (AA283846), EST (AI310515) and EST(AA805555).

7. The method of claim 1, wherein the group consists of Cytochrome c-1, F1F₀-ATPase synthase, Ubiquinol-cytochrome c reductase core protein II, Lactate dehydrogenase-A, Pyruvate dehydrogenase El-beta subunit and NADH dehydrogenase subunit 2.

8. The method of claim 1, wherein the group consists of Acinus and Defender against cell death 1.

9. The method of claim 1, wherein the group consists of XP-C repair complementing protein, Glutathione-S-transferase, Metallothionein-1H, Heat shock protein 90, cAMP-dependent transcription factor ATF-4 and EST (AI148382).

10. The method of claim 1, wherein the at least one differentially expressed nucleic acid is selected from the group consisting of Lactate dehydrogenase A, Pyruvate dehydrogenase E1-beta subunit and Transforming growth factor-beta type III receptor.

11. The method of claim 1, wherein the test sample is obtained from a test cell contacted with a potential toxicant.

12. The method of claim 11, wherein the test cell is selected from the group consisting of HepG2 cells, HL60 cells, HeLa cells and MCF7 cells.

13. The method of claim 12, wherein the test cell is a HepG2 cell.

14. The method of claim 11, wherein the test cell is a population of cells.

15. The method of claim 1, wherein the determining step is performed by differential display PCR.

16. The method of claim 1, wherein the determining step is performed utilizing a probe array.

17. The method of claim 1, wherein the determining step is performed using quantitative RT-PCR.

18. The method of claim 1, further comprising:
(c) contacting a test cell capable of expressing the two or more nucleic acids with a potential toxicant; and
(d) obtaining the test sample from the test cell;
wherein the difference in expression level(s) further indicates that the potential toxicant is an actual toxicant.

19. The method of claim 1, further comprising:
(c) contacting a test cell exposed to a known toxicant and capable of expressing the two or more nucleic acids with a potential antidote;
(d) obtaining the test sample from the test cell;
wherein the absence of the difference in expression level(s) is an indication that the potential antidote is an actual antidote.

20. An isolated nucleic acid comprising a nucleotide sequence selected from the group consisting of:
(a) a deoxyribonucleotide sequence complementary to the full-length nucleotide sequence of SEQ ID NO:1;
(b) a ribonucleotide sequence complementary to the full-length nucleotide sequence of SEQ ID NO:1; and
(c) a nucleotide sequence complementary to the deoxyribonucleotide sequence of (a) or the ribonucleotide sequence of (b).

21. An isolated nucleic acid comprising at least 20 contiguous bases from nucleotides 153 to 224 as set forth in SEQ ID NO:1 or a complementary sequence of the same length.

22. A kit for conducting toxicity analysis, comprising:
(a) at least three polynucleotide probes that hybridize under stringent conditions to different nucleic acids selected from the group consisting of Putative cyclin G1 interacting protein, EST (W74293), Fatty-acid-coenzyme A ligase (long-chain 3), KIAA0220, KIAA0069, Acinus, Translation initiation factor eIF1(A12/SUI1), Ornithine aminotransferase (gyrate atrophy), Insulin-like growth factor binding protein 1, Metallothionein-1H, F₁F₀-ATPase synthase ƒ subunit, Ring finger protein 5, EST (H73484), XP-C repair complementing protein, Squalene epoxidase, Microsomal glutathione-S-transferase 1, Defender against cell death 1, EST (AA034268), COPII protein, KIAA0917, Corticosteroid binding globulin, Calumenin, Ubiquinol-cytochrome c reductase core protein II, SEC13 (S. cerevisiae)-like 1, EST (R51835), Human chromosome 3p21.1 gene sequence, Glutathione-S-transferase-like, Ribonuclease (RNase A family, 4), Transcription factor Dp-1, MAC30, Cyclin-dependent kinase 4, Multispanning membrane protein, Splicing factor (arginine/serine-rich 1), Cytochrome c-1, Lactate dehydrogenase-A, Pyrroline-5-carboxylate synthetase, Glutamate dehydrogenase, Pyruvate dehydrogenase (lipoamide) beta, Ribosomal protein S6 kinase (90 kD, polypeptide 3), Acetyl-coenzyme A acetyltransferase 2, Proteasome activator subunit 3 (PA28 gamma; Kᵢ), EST (N22016), EST (AI131502), Activating transcription factor 4, Transforming growth factor-beta type III receptor, EST (AA283846), EST (AI 310515) and EST (AA805555); and
(b) a population of cells effective for expressing the nucleic acids to which the at least three polynucleotide probes hybridize.

23. The probes of claim 22, wherein the probes are attached to a support.

24. A kit for conducting toxicity analysis, comprising at least three different primer pairs, wherein each primer pair is effective to prime the amplification of a nucleic acid segment from different nucleic acids and each primer in the primer pairs is at least 20 nucleotides long, said different nucleic acids being selected from the group consisting of Putative cyclin G1 interacting protein, EST (W74293), Fatty-acid-coenzyme A ligase (long-chain 3), KIAA0220, KIAA0069, Acinus, Translation initiation factor eIF1(A12/SUI1), Ornithine aminotransferase (gyrate atrophy), Insulin-like growth factor binding protein 1, Metallothionein-1H, F₁F₀-ATPase synthase ƒ subunit, Ring finger protein 5, EST (H73484), XP-C repair complementing protein, Squalene epoxidase, Microsomal glutathione-S-transferase 1, Defender against cell death 1, EST (AA034268), COPII protein, KIAA0917, Corticosteroid binding globulin, Calumenin, Ubiquinol-cytochrome c reductase core protein II, SEC13 (S. cerevisiae)-like 1, EST (R51835), Human chromosome 3p21.1 gene sequence, Glutathione-S-transferase-like, Ribonuclease (RNase A family, 4), Transcription factor Dp-1, MAC30, Cyclin-dependent kinase 4, Multispanning membrane protein, Splicing factor (arginine/serine-rich 1), Cytochrome c-1, Lactate dehydrogenase-A, Pyrroline-5-carboxylate synthetase, Glutamate dehydrogenase, Pyruvate dehydrogenase (lipoamide) beta, Ribosomal protein S6 kinase (90 kD, polypeptide 3), Acetyl-coenzyme A acetyltransferase 2, Proteasome activator subunit 3 (PA28 gamma; Kᵢ), EST (N22016), EST (AI131502), Activating transcription factor 4, Transforming growth factor-beta type III receptor, EST (AA283846), EST (AI310515) and EST (AA805555); and
(b) an enzyme effective at amplifying the segments in the presence of the appropriate nucleotides.

25. A system for expression profiling, comprising:
(a) at least three reporter constructs, each reporter construct comprising a different promoter or a response element and a heterologous reporter gene operably linked to the promoter or response element, wherein the promoter or response element is from a gene selected from the group consisting of Putative cyclin G1 interacting protein, EST (W74293), Fatty-acid coenzyme A ligase (long-chain 3), KIAA0220, KIAA0069, Acinus, Translation initiation factor eIF1(A12/SUI1), Ornithine aminotransferase (gyrate atrophy), Insulin-like growth factor binding protein 1, Metallothionein-1H, F₁F₀-ATPase synthase ƒ subunit, Ring finger protein 5, EST (H73484), XP-C repair complementing protein, Squalene epoxidase, Microsomal glutathione-S-transferase 1, Defender against cell death 1, EST (AA034268), COPII protein, KIAA0917, Corticosteroid binding globulin, Calumenin, Ubiquinol-cytochrome c reductase core protein II, SEC13 (S. cerevisiae)-like 1, EST (R51835), Human chromosome 3p21.1 gene sequence, Glutathione-S-transferase-like, Ribonuclease (RNase A family, 4), Transcription factor Dp-1, MAC30, Cyclin-dependent kinase 4, Multispanning membrane protein, Splicing factor (arginine/serine-rich 1), Cytochrome c-1, Lactate dehydrogenase-A, Pyrroline-5-carboxylate synthetase, Glutamate dehydrogenase, Pyruvate dehydrogenase (lipoamide) beta, Ribosomal protein S6 kinase (90 kD, polypeptide 3), Acetyl-coenzyme A acetyltransferase 2, Proteasome activator subunit 3 (PA28 gamma; Ks), EST (N22016), EST (AI131502), Activating transcription factor 4, Transforming growth factor-beta type III receptor, EST (AA283846), EST (AI 310515) and EST (AA805555); and
(b) one or more cells that harbor the at least three reporter constructs.

26. The system of claim 25, wherein the heterologous reporter gene encodes an enzyme.

27. The system of claim 26, wherein the enzyme is selected from the group consisting of β-glucuronidase, chloramphenicol acetyltransferase, luciferase, β-galactosidase and alkaline phosphatase.

28. A method of conducting expression profiling, comprising:
(a) contacting a population of test cells with a test compound, the test cells harboring at least three reporter constructs, each reporter construct comprising a different promoter or response element and a heterologous reporter gene operably linked to the promoter or response element, wherein the promoter or response element is from a gene selected from the group consisting of Putative cyclin GI interacting protein, EST (W74293), Fatty-acid-coenzyme A ligase (long-chain 3), KIAA0220, KIAA0069, Acinus, Translation initiation factor eIF1(A12/SUI1), Ornithine aminotransferase (gyrate atrophy), Insulin-like growth factor binding protein 1, Metallothionein-1H, F₁F₀-ATPase synthase ƒ subunit, Ring finger protein 5, EST (H73484), XP-C repair complementing protein, Squalene epoxidase, Microsomal glutathione-S-transferase 1, Defender against cell death 1, EST (AA034268), COPII protein, KIAA0917, Corticosteroid binding globulin, Calumenin, Ubiquinol-cytochrome c reductase core protein II, SEC13 (S. cerevisiae)-like 1, EST (R51835), Human chromosome 3p21.1 gene sequence, Glutathione-S-transferase-like, Ribonuclease (RNase A family, 4), Transcription factor Dp-1, MAC30, Cyclin-dependent kinase 4, Multispanning membrane protein, Splicing factor (arginine/serine-rich 1), Cytochrome c-1, Lactate dehydrogenase-A, Pyrroline-5 -carboxylate synthetase, Glutamate dehydrogenase, Pyruvate dehydrogenase (lipoamide) beta, Ribosomal protein S6 kinase (90 kD, polypeptide 3), Acetyl-coenzyme A acetyltransferase 2, Proteasome activator subunit 3 (PA28 gamma; Kᵢ), EST (N22016), EST (AI131502), Activating transcription factor 4, Transforming growth factor-beta type III receptor, EST (AA283846), EST (AI310515) and EST (AA805555);
whereby if the test compound produces the toxic condition the promoters or response elements activate the transcription of the reporter gene to produce a detectable signal; and
(b) detecting the level of the detectable signal from the test cells; and
(c) comparing the level of the detectable signal in the test cells with the level of the detectable signal in a population of control cells under conditions identical to those for the test cells, except that the control cells are not contacted with the test compound, an increased level of signal in the test cells indicating that the test compound is a toxicant.


SEQUENCE LISTING (25 sequences)

SEQ ID NO: 1 - DNA, 570 bp, Artificial Sequence
  Description of Artificial Sequencenovel expressed sequence tag (EST) used as a marker for cytotoxicity
ccatatatcctgcgaagaacaaccatggcaactcggaccagcccccgcctggctgcacagaagttagcgctatccccactgagtctcggcaaagaaaatcttgcagagtcctccaaaccaacagctggtggcagcagatcacaaaaggtaaactactgtcaacatccgtctactgtttgagatccagaaaattgcagtagtacctgggtgaggattggacactgcacccccgattcaggagcgctttcaaaaagtctgaccttcttggtgtggtgtwagtcagtcagtagtgagcaagtgaccgggtgagcattacagtatcagggwacatgatctcatccttcagtcaacaggccgcttatatgtagtttgatggaaaatggcattgttacatcaaaactcagtggatttctaagaaagtttcaggcgttactgatgaaggatttgaagaggtaattttccctttcgccactggtattagtcattgtttgtttcaaactttactctcacttatctgcccccagctgctaattctttattgtttttattaatcctttactttcttaaaaa

SEQ ID NO: 2 - DNA, 22 bp, Artificial Sequence
  Description of Artificial Sequencefull length T7 22-mer 3′ primer
gtaatacgactcactatagggc

SEQ ID NO: 3 - DNA, 24 bp, Artificial Sequence
  Description of Artificial Sequencefull length M13R(-48) 24-mer 5′ universal primer
agcggataacaatttcacacagga

SEQ ID NO: 4 - DNA, 19 bp, Artificial Sequence
  Description of Artificial Sequence U-19 sequencing primer
gttttcccagtcacgacgt

SEQ ID NO: 5 - DNA, 20 bp, Artificial Sequence
  Description of Artificial SequenceR-20 sequencing primer
cagctatgaccatgattacg

SEQ ID NO: 6 - DNA, 10 bp, Artificial Sequence
  Description of Artificial Sequenceten nucleotides following M13R(-48) primer sequence in 5′ arbitrary primer (ARP) 1
cgactccaag

SEQ ID NO: 7 - DNA, 10 bp, Artificial Sequence
  Description of Artificial Sequenceten nucleotides following M13R(-48) primer sequence in 5′ arbitrary primer (ARP) 2
gctagcatgg

SEQ ID NO: 8 - DNA, 10 bp, Artificial Sequence
  Description of Artificial Sequenceten nucleotides following M13R(-48) primer sequence in 5′ arbitrary primer (ARP) 3
gaccattgca

SEQ ID NO: 9 - DNA, 10 bp, Artificial Sequence
  Description of Artificial Sequenceten nucleotides following M13R(-48) primer sequence in 5′ arbitrary primer (ARP) 4
gctagcagac

SEQ ID NO: 10 - DNA, 10 bp, Artificial Sequence
  Description of Artificial Sequenceten nucleotides following M13R(-48) primer sequence in 5′ arbitrary primer (ARP) 5
atggtcgtct

SEQ ID NO: 11 - DNA, 10 bp, Artificial Sequence
  Description of Artificial Sequenceten nucleotides following M13R(-48) primer sequence in 5′ arbitrary primer (ARP) 6
tacaacgagg

SEQ ID NO: 12 - DNA, 10 bp, Artificial Sequence
  Description of Artificial Sequenceten nucleotides following M13R(-48) primer sequence in 5′ arbitrary primer (ARP) 7
tggattggtc

SEQ ID NO: 13 - DNA, 10 bp, Artificial Sequence
  Description of Artificial Sequenceten nucleotides following M13R(-48) primer sequence in 5′ arbitrary primer (ARP) 8
tggtaaaggg

SEQ ID NO: 14 - DNA, 10 bp, Artificial Sequence
  Description of Artificial Sequenceten nucleotides following M13R(-48) primer sequence in 5′ arbitrary primer (ARP) 9
taagcctagc

SEQ ID NO: 15 - DNA, 10 bp, Artificial Sequence
  Description of Artificial Sequenceten nucleotides following M13R(-48) primer sequence in 5′ arbitrary primer (ARP) 10
gatctcagac

SEQ ID NO: 16 - DNA, 10 bp, Artificial Sequence
  Description of Artificial Sequenceten nucleotides following M13R(-48) primer sequence in 5′ arbitrary primer (ARP) 11
acgctagtgt

SEQ ID NO: 17 - DNA, 10 bp, Artificial Sequence
  Description of Artificial Sequenceten nucleotides following M13R(-48) primer sequence in 5′ arbitrary primer (ARP) 12
ggtactaagg

SEQ ID NO: 18 - DNA, 10 bp, Artificial Sequence
  Description of Artificial Sequenceten nucleotides following M13R(-48) primer sequence in 5′ arbitrary primer (ARP) 14
tccatgactc

SEQ ID NO: 19 - DNA, 10 bp, Artificial Sequence
  Description of Artificial Sequenceten nucleotides following M13R(-48) primer sequence in 5′ arbitrary primer (ARP) 17
ctgctaggta

SEQ ID NO: 20 - DNA, 10 bp, Artificial Sequence
  Description of Artificial Sequenceten nucleotides following M13R(-48) primer sequence in 5′ arbitrary primer (ARP) 18
tgatgctacc

SEQ ID NO: 21 - DNA, 10 bp, Artificial Sequence
  Description of Artificial Sequenceten nucleotides following M13R(-48) primer sequence in 5′ arbitrary primer (ARP) 19
ttttggctcc

SEQ ID NO: 22 - DNA, 10 bp, Artificial Sequence
  Description of Artificial Sequenceten nucleotides following M13R(-48) primer sequence in 5′ arbitrary primer (ARP) 20
tcgatacagg

SEQ ID NO: 23 - DNA, 16 bp, Artificial Sequence
  Description of Artificial SequenceM13R(-48) primer sequence in 5′ arbitrary primer (ARP)
acaatttcacacagga

SEQ ID NO: 24 - DNA, 17 bp, Artificial Sequence
  Description of Artificial SequenceT7 RNA polymerase sequence in anchored primer (AP)
acgactcactatagggc

SEQ ID NO: 25 - DNA, 12 bp, Artificial Sequence
  Description of Artificial SequenceT-12 in anchored primer (AP)
tttttttttttt